(19) United States
(12) Patent Application Publication
(10) Pub. No.: US 2004/0009510 A1
(43) Pub. Date: Jan. 15, 2004

(54) Allosteric nucleic acid sensor molecules

(76) Inventors: Scott Seiwert, Pacifica, CA (US); Narendra Vaish, Denver, CO (US); Shawn Zinnen, Denver, CO (US); Vasant Jadhav, Boulder, CO (US); Karl Kossen, Westminster, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/422,050

(22) Filed: Apr. 23, 2003

Related U.S. Application Data
(63) Continuation-in-part of application No. PCT/US02/35529, filed on Nov. 5, 2002.
(63) Continuation-in-part of application No. 10/286,492, filed on Nov. 1, 2002.
Application No. 10/286,492 is a continuation-in-part of application No. 10/283,858, filed on Oct. 30, 2002.
Application No. 10/283,858 is a continuation-in-part of application No. 10/056,761, filed on Jan. 23, 2002.
Application No. 10/056,761 is a continuation-in-part of application No. 09/992,160, filed on Nov. 5, 2001.
Application No. 09/992,160 is a continuation-in-part of application No. 09/877,526, filed on Jun. 8, 2001, now abandoned.
Application No. 09/877,526 is a continuation-in-part of application No. 09/800,594, filed on Mar. 6, 2001, now abandoned.
(60) Provisional application No. 60/187,128, filed on Mar. 6, 2000.

(51) Int. Cl. C12Q 1/68; C07H 21/04; C12N 9/22

(52) U.S. Cl. 435/6; 435/199; 536/24.3

(57) ABSTRACT

Nucleic acid sensor molecules and methods are provided for the detection and amplification of signaling agents using enzymatic nucleic acid constructs, including Halfzymes, multicomponent nucleic acid sensor molecules, hammerhead enzymatic nucleic acid molecules, inozymes, G-cleaver enzymatic nucleic acid molecules, zinzymes, amberzymes and DNAzymes. Also provided are kits for detection and amplification. The nucleic acid sensor molecules, methods and kits provided herein can be used in diagnostics, nucleic acid circuits, nucleic acid computers, therapeutics, target validation, target discovery, drug optimization, single nucleotide polymorphism (SNP) detection, single nucleotide polymorphism (SNP) scoring, and proteome scoring as well as other uses described herein.

This patent application claims priority as a continuation-in-part of Seiwert et al., PCT/US02/35529, filed Nov. 5, 2002 entitled “ALLOSTERIC NUCLEIC ACID SENSOR MOLECULES”, and as a continuation-in-part of Seiwert et al., U.S. Ser. No. (10/286,492), filed Nov. 1, 2002, which is a continuation-in-part of Seiwert et al., U.S. Ser. No. (10/283,858), filed Oct. 30, 2002, both entitled “DETECTION OF NUCLEIC ACIDS USING MULTICOMPONENT NUCLEIC ACID SENSOR MOLECULES”; which is a continuation-in-part of Usman et al., U.S. Ser. No. (10/056,761), filed Jan. 23, 2002, which is a continuation in part of Usman et al., U.S. Ser. No. (09/992,160), filed Nov. 5, 2001, entitled “NUCLEIC ACID SENSOR MOLECULES”, which is a continuation-in-part of Usman et al., U.S. Ser. No. (09/877,526) filed Jun. 8, 2001 which is a continuation-in-part of Usman et al. U.S. Ser. No. (09/800,594), filed Mar. 6, 2001, entitled “NUCLEIC ACID SENSOR MOLECULES”, which claims priority from Usman et al., U.S. Ser. No. (60/187,128), filed Mar. 6, 2000, entitled “A PROCESS FOR THE DETECTION OF NUCLEIC ACID USING NUCLEIC ACID CATALYSTS”. These applications are hereby incorporated by reference herein in their entirety including the drawings.

### FIELD OF THE INVENTION

This invention relates to novel molecular sensors, including multicomponent nucleic acid sensors and Halfzymes, that utilize enzymatic nucleic acid constructs whose activity can be modulated by the presence or absence of various signaling agents. The present invention further relates to the use of the enzymatic nucleic acid constructs as molecular sensors capable of modulating the activity, function, or physical properties of other molecules. The invention also relates to the use of the enzymatic nucleic acid constructs as a diagnostic application, useful in identifying signaling agents in a variety of applications, for example, in clinical, industrial, environmental, agricultural and/or research settings. The invention further relates to the use of the nucleic acid sensor constructs as a tool to identify the presence of genes and/or gene products which are indicative of a particular genotype and/or phenotype, for example a disease state, infection, or related condition within subjects. In addition, the invention relates to the use of nucleic acid sensor molecules in nucleic acid-based electronics, including nucleic acid-based circuits and computers.

### BACKGROUND OF THE INVENTION

The following is a brief description of diagnostic and sensor-based applications for nucleic acids. This summary is provided only for understanding of the invention that follows. This summary is not an admission that all of the work described below is prior art to the claimed invention.

The detection of biomolecules, for example nucleic acids, can be highly beneficial in the diagnosis of diseases or medical disorders. By determining the presence of a specific nucleic acid sequence, investigators can confirm the presence of a virus, bacterium, genetic mutation, and other conditions that can relate to a disease. Assays for nucleic acid sequences can range from simple methods for detection, such as northern blot hybridization using a radiolabeled or fluorescent probe to detect the presence of a nucleic acid molecule, to the use of polymerase chain reaction (PCR) to amplify a small quantity of a specific nucleic acid to the point at which it can be used for detection of the sequence by hybridization techniques. The polymerase chain reaction, uses DNA polymerases to logarithmically amplify the desired sequence (U.S. Pat. Nos. 4,683,195; 4,683,202) using prefabricated primers to locate specific sequences. Nucleotide probes can be labeled using dyes, fluorescent, chemiluminescent, radioactive, or enzymatic labels which are commercially available. These probes can be used to detect by hybridization, the expression of a gene or related sequences in cells or tissue samples in which the gene is a normal component, as well as to screen sera or tissue samples from humans suspected of having a disorder arising from infection with an organism, or to detect novel or altered genes as might be found in tumorigenic cells. Nucleic acid primers can also be prepared which, with reverse transcriptase or DNA polymerase and PCR, can be used for detection of nucleic acid molecules that are present in very small amounts in tissues or fluids.

PCR utilizes protein enzymes (DNA polymerase) to detect specific nucleotide sequences. PCR has several disadvantages, for example requiring a high degree of technical competence for reliability, high reagent costs, and sensitivity to contamination resulting in false positives.

Several groups to date have completed draft sequences of the entire human genome. To capitalize on this information, an effort to correlate changes in specific mRNA levels with different disease states has been initiated. The synergy of these efforts has been highly successful and there is now a wealth of information relating specific changes in gene expression to disease states. One drawback to the currently available data is that it is not always true that a disease state is reflected by changes in the level of gene expression. Increasingly, post-translational events that control the function of gene products (such as protein processing and protein phosphorylation) have been shown to play important roles in the conversion from a “well” to “diseased” phenotype. Thus, to efficiently use the data generated in the human genome project for the benefit of human health, a profile of disease-specific genomes and proteomes must be generated. Such information will be essential for the generation of treatment outcomes data that link subject and disease characteristics with future treatment events. Therefore, a clear need exists for molecular tools that can generate such disease specific genomes and proteomes or Diagnostic Molecular Profiles that correlate individual cellular and molecular events with disease outcomes profiles. These profiles can then be used to rationally drive treatment policy decisions resulting in better subject care and reductions in health care spending.

A class of enzymes which can be utilized for diagnostic and sensor purposes is enzymatic nucleic acid molecules (Kuwabara et al., 2000, Curr. Opin. Chem. Bio., 4, 669; Porta et al., 1995, Biochemistry, 13, 161; Soukup et al, 1999, TIBTECH, 17, 469; Marshall et al., 1999, Nature Struc Biol., 6, 992). The enzymatic nature of an enzymatic nucleic acid molecule can be advantageous over other sensor technologies, since the concentration of analyte necessary to generate a detectable response can be lower than that required with other sensor systems which can require amplification steps. This advantage reflects the ability of the enzymatic nucleic acid molecule to act enzymatically. Thus, a specific enzymatic nucleic acid molecule is able to amplify a given signal in response to a single recognition event. Such enzymatic nucleic acid-based sensor molecules are often referred to in the art as allosteric ribozymes or allosteric DNAzymes.

In addition, the enzymatic nucleic acid molecule is a highly specific sensor molecule that can be engineered to respond to a variety of different signaling events. The use of in vitro selection techniques can be applied to the selection of new enzymatic nucleic acid molecules that are capable of allosteric modulation. Previous work in this area has focused on combining known aptamer and enzymatic nucleic acid molecule sequences (Breaker, International PCT Publication No. WO 98/2714). Later work has revealed bridge sequences that connect the receptor and enzymatic sequence domains together. These bridging sequences function such that binding of a ligand to the receptor domain triggers a conformational change within the bridge, thus modulating phosphodiester cleavage activity of the adjoining enzymatic sequence (Breaker, International PCT Publication No. WO 00/26226).

George et al., U.S. Pat. Nos. 5,834,186 and 5,741,679, describe regulatable RNA molecules whose activity is altered in the presence of a ligand.

Shih et al., U.S. Pat. No. 5,589,332, describe a method for the use of ribozymes to detect macromolecules such as proteins and nucleic acid.

Nathan et al., U.S. Pat. No. 5,871,914, describe a method for detecting the presence of an assayed nucleic acid based on a two component ribozyme system containing a detection ensemble and an RNA amplification ensemble.

Nathan and Ellington, International PCT publication No. WO 00/24931, describe the detection of an analyte by a catalytic nucleic acid sequence which converts a nucleic acid substrate to a catalytic nucleic acid product in the presence of the analyte. The catalytic nucleic acid product is then amplified, by PCR.

Sullenger et al., International PCT publication No. WO 99/29842, describe nucleic acid mediated RNA tagging and RNA revision.

Usman et al., International PCT Publication No. WO 01/66721, describes nucleic acid sensor molecules.

Nathan et al., International PCT Publication No. WO 98/08974, describes specific cofactor-dependent ribozyme constructs.

### SUMMARY OF THE INVENTION

The present invention relates to nucleic acid-based molecular sensors whose activity can be modulated by the presence or absence of various signaling agents, ligands, and/or target signaling molecules. The invention further relates to a method for the detection of specific target signaling molecules such as nucleic acid molecules, proteins, peptides, antibodies, polysaccharides, lipids, sugars, metals, microbial or cellular metabolites, analytes, pharmaceuticals, and other organic and inorganic molecules using nucleic acid sensor molecules in a variety of analytical settings, including clinical, industrial, veterinary, genomics, environmental, and agricultural applications. The invention further relates to the use of the nucleic acid sensor molecule as molecular sensors capable of modulating the activity, function, or physical properties of other molecules. The present invention also contemplates the use of the nucleic acid sensor molecule constructs as molecular switches, capable of inducing or negating a response in a system, for example in a nucleic acid-based circuit or computer.

The invention further relates to the use of nucleic acid sensor molecules in a diagnostic application to identify the presence of a target signaling molecule such as a gene and/or gene products which are indicative of a particular genotype and/or phenotype, for example, a disease state, infection, or related condition within subjects or subject samples. The invention also relates to a method for the diagnosis of disease states or physiological abnormalities related to the expression of viral, bacterial or cellular RNA and DNA.

The present invention also relates to compounds and methods for the detection of nucleic acid molecules, polynucleotides, and/or oligonucleotides to determine the presence of infectious disease agents in a sample or subject. The invention also relates to compounds and methods for the detection of nucleic acid molecules, polynucleotides, and/or oligonucleotides in a sample or subject as markers or indicators for various diseases and/or conditions in subject. In certain embodiments, the invention relates to novel multicomponent nucleic acid sensor molecules that utilize enzymatic nucleic acid constructs whose activity can be modulated by the presence or absence of signaling agents that include nucleic acids, polynucleotides and/or oligonucleotides associated with a particular infectious agent, disease or condition. The present invention further relates to the use of the multicomponent enzymatic nucleic acid constructs as molecular sensors capable of modulating the activity, function, or physical properties of other nucleic acid molecules useful in detecting nucleic acids, polynucleotides and/or oligonucleotides associated with a particular infectious agent, disease or condition. The invention also relates to the use of the multicomponent enzymatic nucleic acid constructs as diagnostic reagents, useful in identifying such signaling agents in a variety of applications, for example, in screening biological samples or fluids for infectious disease causing agents (e.g., viruses and bacteria) or for screening biological samples or fluids for markers of various diseases or conditions in a subject (e.g., diseases or conditions having a genetic basis).

The invention further relates to the use of multicomponent nucleic acid sensor molecules in a diagnostic application to identify the presence of a target signaling molecule such as a gene and/or gene products which are indicative of a particular genotype and/or phenotype, for example, a disease state, infection, or related condition within subjects or subject samples. The invention also relates to a method for the diagnosis of disease states or physiological abnormalities related to the expression of viral, bacterial or cellular RNA and DNA.

Diagnostic applications of the nucleic acid sensor molecules include the use of the multicomponent nucleic acid sensor molecules for prospective diagnosis of disease, prognosis of therapeutic effect and/or dosing of a drug or class of drugs, prognosis and monitoring of disease outcome, monitoring of subject progress as a function of an approved drug or a drug under development, subject surveillance and screening for drug and/or drug treatment. Diagnostic applications include the use of multicomponent nucleic acid sensors for research, development and commercialization of products for the rapid detection of macromolecules, such as mammalian viral nucleic acids for the diagnosis of diseases associated with viruses, prions and viroids in humans and animals.

Diagnostic applications of the nucleic acid sensor molecules include the use of the nucleic acid sensor molecules for prospective diagnosis of disease, prognosis of therapeutic effect and/or dosing of a drug or class of drugs, prognosis and monitoring of disease outcome, monitoring of subject progress as a function of an approved drug or a drug under development, subject surveillance and screening for drug and/or drug treatment. Diagnostic applications include the use of nucleic acid sensors for research, development and commercialization of products for the rapid detection of macromolecules, such as mammalian viral nucleic acids, prions and viroids for the diagnosis of diseases associated with viruses, prions and viroids in humans and animals.

Nucleic acid sensor molecules can also be used in assays to assess the specificity, toxicity and effectiveness of various small molecules, nucleoside analogs, or non-nucleic acid drugs, or doses of a specific small molecules, nucleoside analogs or nucleic acid and non-nucleic acid drugs, against validated targets or biochemical pathways and include the use of nucleic acid sensors in assays involved in high-throughput screening, biochemical assays, including cellular assays, in vivo animal models, clinical trial management, and for mechanistic studies in human clinical studies. The nucleic acid sensor can also be used for the detection of pathogens, biochemicals, for example proteins, organic compounds, or inorganic compounds, in humans, plants, animals or samples therefrom, in connection with environmental testing or detection of biohazards. The use of the nucleic acid sensor molecules in other applications such a functional genomics, target validation and discovery, agriculture or diagnostics, for example the diagnosis of disease, or the prevention or treatment of human or animal disease is also contemplated.

In one embodiment, the system of the instant invention is an in vitro system. The in vitro system can be, for example, a sample derived from an organism, mammal, subject, plant, water, beverage, food preparation, or soil or any combination thereof. In another embodiment, the system of the instant invention is an in vivo system. The in vivo system can be, for example, a bacteria, bacterial cell, fungus, fungal cell, virus, plant, plant cell, mammal, mammalian cell, human or human cell. In another embodiment, the system can be a test sample, for example, a blood sample, serum sample, urine sample, or other tissue sample, cell extract, cell, tissue extract, or entire organism.

In one embodiment, the target signaling molecule of the instant invention is an RNA, DNA, analog of RNA or analog of DNA. In one embodiment, the target signaling molecule of the instant invention is an RNA derived from a bacteria, virus, fungi, plant or mammalian genome.

In one embodiment, the reporter molecule of the instant invention is RNA, DNA, RNA analog, or DNA analog.

In one embodiment, the reporter molecule of the instant invention comprises a detectable label selected from the group consisting of chromogenic substrate, fluorescent labels, chemiluminescent labels, and radioactive labels and enzymes. Suitable enzymes include, for example, luciferase, horseradish peroxidase, and alkaline phosphatase.

In another embodiment, the reporter molecule of the instant invention is immobilized on a solid support. Suitable solid supports include silicon-based chips, silicon-based beads, controlled pore glass, polystyrene, cross-linked polystyrene, nitrocellulose, biotin, plastics, metals and polyethylene films.

In one embodiment the sensor component of the nucleic acid sensor molecule is RNA, DNA, analog of RNA or analog of DNA.

In another embodiment, the sensor component of the nucleic acid sensor molecule is covalently attached to the nucleic acid sensor molecule by a linker. Suitable linkers include one or more nucleotides, abasic moieties, polyethers, polyamines, polyamides, peptides, carbohydrates, lipids, and polyhydrocarbon compounds, and any combination thereof.

In another embodiment, the sensor component of the nucleic acid sensor molecule is not covalently attached to the nucleic acid sensor molecule.

In one embodiment, the invention features a nucleic acid sensor molecule comprising an enzymatic nucleic acid component and one or more sensor components, wherein, in response to an interaction of a single stranded RNA (ssRNA) having a single nucleotide polymorphism (SNP) with the nucleic acid sensor molecule in a system, the enzymatic nucleic acid component catalyzes a chemical reaction resulting in a detectable response.

In another embodiment, the invention features a nucleic acid sensor molecule comprising an enzymatic nucleic acid component and one or more sensor components, wherein, in response to an interaction of a single stranded DNA (ssDNA) having a single nucleotide polymorphism (SNP) with the nucleic acid sensor molecule in a system, the enzymatic nucleic acid component catalyzes a chemical reaction resulting in a detectable response.

In yet another embodiment, the invention features a nucleic acid sensor molecule comprising an enzymatic nucleic acid component and one or more sensor components, wherein, in response to an interaction of a peptide with the nucleic acid sensor molecule in a system, the enzymatic nucleic acid component catalyzes a chemical reaction resulting in a detectable response.

In another embodiment, the invention features a nucleic acid sensor molecule comprising an enzymatic nucleic acid component and one or more sensor components, wherein, in response to an interaction of a protein with the nucleic acid sensor molecule in a system, the enzymatic nucleic acid component catalyzes a chemical reaction resulting in a detectable response.

In one embodiment, the invention features a nucleic acid sensor molecule comprising an enzymatic nucleic acid component and one or more sensor components, wherein, in response to an interaction of a single stranded RNA (ssRNA) with the nucleic acid sensor molecule in a system, the enzymatic nucleic acid component catalyzes a chemical reaction resulting in the cleavage of a predetermined nucleic acid molecule associated with a disease.

In another embodiment, the invention features a nucleic acid sensor molecule comprising an enzymatic nucleic acid component and one or more sensor components, wherein, in response to an interaction of a single stranded DNA (ssDNA) with the nucleic acid sensor molecule in a system, the enzymatic nucleic acid component catalyzes a chemical reaction resulting in the cleavage of a predetermined nucleic acid molecule associated with a disease.

In yet another embodiment, the invention features a nucleic acid sensor molecule comprising an enzymatic nucleic acid component and one or more sensor components, wherein, in response to an interaction of a peptide with the nucleic acid sensor molecule in a system, the enzymatic nucleic acid component catalyzes a chemical reaction resulting in the cleavage of a predetermined nucleic acid molecule associated with a disease.

In another embodiment, the invention features a nucleic acid sensor molecule comprising an enzymatic nucleic acid component and one or more sensor components, wherein, in response to an interaction of a protein with the nucleic acid sensor molecule in a system, the enzymatic nucleic acid component catalyzes a chemical reaction resulting in the cleavage of a predetermined nucleic acid molecule associated with a disease.

In one embodiment, the invention features a nucleic acid sensor molecule comprising an enzymatic nucleic acid component and one or more sensor components, wherein, in response to an interaction of a single stranded RNA (ssRNA) with the nucleic acid sensor molecule in a system, the enzymatic nucleic acid component catalyzes a chemical reaction resulting in ligation of a predetermined nucleic acid molecule to another predetermined nucleic acid molecule.

In another embodiment, the invention features a nucleic acid sensor molecule comprising an enzymatic nucleic acid component and one or more sensor components, wherein, in response to an interaction of a single stranded DNA (ssDNA) with the nucleic acid sensor molecule in a system, the enzymatic nucleic acid component catalyzes a chemical reaction resulting in ligation of a predetermined nucleic acid molecule to another predetermined nucleic acid molecule.

In yet another embodiment, the invention features a nucleic acid sensor molecule comprising an enzymatic nucleic acid component and one or more sensor components, wherein, in response to an interaction of a peptide with the nucleic acid sensor molecule in a system, the enzymatic nucleic acid component catalyzes a chemical reaction resulting in ligation of a predetermined nucleic acid molecule to another predetermined nucleic acid molecule.

In still another embodiment, the invention features a nucleic acid sensor molecule comprising an enzymatic nucleic acid component and one or more sensor components, wherein, in response to an interaction of a protein with the nucleic acid sensor molecule in a system, the enzymatic nucleic acid component catalyzes a chemical reaction resulting in ligation of a predetermined nucleic acid molecule to another predetermined nucleic acid molecule.

In one embodiment, the invention features a method comprising: (a) contacting a nucleic acid sensor molecule of the invention with a system comprising at least one ssRNA having a single nucleotide polymorphism (SNP) under conditions suitable for the enzymatic nucleic acid component of the nucleic acid sensor molecule to catalyze a chemical reaction resulting in a detectable response; and (b) assaying for the detectable response.

In another embodiment, the invention features a method comprising: (a) contacting a nucleic acid sensor molecule of the invention with a system comprising at least one ssDNA having a single nucleotide polymorphism (SNP) under conditions suitable for the enzymatic nucleic acid component of the nucleic acid sensor molecule to catalyze a chemical reaction resulting in a detectable response; and (b) assaying for the detectable response.

In another embodiment, the invention features a method comprising: (a) contacting a nucleic acid sensor molecule of the invention with a system comprising at least one peptide under conditions suitable for the enzymatic nucleic acid component of the nucleic acid sensor molecule to catalyze a chemical reaction resulting in a detectable response; and (b) assaying for the detectable response.

In yet another embodiment, the invention features a method comprising: (a) contacting a nucleic acid sensor molecule of the invention with a system comprising at least one protein, under conditions suitable for the enzymatic nucleic acid component of the nucleic acid sensor molecule to catalyze a chemical reaction resulting in a detectable response; and (b) assaying for the detectable response.

In one embodiment, the invention features a method comprising contacting a nucleic acid sensor molecule of the invention with a system comprising at least one ssRNA under conditions suitable for the enzymatic nucleic acid component of the nucleic acid sensor molecule to cleave a predetermined nucleic acid molecule.

In another embodiment, the invention features a method comprising the steps of contacting a nucleic acid sensor molecule of the invention with a system comprising at least one ssDNA under conditions suitable for the enzymatic nucleic acid component of the nucleic acid sensor molecule to cleave a predetermined nucleic acid molecule In yet another embodiment, the invention features a method comprising the steps of contacting a nucleic acid sensor molecule of the invention with a system comprising at least one peptide under conditions suitable for the enzymatic nucleic acid component of the nucleic acid sensor molecule to cleave a predetermined nucleic acid molecule.

In another embodiment, the invention features a method comprising the steps of contacting a nucleic acid sensor molecule of the invention with a system comprising at least one protein, under conditions suitable for the enzymatic nucleic acid component of the nucleic acid sensor molecule to cleave a predetermined nucleic acid molecule.

In one embodiment, the invention features a method comprising contacting a nucleic acid sensor molecule of the invention with a system comprising at least one ssRNA having a single nucleotide polymorphism (SNP) under conditions suitable for the enzymatic nucleic acid component of the nucleic acid sensor molecule to ligate a predetermined nucleic acid molecule to another predetermined nucleic acid molecule.

In another embodiment, the invention features a method comprising the steps of contacting a nucleic acid sensor molecule of the invention with a system comprising at least one ssDNA having a single nucleotide polymorphism (SNP) under conditions suitable for the enzymatic nucleic acid component of the nucleic acid sensor molecule to ligate a predetermined nucleic acid molecule to another predetermined nucleic acid molecule.

In yet another embodiment, the invention features a method comprising the steps of contacting a nucleic acid sensor molecule of the invention with a system comprising at least one peptide under conditions suitable for the enzymatic nucleic acid component of the nucleic acid sensor molecule to ligate a predetermined nucleic acid molecule to another predetermined nucleic acid molecule.

In another embodiment, the invention features a method comprising the steps of contacting a nucleic acid sensor molecule of the invention with a system comprising at least one protein, under conditions suitable for the enzymatic nucleic acid component of the nucleic acid sensor molecule to ligate a predetermined nucleic acid molecule to another predetermined nucleic acid molecule.

In one embodiment, the invention features a method of using the nucleic acid sensor molecules of the invention to determine the function or validate a predetermined gene target, a predetermined RNA target, a predetermined peptide target, or a predetermined protein target.

In another embodiment, the invention features a method of using the nucleic acid sensor molecules of the invention to determine a genotype or to characterize single nucleotide polymorphisms (SNPs) in a gene or genome. In another embodiment, the invention features a method of using the nucleic acid sensor molecules of the invention to determine SNP scoring.

In another embodiment, the invention features a method of using the nucleic acid sensor molecules of the invention to determine a proteome, for example a disease specific proteome or treatment specific proteome. In yet another embodiment, the invention features a method of using the nucleic acid sensor molecules of the invention to determine a proteome map or to determine proteome scoring.

In one embodiment, the invention features a method of using the nucleic acid sensor molecules of the invention to determine the dosage of a therapy used in treating a subject, to determine susceptibility of a subject to disease, to determine drug metabolism in a subject, to select a subject for a clinical trail, to determine a choice of therapy in a subject, or to treat a subject.

In another embodiment, the detection of a chemical reaction in a method of the invention is indicative of the presence of the target signaling agent in the system.

In another embodiment, the absence of a chemical reaction in a method of the invention is indicative of the system lacking the target signaling agent.

In one embodiment, a system of the invention is an in vitro system, for example, a sample derived from an organism, mammal, subject, plant, water, beverage, food preparation, or soil, or any combination thereof.

In another embodiment, a system of the invention is an in vivo system, for example, a bacteria, bacterial cell, fungus, fungal cell, virus, plant, plant cell, mammal, mammalian cell, human, or human cell. In another embodiment, the system can be a test sample, for example, a blood sample, serum sample, urine sample, or other tissue sample, cell extract, cell, tissue extract, or entire organism.

In another embodiment, a component of a nucleic acid sensor molecule of the invention comprises a hammerhead, hairpin, inozyme, G-cleaver, Zinzyme, RNase P EGS nucleic acid, DNAzyme, Amberzyme, or Class I ligase motif.

A chemical reaction of a nucleic sensor molecule of the invention can comprise, for example, cleavage of a phosphodiester internucleotide linkage, ligation of a predetermined nucleic acid molecule to the nucleic acid sensor molecule, ligation of a predetermined nucleic acid molecule to another predetermined nucleic acid molecule, isomerization, phosphorylation of a peptide or protein, dephosphorylation of a peptide or protein, RNA polymerase activity, an increase or decrease in fluorescence, an increase or decrease in enzymatic activity, an increase or decrease in the production of a precipitate, an increase or decrease in chemoluminescence, or an increase or decrease in radioactive emission.

In another embodiment, the invention features a kit comprising a nucleic acid sensor molecule of the invention.

In another embodiment, the invention features an array of nucleic acid sensor molecules comprising a predetermined number of nucleic acid sensor molecules of the invention. In one embodiment, a nucleic acid sensor molecule of the instant invention is attached to a solid surface. Preferably, the surface of the instant invention comprises silicon-based chips, silicon-based beads, controlled pore glass, polystyrene, cross-linked polystyrene, nitrocellulose, biotin, plastics, metals and polyethylene films.

In another embodiment, the invention features a nucleic acid sensor molecule comprising an enzymatic nucleic acid component and one or more sensor components, wherein, in response to an interaction of a Hepatitis C virus (HCV) peptide with the nucleic acid sensor molecule in a system, the enzymatic nucleic acid component catalyzes a chemical reaction resulting in resulting in the cleavage of a predetermined RNA molecule associated with a disease, for example Hepatitis C virus (HCV) RNA.

In yet another embodiment, the invention features a nucleic acid sensor molecule comprising an enzymatic nucleic acid component and one or more sensor components, wherein, in response to an interaction of a Hepatitis C virus (HCV) protein, for example a HCV core protein or coat protein, with the nucleic acid sensor molecule in a system, the enzymatic nucleic acid component catalyzes a chemical reaction resulting in resulting in the cleavage of a predetermined RNA molecule associated with a disease, for example HCV RNA.

In one embodiment, a nucleic acid sensor molecule of the invention comprises a sensor component having a sequence derived from the Hepatitis C virus (HCV) 5′-UTR, for example structural domains IIIa-IIIf, I, II or IV.

In another embodiment, the invention features a pharmaceutical composition comprising a nucleic acid sensor molecule in a pharmaceutically acceptable carrier.

In one embodiment, the invention features a method of administering to a cell, for example a mammalian cell or human cell, a nucleic acid sensor molecule of the invention comprising contacting the cell with the nucleic acid sensor molecule under conditions suitable for the administration. The method of administration can be in the presence of a delivery reagent, for example a lipid, cationic lipid, phospholipid, or liposome.

In another embodiment, the invention features a cell, for example a mammalian cell, such as a human cell, plant cell, bacterial cell, or fungal cell, including a nucleic acid sensor molecule of the invention.

In another embodiment, the invention features an expression vector comprising a nucleic acid sequence encoding at least one nucleic acid sensor molecule of the invention in a manner which allows expression of the nucleic acid sensor molecule.

In yet another embodiment, the invention features a mammalian cell, for example a human cell, including an expression vector of the invention.

In one embodiment, an expression vector of the invention further comprises a sequence for a nucleic acid sensor molecule complementary to an RNA having Hepatitis C virus (HCV) sequence.

In another embodiment, an expression vector of the invention comprises a nucleic acid sequence encoding two or more nucleic acid sensor molecules, which may be the same or different.

In another embodiment, a peptide contemplated by the invention is a viral peptide, for example a peptide derived from Hepatitis C virus (HCV), Hepatitis B virus (HBV), Human immunodeficiency virus (HIV), Human papilloma virus (HPV), Human T-cell lymphotroptic virus Type I (HTLV-1), Cytomegalovirus (CMV), Herpes Simplex virus (HSV), Respiratory syncytial virus (RSV), Rhinovirus, West Nile virus (WNV), Hantavirus, Ebola virus, or Encephalovirus.

In another embodiment, a protein contemplated by the invention is a viral protein, for example a protein derived from HCV, HBV, HIV, HPV, HTLV-1, CMV, HSV, RSV, Rhinovirus, WNV, Hantavirus, Ebola virus, or Encephalovirus.

In another embodiment, a predetermined RNA of the invention is associated with Hepatitis C virus (HCV) infection.

In another embodiment, the method of the instant invention is carried out more than once.

### BRIEF DESCRIPTION OF THE DRAWINGS

The file of this patent contains at least one drawing executed in color. Copies of the patent with color drawing(s) will be provided by the Patent and Trademark Office upon request and payment of the necessary fee.

FIG. 1 shows a non-limiting example of a “half-zinzyme” nucleic acid sensor molecule that is modulated by the 5′-UTR of the Hepatitis C virus (HCV 5′-UTR). The figure shows both inactive and active forms of the zinzyme sensor molecule (SEQ ID NO. 43). In the presence of the target signaling oligonucleotide (SEQ ID NO. 26) which represents the stem loop IIIB of the HCV 5′-UTR, the zinzyme sensor demonstrates an activity increase of three logs in cleaving the reporter molecule component of the sensor molecule as shown in the graph (+oligo target) as compared to the sensor molecule in the absence of the target. In the presence of the full length 350 nt. HCV 5′-UTR, the zinzyme sensor molecule demonstrates an almost one log increase in activity in cleaving the reporter molecule component of the sensor molecule.

FIGS. 2A-B shows a non-limiting example of half-zinzyme nucleic acid sensor molecule mediated detection of the HCV genome. FIG. 2A shows the structure of the 5′-UTR of the HCV genome. The sequence shown is the sequence used as an oligonucleotide target for nucleic acid sensor molecule catalysis. The purine guanosine R/G cleavage site is boxed. FIG. 2B shows the results of a half-zinzyme activity assay in which the half-zinzyme was incubated either in the presence or absence of the oligonucleotide target, or in the presence of RNase-H pre-treated HCV 5′-UTR. Half-zinzyme activity is expressed relative to the level observed in the presence of model oligonucleotide. Reactions included a 1:1 molar ratio of target to halfzyme.

FIGS. 3A-B shows a non-limiting example of nucleic acid sensor molecule activation by the protein kinase ERK2. FIG. 3A shows and assay where the ERK2 nucleic acid sensor molecule (black bars) was incubated either in the presence of ERK2, BSA, or in the absence of any protein and assayed for activity. An enzymatic nucleic acid molecule that lacks the ERK2 sensor region was similarly incubated and assayed for activity (grey bars). Activity is expressed as the rate of substrate RNA cleavage relative to the rate observed in the presence of ERK2. FIG. 3B shows a graph of ERK2 concentration dependence in with the concentration of ERK2 was varied as indicated in allozyme reactions. Activity is expressed as the rate of substrate RNA cleavage relative to the maximal rate observed.

FIGS. 4A-B shows a non-limiting example of nucleic acid sensor molecule specificity. FIG. 4A shows that the ERK2 nucleic acid sensor molecule is MAPK homolog specific. Equal amounts of the mitogen activated protein kinases ERK2, JNK, or P38 were included in reactions containing the ERK2 nucleic acid sensor molecule. Activity is expressed as the rate of substrate RNA cleavage relative to the rate observed in the presence of ERK2. FIG. 4B shows the specificity of the ERK2 nucleic acid sensor molecule for activated (phosphorylated) ERK2. An equal amount of unactivated ERK2 (solid circles) or phosphorylated (activated) ERK2 (open circles) was incubated with ERK2 nucleic acid sensor and substrate cleavage was monitored over time. A reaction performed in parallel lacked protein (squares).

FIG. 5 shows a non-limiting example of a nucleic acid sensor ligase molecule of the invention that responds to HCV RNA.

FIG. 6 shows a schematic view of the secondary structure of the HCV 5′UTR (Brown et al., 1992, Nucleic Acids Res., 20, 5041-45; Honda et al., 1999, J. Virol., 73, 1165-74). Major structural domains are indicated in bold.

FIG. 7 shows the design of a halfzyme used for SNP discrimination. The halfzyme, based on a zinzyme enzymatic nucleic acid motif, (AZB7.1, SEQ ID NO: 50) was designed in a two-part nucleic acid format where one of the parts comprises the reporter molecule covalently linked to a portion of the enzymatic nucleic acid domain of the halfzyme and the second part is provided by a sequence of HBV DNA (HBV 1887, SEQ ID NO: 51). In the presence of the HBV DNA (HBV 1887), the halfzyme assembles into an active configuration to cause cleavage of the reporter molecule. In the absence of HBV DNA (HBV 1887), the halfzyme construct is not expected to form an active conformation and therefore the reporter will not be cleaved. Six different variant sequences of HBV 1887 were tested for cleavage in the presence of the halfzyme (SNPT-2-7, SEQ ID NOS: 52-57). These variant sequences include single nucleotide substitutions at two distinct positions within the cognate DNA sequence. In addition, the corresponding RNA sequence of HBV 1887 (SEQ ID NO: 58) was tested for halfzyme cleavage.

FIG. 8 shows results from a halfzyme SNP discrimination study. In the presence of the HBV DNA sequence (HBV 1887; SEQ ID NO 51) and the corresponding RNA version of this sequence (SEQ ID NO: 58) the halfzyme attains active conformation resulting in the cleavage of the reporter sequence. Introduction of single nucleotide variations within the cognate HBV DNA sequence (SEQ ID NOS: 52-57) results in inhibition of halfzyme activity. Similarly, the halfzyme construct used herein can be designed such that the reporter is not covalently linked to a nucleic acid component of the halfzyme. Cleavage of the reporter by the halfzyme can be detected using a variety of methods, such as using FRET (fluorescent resonance energy transfer).

FIGS. 9A-D shows a non-limiting example of a nucleic acid sensor molecule activated by a protein kinase. FIG. 9A shows the design of nucleic acid sensor molecules ERK-HH and ERK-HH/M1. A pre-existing RNA ligand (sensor domain) specific for the unphosphorylated form of ERK2 was fused to a hammerhead catalytic motif through an attenuated stem II structure to produce ERK-HH. Association with substrate RNA (reporter molecule) is prevented if sequences in the 5′ substrate binding arm instead pair with sequences in stem II of the hammerhead domain (boxed). ERK-HH/M1 is identical to ERK-HH except that it contains three mutations in the ligand binding domain that prevent ERK2 association. FIG. 9B shows a graph depicting substrate cleavage over time using a protein-induced nucleic acid sensor molecule. The time course for substrate RNA cleavage promoted by ERK-HH in the presence of unphosphorylated ERK2 is shown as filled circles; the time course for substrate RNA cleavage promoted by ERK-HH in the absence of any protein is shown as open circles. Also shown is a similar analysis of ERK-HH/M1 activity in the presence or absence of unphosphorylated ERK2 (closed and open squares, respectively). The inset depicts a phosphoimage showing conversion of 5′-labeled substrate RNA (S) to product RNA (P) by ERK-HH in the presence (box) or absence (dashed box) of ERK2. FIG. 9C shows the pH independence of ERK-HH activity. Duplicate reactions containing 500 nM ERK2 were performed and k_{obs} calculated as described in Example 12. Reaction pHs were 6.5, 6.8, 7.0, 7.4, 7.7 and 8.1, and buffered with HEPES (pH<7.0) or TRIS-HCl (pH>/=7.0). Error is expressed as standard deviation. FIG. 9D shows a graph depicting substrate cleavage over time using a nucleic acid sensor molecule ERK-HH/M2. ERK-HH/M2 is identical to ERK-HH except that it contains five mutations in the stem I sequence that do not support stem I-stem II interaction. Assays were performed as described in Example 12 in the presence (filled circles) or absence (open circles) of ERK2, using ERK-HH/M2 in place of ERK-HH. The results of FIG. 9D indicate that protein-dependent nucleic acid sensor activation requires an alternate conformer. The inset shows a chematic depicting nucleic acid sensor-reporter RNA interaction in stem I of ERK-HH/M2. Stem I sequences in the sensor molecule and reporter RNA each carry five mutations that maintain sensor molecule-reporter RNA interaction, but do not support stem I-stem II interaction.

FIG. 10 is a graph showing the ERK2 concentration dependence of ERK-HH activation. ERK2 was serial diluted so that the final concentration of ERK2 in reactions varied from 500 nM to 70 pM. Activity (k_{obs}) is expressed relative to the activity observed in the absence of ERK2.

FIGS. 11A-B shows the specificity of nucleic acid sensor molecule activation. FIG. 11A shows MAPK subfamily-specific nucleic acid sensor molecule activation. ERK-HH was activated either with 500 nM of rat ERK2, Bovine serum albumin (BSA), rat JNK2 (Sigma Chemical Corp., USA), human p38α (Sigma Chemical Corp., USA), or without any protein as indicated. Activity is expressed as a percentage of the observed activity rate in the presence of 500 nM ERK2. FIG. 11B shows phosphorylation state-specific nucleic acid sensor molecule activation. FIG. 11B shows a graph depicting substrate cleavage over time using nucleic acid sensor molecule ERK-HH in the presence of unphosphorylated ERK2 (filled circles), phosphorylated ERK2 (filled squares) or in the absence of any protein (open circles). The inset shows low bis-acrylamide PAGE analysis of ppERK2 preparation. K562 cells (ATCC) were maintained at a density of 5×10⁵ cells/ml in RPMI (Gibco/Life Technologies, U.S.A.) supplemented with 10% fetal bovine serum (Gemini Bio-Products, Inc, U.S.A.) and 100 U of penicillin and streptomycin per ml. Cycling K562 cells (2×10⁷) were harvested in kinase extraction buffer, pH 7.4 (KEB: 50 mM β-glycerophosphate, 1.5 mM EGTA, 20 μg/ml aprotinin, 20 μg/ml leupeptin, 2.5 μg/ml pepstatin, 2 mM benzamidine, 1 mM DTT) and lysed with a glass Dounce homogenizer using 20 strokes with pestle A. Cell extracts were clarified by high speed centrifugation and protein concentrations were determined using the Coomassie Plus Protein Assay Reagent (Pierce, Rockford, Ill.). Unphosphorylated ERK2 is indicated by an asterisk (*).

FIGS. 12A-B shows the detection of ERK2 in mammalian cell lysates. FIG. 12A shows an SDS-PAGE of a K562 cell lysate at a final concentration of 0.5 mg/ml total protein. Cell lysates were supplemented with exogenous ERK2 at the indicated concentrations (0, 500 nm, 400 nm, 300 nm, 200 nm, 100 nm, and 50 nm). The ERK2 protein is shown as indicated by the triangle. Protein was visualized by Coomassie staining. Molecular weights of size standards in K_{D} are indicated (lane 1). FIG. 12B shows nucleic acid sensor molecule activity. ERK-HH was incubated in 20% K562 cell lysate (0.5 mg/ml protein final) with a nuclease-stabilized substrate RNA under otherwise standard reaction conditions. Cell lysates were supplemented with exogenous ERK2 at the indicated concentrations (0, 500 nm, 400 nm, 300 nm, 200 nm, 100 nm, and 50 nm). Observed activity rate is expressed relative to the observed activity rate in the presence of 500 nM ERK2 in the absence of lysate.

FIGS. 13A-B shows a solution phase assay using nucleic acid sensor molecules of the invention. FIG. 13A shows an assay schematic. Activation of ERK-HH by ERK2 promotes cleavage of a substrate RNA (reporter molecule) carrying a quenched fluorescein; the result is relief of quenching of fluorescein emission at 517 nm. A second, constitutive enzymatic nucleic acid molecule promotes cleavage of a substrate RNA (reporter molecule) carrying a quenched cyanine 3 (Cy3); the result is the relief of quenching of Cy3 emission at 568 nm. Normalized signal is derived from the ratio of fluorescein emission to Cy3 emission. FIG. 13B is a graph showing the results of a duplexed, solution-phase assay. Assays contained the indicated amounts of ERK2 and fluorophore-carrying nuclease stabilized substrate RNAs for ERK-HH and the constitutive enzymatic nucleic acid molecule. Emission at 517 nm (circles) and 568 nm (squares) was measured in the linear phase of the reaction (5.5 hours) using a Hitachi F4500 Fluorescence Spectrophotometer. Second ordinate (right) represents the normalized ERK-HH activation ratio as the ratio of fluorescein to Cy3 signals (diamonds).

FIGS. 14A-B shows a nucleic acid sensor molecule responsive to phosphorylated ERK2. FIG. 14A shows a schematic of nucleic acid sensor molecule ppERK-HH, in which a high affinity RNA ligand specific for the phosphorylated form of ERK2 was fused to the hammerhead catalytic motif using the same design elements as in FIG. 9A. FIG. 14B shows the specificity of ppERK-HH activation as indicated by the relative observed activity rate of ppERK-HH in the presence of 500 nM phosphorylated ERK2 (ppERK2), 500 nM unphosphorylated ERK2, or in the absence of any protein. Activity rate is expressed relative to k_{obs} in the presence of ppERK2.

FIG. 15 shows a non-limiting example of a generalized multicomponent nucleic acid sensor molecule construct. Multicomponent sensors or “halfzymes” are derived from constitutively active enzymatic nucleic acid molecules (left), by removing a portion of the enzymatic nucleic acid's sequence (center). A target nucleic acid completes the enzymatic nucleic acid (right). The example shown is non-limiting in that additional components (e.g. 2, 3, 4, 5 etc.) can be used to modulate the activity of the sensor construct, providing additional stringency requirements or combinations of effector molecules that can be detected by one sensor.

FIG. 16 show a non-limiting example of a Halfzyme Catalytic ‘Platform’ comprising a Class I ribozyme ligase (SEQ ID NO: 64), HCV effector nucleic acid (SEQ ID NO: 65), substrate 1 (SEQ ID NO: 66) and substrate 2 (SEQ ID NO: 67). Catalytic activity of the multicomponent sensor directs the attack of the 2′ OH of substrate 2 on the alpha-phosphate at the 5′ end of substrate 2.

FIG. 17 shows a non-limiting example of a HCV target signaling agent that can be used to modulate the activity of a sensor molecule of the invention. Stem-loop IIIB of the 5′-UTR is highly conserved. Sequence of the HCV target (or effector) is the most prominently conserved sequence in all HCV isolates.

FIG. 18 shows a non-limiting example of Directed Molecular Evolution (DME). A DNA sequence library is flanked by defined sequence for PCR. Sequence variants that are inactive (circles) are separated from functional sequences (stars) and amplified. This process is then iterated.

FIG. 19 shows a non-limiting example of a Limit of Detection (LOD) by a single turnover HCV-Halfzyme sensor. The 5′-UTR is cleaved by RNase H at sequences flanking the HCV effector when base paired to DNA oligonucleotides. The L.O.D. is shown for the 5′-UTR processed by RNase H (squares) and a synthetic oligoribonucleotide (circles).

FIG. 20 shows a non-limiting example of the DME procedure used to produce HCV-Halfzyme nucleic acid sensor molecules. The initial sequence library is produced from mixed-sequence overlapping oligonucleotides. Selection is carried out by fractionating molecules that autoligate to substrate 2 in the presence of the HCV effector based on their electrophoretic mobility. The figure inset shows the region of the HCV-Halfzyme effector sequence ‘doped’ to 30%.

FIG. 21 shows the sequence of clone 8/7 HCV-Halfzyme sensor. (see Table II for rate determinations for different clones).

FIG. 22 shows kinetic characterization of a single turnover clone 8/7 HCV-Halfzyme sensor molecule. Activity plateaus as a function of pH but not Mg2+ concentration.

FIG. 23 shows an analysis of RNA-RNA interactions. A shift in electrophoretic mobility of a labeled RNA (in this example HCV effector) by increasing concentrations of an unlabeled RNA (in this example HCV-multicomponent sensor) can be quantified and used to determine affinity.

FIG. 24 shows an example of HCV-Halfzyme sensor sequence libraries used in DME-2. Three independently produced libraries based on the clone 8/7 HCV-Halfzyme sensor contained completely random sequence.

FIG. 25 shows kinetic characterization of a HCV-Halfzyme sensor library developed through DME-2. HCV-multicomponent sensor library from DME-2 (squares) and original 8/7 HCV-multicomponent sensor (circles) were characterized for the ligation events shown above: autoligation (left) and ligation of the trinucleotide GGA to substrate 2 (right). Sub-stoichiometric amounts of substrate 2 were used to monitor a single cycle of catalysis on the right.

FIG. 26 shows a non-limiting example of Optimized HCV-Halfzymes from DME-2. Clone 38 and clone 21 HCV-Halfzymes obtained from DME-2 have similar sequence inserted into the same position in addition to the sequence changes found in 8/7 HCV-Halfzyme from DME-1.

FIG. 27 shows an example of Multiple Turnover Configuration 3. The HCV-Halfzyme directs the ligation of the same substrate 2 used in autoligation (single turnover) reactions. Substrate 1 shown is a 23 nucleotide RNA.

FIG. 28 shows non-limiting example of the optimization of conditions for HCV-Halfzyme L.O.D. determinations. Clone 21 HCV-Halfzyme signal (fraction ligated, A,C) and turnover rate (B,D) were assessed as a function of pH and substrate RNA concentration (A,B), and as a function of substrate RNA concentration and Mg2+ concentration (C,D).

FIG. 29 shows the pH dependence of catalyzed and uncatalyzed substrate RNA ligation. Clone 21 HCV-Halfzyme turnover rate in the presence (upper) and absence (lower) of HCV effector.

FIG. 30 shows the 2SD Limit of Detection of Configuration 3 constructs. HCV effector oligoribonucleotide was serially diluted so that HCV-Halfzyme reactions contained the indicated number of molecules. The horizontal bar represents background plus two standard deviations.

FIG. 31 shows an example of Multiple Turnover Configuration 1. The HCV-Halfzyme is truncated by 4 nucleotides relative to the single turnover version of the HCV-Halfzyme. Optimal substrate 2 (substrate 2-4a) forms 3 base pairs with the HCV-Halfzyme. Substrate 1 is a triphosphorylated trinucleotide.

FIG. 32 shows sequences of HCV halfzyme sequences derived from directed molecular evolution (DME) studies.

FIG. 33 shows a non-limiting example of efficient HCV sequence-activated multiple turnover Halfzymes. (A) Four sequence libraries based on the autoligation version of the clone 21 Halfzyme (black) were produced for a second iterative RNA selection. All four libraries maintained the nucleotide changes in the clone 21 Halfzyme sequence relative to the 207t Halfzyme (yellow). In one library, nearly all of the single stranded positions not representing such changes were completely randomized (blue highlight). Library complexity was sufficiently low so that all possible sequence combinations were represented in this library. Three other libraries all maintained the exact sequence of the clone 21 Halfzyme but also included an additional “domain” of random sequence at the locations indicated. The four libraries were 3′ truncated relative to the clone 21 Halfzyme so that the P7 helix was eleven base pairs in length and were used with the truncated HCV effector sequence shown (green). Substrate was extended at its 5′ end to allow for PCR. (B) The clone 21 Halfzyme isolated from iterative selection maintained all of the sequence changes produced in the clone 8/7 Halfzyme isolated from the initial iterative RNA selection (yellow) and carried an inserted region that, upon examining all members of this sequence family, had a conserved portion (blue) and a variable portion (purple). Alternate P3 helix allowed by the inserted region is shown and was tested using mutations (pink) in the 3′ side of P3 (M1) and the 5′ side of either the alternate (M1) or original (M3) P3 base pairing arrangement. Three different multiple turnover versions of the clone 21 Halfzyme were constructed by 5′ truncation (numbered blue arrows). Two guanosine residues were added to the 5′ end of each multiple turnover Halfzyme to allow efficient transcription by T7 RNA polymerase. Sequence 5′ of the arrow was independently transcribed and supplied to the appropriate Halfzyme as pppS in multiple turnover reactions. Joining regions J1/3 and J3/4 indicated (gray). (C) Kinetic analysis of autoligation of the clone 8/7 Halfzyme (red circles) or variants either carrying mutations M1 and M2 (blue squares) or mutations M1 and M3 (green triangles) in the presence of stoichiometric amounts of the HCV effector oligonucleotide. (D) Time course of multiple turnover ligation promoted by the clone 21 Halfzyme in the presence of a stochiometric amount of its effector oligonucleotide (solid red circles) or in the absence of the effector (open red circles) relative to the ligation of the substrate RNAs that is observed without Halfzyme or effector nucleic acid (blue squares).

FIG. 34 shows a non-limiting example of characterization and optimization of Halfzyme clone 21 in multiple turnover configuration 3. (A) The rate of the initial catalytic cycle of configuration 3 (blue) was compared to the rate of autoligation (red) when their respective substrate RNAs were pre-incubated as indicated. (B) Turnover rates of configuration 3 afforded by mutant S_{OH} and pppS substrate RNAs expressed relative to the initial substrate RNA pair (red and blue). P2 interaction with effector nucleic acid (black line) and potential wobble base pairs (green highlight) are indicated. Circles indicate relative rates of mutations further characterized. All substrate RNAs tested at 10 uM. (C) Turnover rate of configuration 3 afforded by original (red circles), C8U/flip-13 (blue squares) or C8U/flip-13/A5G (green diamonds) substrate RNA pairs as a function of substrate RNA concentration. (D) Turnover rate of configuration 3 as a function of MgCl₂ and KCl concentrations. (E) Maximum turnover rate of configuration 3 using the C8U/flip-13/A5G substrate RNA pair as a function of pH inferred from Lineweaver-Burk analysis of turnover rate as a function of substrate RNA concentration (closed red circles) compared to direct measurement of rate in the absence of effector nucleic acid at identical pH values (open red circles).

FIG. 35 shows a non-limiting example of Limit of Detection of a HCV sequence specific Halfzyme. (A) Calculated LOD was determined by solving equation 5 (see Example 14 herein) after substrate RNA concentration was converted to number of molecules in 5 uL reactions. Here, k_{cat} and kₘₐₓ were not corrected for the fraction of active effector-Halfzyme complex. Calculated LOD is expressed as a function of substrate RNA concentration and pH. Condition used to experimentally determine low is shown (blue circle). (B) Ligation product from duplicate reactions examining product formation as a function of HCV effector copy number. Minor species migrating more rapidly than the major species observed in some lanes is derived from N-1 pppS generated from in vitro transcription was not used for quantification. (C) Quantification of product formation as a function of HCV effector copy number from two Halfzyme reaction each from two independent serial dilutions (total 4 reactions). Dashed red line indicates LOD extrapolated from a power function fit to signal from 10⁷ to 10⁴ HCV copies to signal observed in the absence of HCV effector. Standard deviation from four separate trials amounted to less than 10% of the average Halfzyme activity in the presence of 10⁷ to 10⁴ molecules.

### DETAILED DESCRIPTION OF THE INVENTION

The present invention features compounds, compositions, methods, and kits for the detection of specific nucleic acid based target signaling agents indicative of disease causing agents or markers or disease. The target signaling agents comprise nucleic acids, polynucleotides, and/or oligonucleotides. The target signaling agents further comprise effector molecules that modulate the activity of multicomponent nucleic acid sensor molecules by providing a component to the sensor construct that can modulate the activity of the sensor molecule.

In one embodiment, the invention features a multicomponent nucleic acid sensor molecule comprising one or more enzymatic nucleic acid components, wherein, in response to an interaction of one or more effector components with an enzymatic nucleic acid sensor component in a system, the multicomponent nucleic acid sensor molecule catalyzes a chemical reaction involving ligation. In another embodiment, the ligation reaction involves covalent attachment of one reporter molecule (a first substrate) to another reporter molecule (a second substrate). In another embodiment, the ligation reaction results in the formation of a phosphodiester bond. In another embodiment, a first or second substrate comprises a terminal phosphate group. In yet another embodiment, the reporter molecule of the invention comprises one or more polynucleotides.

In another embodiment, the invention features a multicomponent nucleic acid sensor molecule comprising one or more enzymatic nucleic acid components, wherein, in response to an interaction of one or more effector components with an enzymatic nucleic acid sensor component in a system, the multicomponent nucleic acid sensor molecule catalyzes a chemical reaction involving cleavage. In another embodiment, the cleavage reaction involves phosphodiester cleavage. In yet another embodiment, the reporter molecule of the invention comprises one or more polynucleotides.

In one embodiment, the invention features a nucleic acid sensor molecule comprising an enzymatic nucleic acid component and a separate effector component, wherein the enzymatic nucleic acid is assembled from two or more separate nucleic acid molecules, wherein the separate effector component is one of the two ore more separate nucleic acid molecules that make up the enzymatic nucleic acid component of the nucleic acid sensor molecule, such the in the presence of the separate effector component, the enzymatic nucleic acid component assembles in a form necessary to enable the nucleic acid sensor molecule to catalyze a chemical reaction involving one or more reporter molecules, and wherein the effector and the reporter molecules are separate molecules.

In one embodiment, the chemical reaction catalyzed by a nucleic acid sensor molecule of the invention is a ligation reaction. In another embodiment, the ligation reaction involves covalent attachment of a first reporter molecule to a second reporter molecule. In another embodiment, the ligation reaction results in the formation of a phosphodiester bond. In yet another embodiment, the first or second reporter molecule independently comprises a terminal phosphate group.

In one embodiment, the chemical reaction catalyzed by a nucleic acid sensor molecule of the invention is a phosphodiester cleavage reaction.

In another embodiment, a reporter molecule of the invention comprises one or more polynucleotides.

In one embodiment, the enzymatic nucleic acid component of a nucleic acid sensor molecule of the invention is assembled from two separate nucleic acid molecules. In another embodiment, the enzymatic nucleic acid component of a nucleic acid sensor molecule of the invention is assembled from three separate nucleic acid molecules.

In one embodiment, the invention features a method, comprising: (a) contacting one or more enzymatic nucleic acid components of a multicomponent nucleic acid sensor molecule with a system under conditions suitable for one or more effector components that may be present in the system to interact with a enzymatic nucleic acid component of the multicomponent nucleic acid sensor molecule and to catalyze a chemical reaction involving the ligation of at least a portion of a first reporter molecule to at least a portion of a second reporter molecule; and (b) assaying for the ligation of at least a portion of a first reporter molecule to at least a portion of a second reporter molecule.

In one embodiment, the invention features a method, comprising: (a) contacting a nucleic acid sensor molecule of the invention with a system under conditions suitable for the nucleic acid sensor molecule and to catalyze a chemical reaction on a reporter molecule; and (b) assaying for the chemical reaction on the reporter molecule In another embodiment, the invention features a method, comprising: (a) contacting one or more enzymatic nucleic acid components of a multicomponent nucleic acid sensor molecule with a system under conditions suitable for one or more effector components that may be present in the system to interact with a enzymatic nucleic acid component of the multicomponent nucleic acid sensor molecule and to catalyze a chemical reaction involving phosphodiester cleavage of a reporter molecule; and (b) assaying for the cleavage reaction.

In one embodiment, a method of the invention further features treating the system under conditions for an effector component of a multicomponent nucleic acid sensor molecule is available to interact with an enzymatic nucleic acid component of a multicomponent nucleic acid sensor molecule of the invention. Such treatment can comprise the use of reagents that cleave RNA or DNA at predetermined sites or alternately cleave RNA or DNA randomly.

In one embodiment, the detection of a ligation reaction catalyzed by a nucleic acid sensor molecule of the instant invention is indicative of the presence of the effector component or target nucleic acid molecule in a system.

In another embodiment, the absence of a ligation reaction catalyzed by a nucleic acid sensor molecule of the instant invention is indicative of a system lacking the effector component or target nucleic acid molecule.

In one embodiment, the detection of a cleavage reaction catalyzed by a nucleic acid sensor molecule of the instant invention is indicative of the presence of the effector component or target nucleic acid molecule in a system.

In another embodiment, the absence of a cleavage reaction catalyzed by a nucleic acid sensor molecule of the instant invention is indicative of a system lacking the effector component or target nucleic acid molecule.

In one embodiment, the system of the instant invention is an in vitro system. The in vitro system can be, for example, a sample derived from an organism, mammal, subject, plant, water, beverage, food preparation, or soil or any combination thereof. In another embodiment, the system of the instant invention is an in vivo system. The in vivo system can be, for example, a bacteria, bacterial cell, fungus, fungal cell, virus, plant, plant cell, mammal, mammalian cell, human or human cell. In another embodiment, the system can be a test sample, for example, a blood sample, serum sample, saliva sample, urine sample, or other tissue sample, cell extract, cell, tissue extract, or entire organism.

In one embodiment, the effector component of a multicomponent nucleic acid sensor molecule of the instant invention is an RNA, DNA, analog of RNA or analog of DNA. In one embodiment, the effector component of a multicomponent nucleic acid sensor molecule of the instant invention is an RNA or DNA derived from a bacteria, virus, fungi, plant or mammalian genome. In yet another embodiment, the effector component of a multicomponent nucleic acid sensor molecule of the instant invention is a component of a system, sample, or subject.

In one embodiment, a reporter molecule of the instant invention is RNA, DNA, RNA analog, or DNA analog.

In one embodiment, the reporter molecule of the instant invention comprises a detectable label selected from the group consisting of chromogenic substrate, fluorescent labels, chemiluminescent labels, and radioactive labels and enzymes. Suitable enzymes include, for example, luciferase, horseradish peroxidase, and alkaline phosphatase.

In another embodiment, the reporter molecule of the instant invention is immobilized on a solid support. Suitable solid supports include silicon-based chips, silicon-based beads, controlled pore glass, polystyrene, cross-linked polystyrene, nitrocellulose, biotin, plastics, metals and polyethylene films.

In one embodiment an enzymatic nucleic acid component of the nucleic acid sensor molecule is RNA, DNA, analog of RNA or analog of DNA.

In another embodiment, a reporter molecule of the invention is covalently attached by a linker to one or more components of a multicomponent nucleic acid sensor molecule of the invention. Suitable linkers include one or more nucleotides, abasic moieties, polyethers, polyamines, polyamides, peptides, carbohydrates, lipids, and polyhydrocarbon compounds, and any combination thereof.

In another embodiment, a reporter molecule of the invention is not covalently attached a component of a nucleic acid sensor molecule.

In another embodiment, the invention features a kit comprising a nucleic acid sensor molecule of the invention. The kit of the invention can further include any additional reagents, reporter molecules, buffers, excipients, containers and/or devices as required described herein or known in the art, to practice a method of the invention.

In another embodiment, the invention features an array of one or more enzymatic nucleic acid components of a multicomponent nucleic acid sensor molecule of the invention comprising a predetermined number of enzymatic nucleic acid components. In one embodiment, an enzymatic nucleic acid component of the instant invention is attached to a solid surface. The surface can comprise silicon-based chips, silicon-based beads, controlled pore glass, polystyrene, cross-linked polystyrene, nitrocellulose, biotin, plastics, metals and polyethylene films.

In one embodiment, an effector component of a multicomponent nucleic acid sensor molecule of the invention comprises a sequence derived from Hepatitis C virus (HCV), Hepatitis B virus (HBV), human immunodeficiency virus (HIV), human papilloma virus (HPV), poliovirus, West Nile virus (WNV), cytomegalovirus (CMV), Herpes Simplex Virus (HSV), respiratory syncytial virus (RSV), influenza virus, rhinovirus, foot and mouth disease virus, Ebola virus, dengue fever virus, feline leukemia virus (FLV), encephalovirus.

In one embodiment, an effector component of a multicomponent nucleic acid sensor molecule of the invention comprises a sequence derived from a disease causing gene, splice variant, or from a small nucleotide polymorphism (SNP). Such sequences can be indicative of cancer, metabolic disorders, and other diseases or conditions having a genetic basis.

In one embodiment, an effector component of a multicomponent nucleic acid sensor molecule of the invention comprises a sequence derived from the Hepatitis C virus (HCV) 5′-UTR, for example structural domains IIIa-IIIf, I, II or IV.

In one embodiment, an effector component of a multicomponent nucleic acid sensor molecule of the invention comprises a sequence derived from a bacterium, such as Corynebacteria, Pneumococci, Streptococci, Staphylococci, enteric bacilli, mycobacteria, spirochetes, chlamydiae. In another embodiment, an effector component of a multicomponent nucleic acid sensor molecule of the invention comprises a sequence derived from bacterial ribosomal RNA.

In another embodiment, the invention features an expression vector comprising a nucleic acid sequence encoding at least one component of a multicomponent nucleic acid sensor molecule of the invention in a manner which allows expression of the component.

In yet another embodiment, the invention features a mammalian cell, for example a human cell, including an expression vector of the invention.

In one embodiment, the invention features a multicomponent nucleic acid sensor molecule that is used to assay the presence of a nucleic acid, polynucleotide, or oligonucleotide in a system or sample, such as a biological system or sample, which is indicative of a disease or condition in a subject, or which is indicative of a disease causing agent in the biological system or sample. Non-limiting examples of disease causing agents contemplated by the invention include viral disease causing agents (such as Hepatitis C virus (HCV), Hepatitis B virus (HBV), human immunodeficiency virus (HIV), human papilloma virus (HPV), poliovirus, West Nile virus (WNV), cytomegalovirus (CMV), Herpes Simplex Virus (HSV), respiratory syncytial virus (RSV), influenza virus, rhinovirus, foot and mouth disease virus, Ebola virus, dengue fever virus, feline leukemia virus (FLV), encephalovirus and others) and bacterial disease causing agents (such as Corynebacteria, Pneumococci, Streptococci, Staphylococci, enteric bacilli, mycobacteria, spirochetes, chlamydiae, and others).

The nucleic acids, polynucleotides, and oligonucleotides to be detected are generally referred to herein as effector components of the multicomponent nucleic acid sensor molecule. An enzymatic nucleic acid component of a multicomponent nucleic acid sensor molecule of the invention can interact with nucleic acid, polynucleotide, and/or oligonucleotide effector component to perform a ligase reaction, for example between two nucleic acid substrate molecules. Alternately, an enzymatic nucleic acid component of a multicomponent nucleic acid sensor molecule of the invention can interact with nucleic acid, polynucleotide, and/or oligonucleotide effector component to perform a cleavage reaction, for example a phosphodiester cleavage reaction in a reporter molecule. Additionally, the nucleic acid sensor molecules of the invention can detect nucleic acids, polynucleotides, and/or oligonucleotides in biological fluids (e.g. blood, urine, saliva) or in fixed, treated tissue as an indication of the presence of disease or infection, and provide sensitive reagents for diagnosis of diseases and infections or in determining the presence of infection disease agents.

In one embodiment, the invention features a multicomponent nucleic acid sensor molecule having specificity for a specific compound comprising a nucleic acid, polynucleotide, and/or oligonucleotide. Such specificity is inherent in the design of the multicomponent nucleic acid sensor molecule in that the effector component of the multicomponent nucleic acid sensor molecule is derived from the target to be detected. Such specific compounds can be associated with a specific disease having a genetic or infectious basis, for example a disease resulting from a genetic splice variant, or a nucleic acid, polynucleotide, or oligonucleotide specific to a particular genotype as in the case of hereditary diseases and conditions. In another embodiment, the invention features a multicomponent nucleic acid sensor molecule having specificity for a conserved class of nucleic acid sequences associated with a particular infectious agent or disease marker. Such classes of compounds can be associated with disease in a variety of species or can comprise classes of nucleic acid molecules encoding proteins having differing amino acid sequences and/or compositions.

The invention further includes detection methods using the multicomponent nucleic acid sensor molecules of the invention. In one embodiment, the invention provides methods for the detection of nucleic acids, polynucleotides, and oligonucleotides as markers for infectious disease causing agents and diseases or conditions having a genetic basis.

In one embodiment, the invention comprises methods useful in diagnostic and pathogenesis studies of infectious disease causing agents in biological samples and/or subjects, useful for detection, surveillance, treatment, and control of infectious disease causing agents.

In one embodiment, a component of a multicomponent nucleic acid molecule of the invention is a linear nucleic acid molecule. In another embodiment, a component of a multicomponent nucleic acid molecule of the invention is a linear nucleic acid molecule that can optionally form a hairpin, loop, stem-loop, or other secondary structure. In yet another embodiment, a component of a multicomponent nucleic acid molecule of the invention is a circular nucleic acid molecule.

In another embodiment, the effector component of a multicomponent nucleic acid sensor molecule of the invention is a single stranded oligonucleotide. In another embodiment, the effector component of a multicomponent nucleic acid sensor molecule of the invention is a double-stranded oligonucleotide.

In one embodiment, a component of a multicomponent nucleic acid sensor molecule of the invention comprises an oligonucleotide having between about 20 and about 500 nucleotides. In another embodiment, a component of a multicomponent nucleic acid sensor molecule of the invention comprises an oligonucleotide having between about 40 and about 250 nucleotides. In another embodiment, a component of a multicomponent nucleic acid sensor molecule of the invention comprises an oligonucleotide having between about 50 and about 150 nucleotides.

In one embodiment, an enzymatic nucleic acid component of a multicomponent nucleic acid sensor molecule of the invention comprises an oligonucleotide having between 20 and 500 nucleotides. In another embodiment, an enzymatic nucleic acid component of a multicomponent nucleic acid sensor molecule of the invention comprises an oligonucleotide having between about 40 and about 250 nucleotides. In another embodiment, an enzymatic nucleic acid component of a multicomponent nucleic acid sensor molecule of the invention comprises an oligonucleotide having between about 50 and about 150 nucleotides.

In one embodiment, an effector component of a multicomponent nucleic acid sensor molecule of the invention comprises an oligonucleotide having length sufficient to interact with the enzymatic nucleic acid component resulting in modulation of the multicomponent nucleic acid sensor activity. In another embodiment, an effector component of a multicomponent nucleic acid sensor molecule of the invention comprises an oligonucleotide having between about 7 and about 250 nucleotides. In another embodiment, an effector component of a multicomponent nucleic acid sensor molecule of the invention comprises an oligonucleotide having between about 8 and about 150 nucleotides. In another embodiment, an effector component of a multicomponent nucleic acid sensor molecule of the invention comprises an oligonucleotide comprising a full length RNA or DNA, such as a full length RNA transcript, tRNA or fragment thereof, or a full length DNA or fragment thereof.

In one embodiment, a reporter molecule of the invention comprises a nucleic acid molecule having one or more nucleotides. In another embodiment, a reporter molecule of the invention of the invention comprises an oligonucleotide having between about 3 and about 250 nucleotides. In another embodiment, an effector component of a multicomponent nucleic acid sensor molecule of the invention comprises an oligonucleotide having between about 4 and about 150 nucleotides.

In one embodiment, an enzymatic nucleic acid component of a multicomponent nucleic acid sensor molecule of the invention comprises an oligonucleotide having any of SEQ ID NOS: 64, 68, 69, 70, 71, 72, 73, 74, or 75.

In another embodiment, an effector component of a multicomponent nucleic acid sensor molecule of the invention comprises an oligonucleotide having SEQ ID NO: 65.

In yet another embodiment, a reporter molecule of the invention comprises an oligonucleotide having of SEQ ID NOS: 66, 67, or 76-81.

In one embodiment, the detection and/or quantification of target nucleic acids, polynucleotides, and/or oligonucleotides in a method of the invention is accomplished using a variety of methods, including detecting an increase or decrease in fluorescence, an increase or decrease in enzymatic activity, an increase or decrease in the production of a precipitate, an increase or decrease in chemoluminescence, an increase or decrease in chemiluminescence, or likewise a change in UV absorbance, phosphorescence, pH, optical rotation, isomerization, polymerization, temperature, mass, capacitance, resistance, emission of radiation, or calorimetric change.

In one embodiment, detection and/or quantitation of the presence of target nucleic acids, polynucleotides, and/or oligonucleotides in the above inventive methods can be accomplished using one or more reporter molecules. The reporter molecules can be attached to the enzymatic nucleic acid component or can be free in the sample. In one embodiment, a reporter molecule of the instant invention comprises one or more nucleic acid substrate molecules having a detectable label selected from the group consisting of chromogenic substrate, fluorescent labels, chemiluminescent labels, and radioactive labels and enzymes. Suitable enzymes include, for example, luciferase, horseradish peroxidase, and alkaline phosphatase.

In another embodiment, the reporter molecule of the instant invention is immobilized on a solid support. Suitable solid supports include silicon-based chips, silicon-based beads, controlled pore glass, polystyrene, cross-linked polystyrene, nitrocellulose, biotin, plastics, metals and polyethylene films.

The present invention features compositions and methods for the detection and/or amplification of specific target signaling agents and target signaling molecules in a system using nucleic acid sensor molecules. In one embodiment, the present invention features a nucleic acid sensor molecule comprising an enzymatic nucleic acid component and one or more sensor components wherein, in response to an interaction with a target signaling agent, the enzymatic nucleic acid component catalyzes a chemical reaction in which the activity or physical properties of a reporter molecule is modulated. Preferably, the chemical reaction in which the activity or physical properties of a reporter molecule is modulated results in a detectable response.

In one embodiment, the present invention features a nucleic acid sensor molecule comprising an enzymatic nucleic acid component and one or more sensor components wherein, in response to an interaction of a target signaling agent with the nucleic acid sensor molecule, the enzymatic nucleic acid component catalyses a chemical reaction involving covalent attachment of at least a portion of a reporter molecule.

The chemical reaction in which a reporter molecule is covalently attached to the nucleic acid sensor molecule can be, for example, a ligation, transesterification, phosphorylation, carbon-carbon bond formation, amide bond formation, peptide bond formation, and disulfide bond formation.

In another embodiment, the present invention features a nucleic acid sensor molecule comprising an enzymatic nucleic acid component and one or more sensor components wherein, in response to an interaction of a target signaling molecule with the nucleic acid sensor molecule, the enzymatic nucleic acid component carries out a chemical reaction that modulates the activity or properties of the reporter molecule. The chemical reaction in which the activity of a reporter molecule is modulated can be, for example, a phosphorylation, dephosphorylation, isomerization, polymerization, amplification, helicase activity, transesterification, ligation, hydration, hydrolysis, alkylation, dealkylation, halogenation, dehalogenation, esterification, desterification, hydrogenation, dehydrogenation, saponification, desaponification, amination, deamination, acylation, deacylation, glycosylation, deglycosylation, silation, desilation, hydroboration, epoxidation, peroxidation, carboxylation, decarboxylation, substitution, elimination, oxidation, and reduction reaction, or any combination of these reactions.

In one embodiment, the invention features a nucleic acid sensor molecule comprising an enzymatic nucleic acid component and one or more sensor components wherein, in response to an interaction of a target signaling molecule with the nucleic acid sensor molecule, the enzymatic nucleic acid component can carry out a chemical reaction involving isomerization of at least a portion of a reporter molecule.

In another embodiment, the invention features a nucleic acid sensor molecule comprising an enzymatic nucleic acid component and one or more sensor components wherein, in response to an interaction of a target signaling molecule with the nucleic acid sensor molecule, the enzymatic component catalyses a chemical reaction on a non-oligonucleotide-based portion of a reporter molecule selected from the group consisting of phosphorylation and dephosphorylation reactions.

Nucleic acid sensor molecules, including halfzymes of the invention can have a detection signal, such as from a reporter molecule. Examples of reporter molecules include nucleic acid molecules comprising various tags, probes, beacons, fluorophores, chemophores, ionophores, radio-isotopes, photophores, peptides, proteins, enzymes, antibodies, nucleic acids, and enzymatic nucleic acids or a combination thereof. The reporter molecule may optionally be covalently linked to a portion of the nucleic acid sensor molecule.

In another embodiment, the reporter molecule of the instant invention can be a molecular beacon, small molecule, fluorophore, chemophore, ionophore, radio-isotope, photophore, peptide, protein, enzyme, antibodie, nucleic acid, and enzymatic nucleic acid or a combination thereof (see, for example, Singh et al., 2000, Biotech., 29, 344; Lizardi et al., U.S. Pat. Nos. 5,652,107 and 5,118,801).

Using such reporter molecules and others known in the art, the detectable response of the instant invention can be monitored by, for example, a change in fluorescence, color change, UV absorbance, phosphorescence, pH, optical rotation, isomerization, polymerization, temperature, mass, capacitance, resistance, and emission of radiation.

Detection of the target signaling event via the chemical reaction or the change in activity or physical properties of the reporter molecule can be assayed by methods known in the art. Amplification of the target signaling event via the chemical reaction or the change in activity or physical properties of the reporter molecule can be accomplished by methods known in the art, for example, modulating polymerase activity. Modulation of polymerase activity can increase polymerization in a chemical reaction, for example, a polymerase chain reaction (PCR) system, resulting in amplification of a target signaling molecule or reporter molecule.

In any of the above-described inventive methods, the system can be an in vitro system. The in vitro system can be, for example, a sample derived from an organism, mammal, subject, plant, water, beverage, food preparation, or soil, or any combination thereof. In any of the above-described inventive methods, the enzymatic nucleic acid component of said nucleic acid sensor molecule can be a hammerhead, hairpin, inozyme, G-cleaver, Zinzyme, RNase P EGS nucleic acid, Class I ligase and Amberzyme motif. Also, in any of the above-described inventive methods, the enzymatic nucleic acid component of said nucleic acid sensor molecule can be a DNAzyme.

In any of the above-described methods, the detection of a chemical reaction is indicative of the presence of the target signaling molecule in the system. In any of the above-described methods, the absence of a chemical reaction is indicative of the system lacking the target signaling molecule.

In one embodiment, the reporter molecule of the instant invention is selected from the group consisting of molecular beacons, small molecules, fluorophores, chemophores, ionophores, radio-isotopes, photophores, peptides, proteins, enzymes, antibodies, nucleic acids, and enzymatic nucleic acids or a combination thereof (see for example in Singh et al., 2000, Biotech., 29, 344; Lizardi et al., U.S. Pat. Nos. 5,652,107 and 5,118,801).

Using such reporter molecules and others known in the art, the detectable response of the instant invention can be monitored by, for example, a change in fluorescence, color change, UV absorbance, phosphorescence, pH, optical rotation, isomerization, polymerization, temperature, mass, capacitance, resistance, and emission of radiation.

Detection of the target signaling event via the chemical reaction or the change in activity or physical properties of the reporter molecule can be assayed by methods known in the art. Amplification of the target signaling event via the chemical reaction or the change in activity or physical properties of the reporter molecule is accomplished by methods known in the art, for example, modulating polymerase activity. Modulation of polymerase activity can increase polymerization in a chemical reaction, for example, a polymerase chain reaction (PCR) system, resulting in amplification of a target signaling molecule or reporter molecule.

In any of the above-described inventive methods, the system can be an in vitro system. The in vitro system can be a sample derived from, for example, an organism, mammal, subject, plant, water, beverage, food preparation, or soil, or any combination thereof.

In any of the above described methods, the target signaling molecule can be an RNA, DNA, analog of RNA or analog of DNA. Thus, for example, the reporter molecule can be an RNA, DNA, RNA analog, or DNA analog. Also, in any of the described methods, wherein the targeting signaling molecule is an RNA, preferably the RNA is derived from a bacteria (e.g. Corynebacteria, Pneumococci, Streptococci, Staphylococci, enteric bacilli, mycobacteria, spirochetes, chlamydiae), virus (e.g. Hepatitis C virus (HCV), Hepatitis B virus (HBV), human immunodeficiency virus (HIV), human papilloma virus (HPV), poliovirus, West Nile virus (WNV), Human T-cell Lymphotroptic Virus Type 1 (HTLV-1), cytomegalovirus (CMV), Herpes Simplex Virus (HSV), respiratory syncytial virus (RSV), influenza virus, rhinovirus, foot and mouth disease virus, ebola virus, dengue fever virus, feline leukemia virus (FLV)), fungi (e.g. genera Aspergillus Penicillium and Cladosporium), plant (e.g. corn, soy, cotton, wheat) or mammalian (e.g. human, mouse, rat, cat, dog, monkey) genome.

In another embodiment, the invention features a method of detecting and/or amplifying a target signaling molecule, wherein the target signaling molecule is RNA sequence derived from a virus (e.g. Hepatitis C virus (HCV), Hepatitis B virus (HBV), human immunodeficiency virus (HIV), human papilloma virus (HPV), poliovirus, West Nile virus (WNV), cytomegalovirus (CMV), Herpes Simplex Virus (HSV), respiratory syncytial virus (RSV), influenza virus, rhinovirus, foot and mouth disease virus, ebola virus, dengue fever virus, feline leukemia virus (FLV)), fungi (e.g. genera Aspergillus Penicillium and Cladosporium), plant (e.g. corn, soy, cotton, wheat) or mammalian (e.g. human, mouse, rat, cat, dog, monkey) genome, bacteria (e.g. Corynebacteria, Pneumococci, Streptococci, Staphylococci, enteric bacilli, mycobacteria, spirochetes, chlamydiae), mycoplasma or other infectious disease agent, in a system, where the system is a biological sample from a subject, animal, blood, food material, water, and/or other potential sources for infectious disease agents. The method comprises the steps of (1) contacting the system with the nucleic acid sensor molecule, where the nucleic acid sensor molecule comprises an sensor component and an enzymatic nucleic acid component, under conditions suitable for preferential interaction of the sensor component with the target signaling molecule that can be present in the system; (2) contacting the system with a reporter molecule under conditions suitable for the enzymatic nucleic acid component of the nucleic acid sensor molecule to catalyze a reaction with the reporter molecule; and (3) detecting the target signaling molecule by measuring any reaction catalyzed in (2).

In another embodiment, the invention features a method of the detecting and/or amplifying a target signaling molecule, wherein the target signaling molecule is RNA sequence derived from a virus (e.g. Hepatitis C virus (HCV), Hepatitis B virus (HBV), human immunodeficiency virus (HIV), human papilloma virus (HPV), poliovirus, West Nile virus (WNV), cytomegalovirus (CMV), Herpes Simplex Virus (HSV), respiratory syncytial virus (RSV), influenza virus, rhinovirus, foot and mouth disease virus, ebola virus, dengue fever virus, feline leukemia virus (FLV)), fungi (e.g. genera Aspergillus Penicillium and Cladosporium), plant (e.g. corn, soy, cotton, wheat) or mammalian (e.g. human, mouse, rat, cat, dog, monkey) genome, bacteria (e.g. Corynebacteria, Pneumococci, Streptococci, Staphylococci, enteric bacilli, mycobacteria, spirochetes, chlamydiae), mycoplasma or other infectious disease agent, or other potential sources for infectious disease agents. The method comprises the steps of (1) contacting a reporter molecule with a mixture comprising a system and a nucleic acid sensor molecule having an enzymatic nucleic acid component and a sensor component, under conditions suitable for the enzymatic nucleic acid component of the nucleic acid sensor molecule to interact with the reporter molecule to catalyze a reaction; and (2) detecting a target signaling molecule by measuring the reaction catalyzed in (1). If the target signaling molecule is not present in the system, then the enzymatic nucleic acid component will not catalyze a reaction with the reporter molecule and there will not be a signal to measure.

In another embodiment, one or more nucleic acid sensor molecules are attached to a solid support, for example, a silicon-based surface. Each nucleic acid sensor molecule can be attached via one of its termini by a spacer molecule to allow the nucleic acid sensor molecule to adopt the appropriate conformations without hindrance from the underlying solid support. A test mixture is contacted with one or more nucleic acid sensor molecules, and the mixture is contacted with the solid support. Measurement of a signal generated by the nucleic acid sensor molecule in response to interaction with a target signaling molecule at each address of the array reveals the concentration of each target signaling molecule in the test mixture.

In any of the above methods, the enzymatic nucleic acid component of said nucleic acid sensor molecule can be a hammerhead, hairpin, inozyme, G-cleaver, Zinzyme, RNase P, EGS nucleic acid, or Amberzyme motif.

In any of the above methods, the enzymatic nucleic acid component of said nucleic acid sensor molecule can be a DNAzyme.

In any of the above methods, the reporter molecule can comprise a detectable label selected from the group consisting of chromogenic substrate, fluorescent labels, chemiluminescent labels, and radioactive labels.

In any of the above methods, the reporter molecule can be immobilized on a solid support, preferably comprising silicon-based chips, silicon-based beads, controlled pore glass, polystyrene, cross-linked polystyrene, nitrocellulose, biotin, plastics, metals and polyethylene films.

In one embodiment of the inventive method, the sensor component of the nucleic acid sensor molecule is RNA, DNA, analog of RNA or analog of DNA.

In another embodiment, the sensor component of the nucleic acid sensor molecule is covalently attached to the nucleic acid sensor molecule by a linker. Suitable linkers include, for example, one or more nucleotides, abasic moiety, polyether, polyamine, polyamide, peptide, carbohydrate, lipid, and polyhydrocarbon compounds, and any combination thereof.

In another embodiment, the sensor component of the nucleic acid sensor molecule is not covalently attached to the nucleic acid sensor molecule.

In one embodiment, the nucleic acid sensor molecules of the invention are used to detect target signaling agents involved in human and animal disease, for example viruses, bacteria, proteins, other pathogens and toxins. Examples of viral target signaling agents include but are not limited to Hepatitis C virus (HCV), Hepatitis B virus (HBV), human immunodeficiency virus (HIV), human papilloma virus (HPV), poliovirus, West Nile virus (WNV), cytomegalovirus (CMV), Herpes Simplex Virus (HSV), respiratory syncytial virus (RSV), influenza virus, rhinovirus, foot and mouth disease virus, ebola virus, dengue fever virus, feline leukemia virus (FLV), and others. Examples of bacterial target signaling agents include but are not limited to Corynebacteria, Pneumococci, Streptococci, Staphylococci, enteric bacilli, mycobacteria, spirochetes, chlamydiae, and others.

Examples of protein target signaling agents include but are not limited to prions, for example CVJ and BSE associated prions, signal transduction proteins, tyrosine kinases, phosphatases, phosphorylases, dephosphorylases, polymerases and others. Examples of other parasite target signaling agents include but are not limited to pathogenic agents related to malaria, lyme disease (Borrelia burgdorferi), sleeping sickness, giardia, and cryptsporidia.

Examples of toxin target signaling agents include but are not limited to lead, mercury, asbestos, pesticides, herbicides, PCBs, and other organic and inorganic compounds.

The present invention also provides kits for the detection of particular targets in test mixtures. The kit comprises separate components containing solutions of a nucleic acid sensor molecule specific for a particular target signaling agent, and containing solutions of the appropriate reporter molecules. In some embodiments, the kit comprises a solid support to which is attached the nucleic acid sensor molecule to the particular target. In further embodiments, the kit further comprises a component containing a standardized solution of the target. With this solution, it is possible for the user of the kit to prepare a graph or table of the detectable signal (for example, fluorescence units vs. target concentration); this table or graph is then used to determine the concentration of the target in the test mixture. Devices that automate the manipulation of such kits, perform the repeated function of the kits, combine various steps of kits, or that generate data from the kits are further contemplated by the instant invention.

In one embodiment, the nucleic acid sensor molecules (allozymes) are used to detect the presence of or absence of single stranded RNA (ssRNA) in a system, for example in a blood sample, cell extract, cell, or entire organism. An array of nucleic acid sensor molecules, for example when attached to a surface such as a chip or bead, can be used to detect and profile ssRNA in a system. As such, nucleic acid sensor molecules can be used in the analysis and/or profiling of gene expression in vitro or in vivo. The information generated by the nucleic acid sensor array can be used in mapping gene expression patterns and genotyping for various purposes, for example in target discovery, target validation, drug discovery, determining susceptibility to disease, determining the potential effect of various treatments or therapies, predicting drug metabolism or drug response, selecting candidates for clinical trials, and for managing the treatment of disease in individual subjects.

In another embodiment, the nucleic acid sensor molecules (allozymes) are used to detect the presence of or absence of single nucleotide polymorphisms (hereinafter “SNPs”) or single stranded DNA (ssDNA) in a system, for example in a blood sample, cell extract, cell, or entire organism. An array of nucleic acid sensor molecules, for example when attached to a surface such as a chip or bead, can be used to detect and profile SNPs or ssDNA in a system. As such, nucleic acid sensor molecules can be used in SNP discovery, detection, and scoring. In a non-limiting example, a plurality of nucleic acid sensor molecules is used to screen a fetus, infant, child or adult for genetic defects based on the SNP profile of the fetus, infant, child or adult. A sample of genetic material is obtained from, for example amniotic fluid, chorionic villus, blood, or hair and is contacted with an array of nucleic acid sensor molecules. The array of nucleic acid sensor molecules comprises a SNP library such that the presence of any predetermined SNP is indicated by the corresponding nucleic acid sensor by measuring the extent of the signal produced when the nucleic acid sensor interacts with the SNP, for example by measuring fluorescence, color change, precipitate deposition, voltage or current. For example, a nucleic acid computer device of the invention can be integrated into the nucleic acid sensor array such that the output of the array is recorded electronically and can be subsequently downloaded into a database. An individual SNP profile, for example, a list of particular SNPs comprising a genotype, is established from the signals generated by the nucleic acid sensor array. As such, treatment of the fetus, infant, child or adult can be initiated before symptoms arise.

In another embodiment, the information generated by the nucleic acid sensor array can be used in genotyping for various purposes, for example in target discovery, target validation, drug discovery, determining susceptibility to disease, determining the potential effect of various treatments or therapies, predicting drug metabolism or drug response, selecting candidates for clinical trials, and for managing the treatment of disease in individual subjects.

In another embodiment, the nucleic acid sensor molecules (allozymes) are used to detect the presence of or absence peptides and/or proteins in a system, for example in a blood sample, cell extract, cell, or entire organism. These nucleic acid molecules can be used in place of Elisa or Western Blot analysis, and provide a broader array of criteria to differentiate proteins and peptides in vivo. The nucleic acid sensor molecules can be used to differentiate proteins or peptides that differ in sequence, conformation, activation state or phosphorylation state, or by other post-translational modifications. An array of nucleic acid sensor molecules, for example when attached to a surface such as a chip or bead, can be used to detect and profile peptides and/or proteins in a system. As such, nucleic acid sensor molecules can be used in proteome discovery, detection, and scoring. In a non-limiting example, a plurality of nucleic acid sensor molecule is used to screen a fetus, infant, child or adult's proteome. A sample of genetic material is obtained from, for example amniotic fluid, chorionic villus, blood, or hair and is contacted with an array of nucleic acid sensor molecules. The array of nucleic acid sensor molecules comprises a proteome library such that the presence of any predetermined peptide or protein is indicated by the corresponding nucleic acid sensor by measuring the extent of the signal produced when the nucleic acid sensor interacts with the peptide or protein, for example by measuring fluorescence, color change, precipitate deposition, voltage or current. For example, a nucleic acid computer device of the invention can be integrated into the nucleic acid sensor array such that the output of the array is recorded electronically and can be subsequently downloaded into a database. The information generated by the nucleic acid sensor array can be used in diagnostic molecular profiling applications such as protien mapping or profiling for various purposes, for example in target discovery, target validation, drug discovery, determining susceptibility to disease, determining the potential effect of various treatments or therapies, predicting drug metabolism or drug response, selecting candidates for clinical trials, and for managing the treatment of disease in individual subjects.

In one embodiment, the nucleic acid sensor molecules (allozymes) of the invention are used for in vivo applications, for example in vivo ELISA, drug screening, and gene regulation. In vivo ELISA is essentially equivalent to western blot analysis. An allozyme specific to analyte, for example DNA, RNA, protein, small molecule, metabolite etc., can be constitutively expressed along with green fluorescent protein (GFP). The allozyme is designed such that when activated it cleaves GFP mRNA thus inhibiting GFP expression. In the presence of an analyte, the GFP signal would not be observed and in the absence of the analyte, full expression of GFP would be achieved. Thus, by monitoring GFP expression the analyte concentration (e.g. protein expression) can be calculated. Similarly in vivo drug screening can be achieved using a similar system. This system would give direct IC50 and EC50 values. In one embodiment, nucleic acid sensor molecule of the invention (allozymes) can be used to modulate gene expression and the expression of RNA and protein in vivo. These nucleic acid sensor molecules are designed to respond to a signaling agent, for example, a gene, SNP, mutant protein, wild-type protein, overexpressed protein, mutant RNA, wild-type RNA, compounds, metals, polymers, other molecules and/or drugs in a system., which in turn modulates the activity of the nucleic acid sensor molecule. In response to interaction with a predetermined signaling agent, the nucleic acid sensor molecule's activity is activated or inhibited such that the expression of a particular target is selectively down-regulated. The target can comprise a wild-type protein or RNA, mutant protein or RNA, and/or a predetermined cellular component that modulates gene expression or protein activity. In a specific example, nucleic acid sensor molecules that are activated by interaction with an RNA encoding a target protein are used as therapeutic agents in vivo. The presence of RNA encoding the target protein activates the nucleic acid sensor molecule that subsequently cleaves the RNA encoding the target protein, resulting in the inhibition of protein expression. In this manner, cells that express the target protein are selectively targeted for therapeutic activity.

In another non-limiting example, an allozyme can be activated by a predetermined protein, peptide, or mutant polypeptide that causes the allozyme to inhibit the expression of the gene encoding the protein, peptide, or mutant polypeptide, by, for example, cleaving RNA encoded by the gene. In this non-limiting example, the allozyme acts as a decoy to inhibit the function of the protein, peptide, or mutant polypeptide and also inhibit the expression of the protein, peptide, or mutant polypeptide once activated by the protein, peptide, or mutant polypeptide.

Preferably, a nucleic acid molecule of the instant invention is between 13 and 500 nucleotides in length. For example, nucleic acid sensor molecules of the invention are preferably between 25 and 300 nucleotides in length, more preferably between 30 and 150 nucleotides in length, e.g., 34, 36, 38, 46, 47, 56, 65, 78, or 136 nucleotides in length. Exemplary DNAzymes of the invention are preferably between 15 and 400 nucleotides in length, more preferably between 25 and 150 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Those skilled in the art will recognize that all that is required is for the nucleic acid molecule to be of length and conformation sufficient and suitable for the nucleic acid molecule to catalyze a reaction contemplated herein. The length of the nucleic acid molecules of the instant invention are not limiting within the general limits stated.

In a preferred embodiment, the invention provides a method for producing a class of nucleic acid-based diagnostic agents that exhibit a high degree of specificity for the target signaling molecule. In additional embodiments, the invention features a method of detecting target signaling molecules or signaling agents in both in vitro and in vivo applications. In vitro diagnostic applications can comprise both solid support based and solution based chip, multichip-array, micro-well plate, and micro-bead derived applications as are commonly used in the art. In vivo diagnostic applications can include but are not limited to cell culture and animal model based applications, comprising differential gene expression arrays, FACS based assays, diagnostic imaging, and others.

By “signaling agent” or “target signaling agent” is meant a chemical or physical entity capable of interacting with a nucleic acid sensor molecule, specifically a sensor component of a nucleic acid sensor molecule, in a manner that causes the nucleic acid sensor molecule to be active. The interaction of the signaling agent with a nucleic acid sensor molecule may result in modification of the enzymatic nucleic acid component of the nucleic acid sensor molecule via chemical, physical, topological, or conformational changes to the structure of the molecule, such that the activity of the enzymatic nucleic acid component of the nucleic acid sensor molecule is modulated, for example is activated or deactivated. Signaling agents can comprise target signaling molecules such as macromolecules, ligands, small molecules, metals and ions, nucleic acid molecules including but not limited to RNA and DNA or analogs thereof, proteins, peptides, antibodies, polysaccharides, lipids, sugars, microbial or cellular metabolites, pharmaceuticals, and organic and inorganic molecules in a purified or unpurified form, or physical signals including magnetism, temperature, light, sound, shock, pH, capacitance, voltage, and ionic conditions.

By “enzymatic nucleic acid” is meant a nucleic acid molecule capable of catalyzing (altering the velocity and/or rate of) a variety of reactions including the ability to repeatedly cleave other separate nucleic acid molecules (endonuclease activity) or ligate other separate nucleic acid molecules (ligation activity) in a nucleotide base sequence-specific manner. Additional reactions amenable to nucleic acid sensor molecules include but are not limited to phosphorylation, dephosphorylation, isomerization, helicase activity, polymerization, transesterification, hydration, hydrolysis, alkylation, dealkylation, halogenation, dehalogenation, esterification, desterification, hydrogenation, dehydrogenation, saponification, desaponification, amination, deamination, acylation, deacylation, glycosylation, deglycosylation, silation, desilation, hydroboration, epoxidation, peroxidation, carboxylation, decarboxylation, substitution, elimination, oxidation, and reduction reactions on both small molecules and macromolecules. Such a molecule with endonuclease and/or ligation activity can have complementarity in a substrate binding region to a specified gene target, and also has an enzymatic activity that specifically cleaves and/or ligates RNA or DNA in that target. That is, the nucleic acid molecule with endonuclease and/or ligation activity is able to intramolecularly or intermolecularly cleave and/or ligate RNA or DNA and thereby inactivate or activate a target RNA or DNA molecule. This complementarity functions to allow sufficient hybridization of the enzymatic RNA molecule to the target RNA or DNA to allow the cleavage/ligation to occur. 100% complementarity is preferred, but complementarity as low as 50-75% can also be useful in this invention. In addition, nucleic acid sensor molecule can perform other reactions, including those mentioned above, selectively on both small molecule and macromolecular substrates, though specific interaction of the nucleic acid sensor molecule sequence with the desired substrate molecule via hydrogen bonding, electrostatic interactions, and Van der Waals interactions. The nucleic acids can be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme, finderon or DNA enzyme. All of these terminologies describe nucleic acid molecules with enzymatic activity.

There are several different structural motifs of enzymatic nucleic acid molecules that catalyze cleavage/ligations reaction, including but not limited to hammerhead motif, hairpin motif, hepatitis delta virus motif, G-cleaver motif, Amberzyme motif, inozyme motif, and Zinzyme motif.

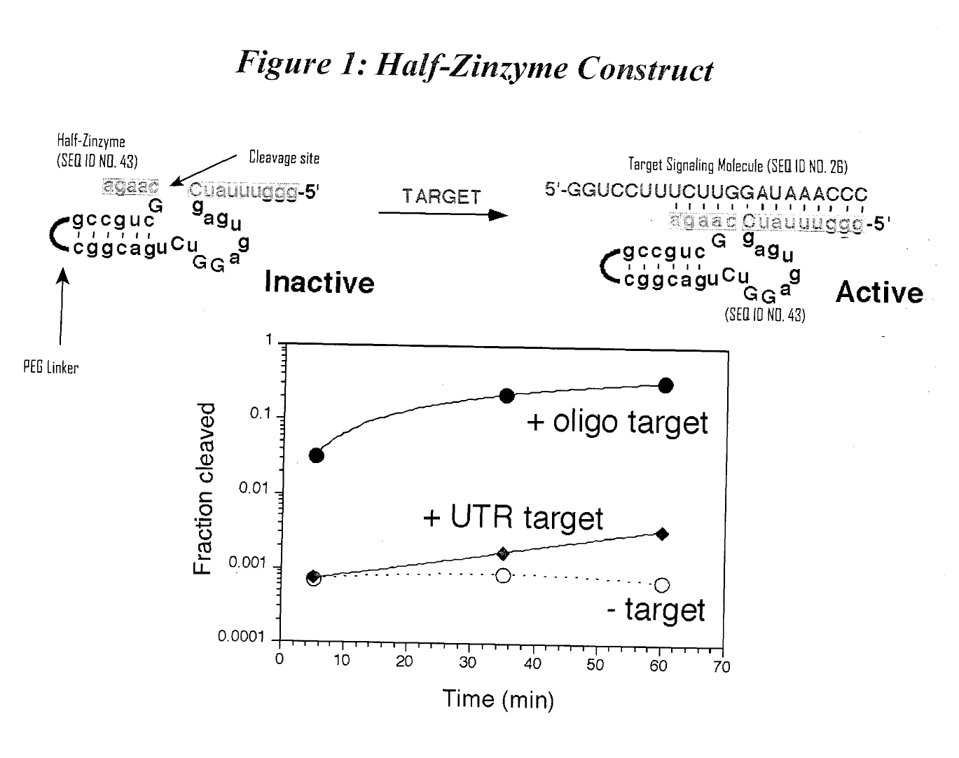

By “nucleic acid sensor molecule” as used herein is meant a nucleic acid molecule wherein the activity of the nucleic acid sensor molecule is modulated by the presence or absence of an effector molecule or target siganlling agent. Nonlimiting examples of nucleic acid sensor molecules of the invention are described in Usman et al., International PCT Publication No. WO 01/66721 incorporated by reference herein in its entirety including the drawings. In one embodiment, a nucleic acid sensor molecule or “multicomponent nucleic acid sensor molecule” as used herein refers to a nucleic acid sensor molecule assembled from one or more enzymatic nucleic acid domains (also referred to as “enzymatic nucleic acid components” herein) and one or more effector domains (also referred to as “effector components” herein). The multicomponent nucleic acid sensor molecule is active to catalyze a reaction involving a reporter molecule, when all the necessary components that make up the enzymatic nucleic acid domain interact with each other in a functional manner to catalyze the reaction. In one embodiment, the enzymatic nucleic acid domain is assembled from two or more separate nucleic acid molecules wherein the enzymatic nucleic acid domain is active only when all the separate nucleic acid molecules interact with each other in a manner necessary for the enzymatic nucleic acid domain to be active. In another embodiment, the enzymatic nucleic acid domain is made up of two separate nucleic acid molecules (also referred to as “Halfzyme” herein). In another embodiment, the enzymatic nucleic acid domain is assembled from two or more separate nucleic acids, wherein one of the separate nucleic acid molecules is the effector component. The reporter molecule can optionally be covalently attached to a portion of one of the nucleic acid sensor molecule components. The nucleic acid sensor molecule construct can be engineered such that the effector component of the multicomponent construct is provided in a sample or system of interest, i.e., in the absence of an appropriate effector component, the enzymatic nucleic acid component is unable to catalyze a reaction involving a reporter molecule. Whereas in the presence of the effector component, the enzymatic nucleic acid component and effector component are able to assemble into an active enzymatic nucleic acid molecule component (see for example FIG. 1) such that the nucleic acid sensor molecule is active to catalyze a reaction on a reporter molecule. He reaction catalyzed by the nucleic acid sensor molecule on a substrate does not cause any modification of the effector molecule. The effector and the reporter molecule are separate molecules.

By “effector component” or “effector molecule” or “effector” as used herein is meant any nucleic acid, polynucleotide, or oligonucleotide capable of interacting with an enzymatic nucleic acid component of a multicomponent nucleic acid sensor molecule in a manner that modulates the activity of the multicomponent nucleic acid sensor molecule. In one embodiment, the interaction of the effector component with the enzymatic nucleic acid component can provide a configuration such that the multicomponent nucleic acid sensor molecule is active in the presence of the effector. In another embodiment, the interaction of the effector component with the enzymatic nucleic acid component can also result in modification of the enzymatic nucleic acid component of the multicomponent nucleic acid sensor molecule via chemical, physical, topological, or conformational changes to the structure of the molecule, such that the activity of the enzymatic nucleic acid component of the nucleic acid sensor molecule is modulated, for example is activated or deactivated in the presence of the effector component. Effector components of the instant invention can comprise nucleic acid molecules indicative of infectious disease causing agents and/or markers of disease having a genetic basis. In another embodiment, the effector molecule interacts with the enzymatic nucleic acid component in the active site. In another embodiment, the effector molecule interacts with the enzymatic nucleic acid component in the allosteric site or site different from the active site. In another embodiment, the effector molecule makes up the active site or is an essential part of the active site of the enzymatic nucleic acid domain. The terms “effector component” or “effector” can also be referred to herein as “target nucleic acid” in the sense that the effector component can comprise a particular target nucleic acid molecule to be detected by the nucleic acid sensor molecule of the invention in a system, subject or sample. In one embodiment, the target nucleic acid comprises longer sequence than the effector component, for example wherein the effector component results from cleavage or processing of the target nucleic acid in a method of the invention.

By “enzymatic nucleic acid component” is meant a nucleic acid molecule capable of catalyzing (altering the velocity and/or rate of) a variety of reactions including the ability to repeatedly cleave other separate nucleic acid molecules (endonuclease activity) or ligate other separate nucleic acid molecules (ligation activity) in a nucleotide base sequence-specific manner in the presence of an effector component. An enzymatic nucleic acid component with endonuclease and/or ligation activity can have complementarity in a substrate binding region to a specified reporter molecule, and also has an enzymatic activity that specifically cleaves and/or ligates RNA or DNA reporter molecules. That is, the enzymatic nucleic acid component with endonuclease and/or ligation activity is able to intramolecularly or intermolecularly cleave and/or ligate RNA or DNA and thereby inactivate or activate a target RNA or DNA molecule in the presence of an effector component. This complementarity functions to allow sufficient hybridization of the enzymatic nucleic acid component to the reporter molecule to allow the cleavage/ligation to occur. 100% complementarity is preferred, but complementarity as low as 50-75% can also be useful in this invention.

The nucleic acids components and reporter molecules of the invention can be modified at the base, sugar, and/or phosphate groups.

By “substrate binding arm” or “substrate binding domain” or “substrate binding region” is meant that portion or region of a nucleic acid sensor molecule which is able to interact, for example, via complementarity (i.e., able to base-pair with), with a portion of its substrate or reporter. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). That is, these arms contain sequences within a nucleic acid sensor molecule which are intended to bring the nucleic acid sensor molecule and the target signaling molecule, for example RNA, together through complementary base-pairing interactions. The nucleic acid sensor molecule of the invention can have binding arms that are contiguous or non-contiguous and can be of varying lengths. The length of the binding arm(s) are preferably greater than or equal to four nucleotides and of sufficient length to stably interact with the target RNA. Preferably, the binding arm(s) are 12-100 nucleotides in length. More preferably, the binding arms are 14-24 nucleotides in length (see, for example, Werner and Uhlenbeck, supra; Hamman et al., supra; Hampel et al., EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “enzymatic portion” or “catalytic domain” is meant that portion or region of the nucleic acid sensor molecule essential for catalyzing a chemical reaction, such as cleavage of a nucleic acid substrate.

By “system” or “sample” is meant material, in a purified or unpurified form, from biological or non-biological sources, including but not limited to human, animal, soil, food, water, or others sources that comprise the effector component to be detected or amplified. As such, nucleic acid sensor molecules of the invention can be used to assay target compounds in biologic and non-biologic systems, such as in human and animal subjects or in samples of unidentified materials outside of a biological system.

The “biological system” or “biological sample” as used herein can be a eukaryotic system or a prokaryotic system, for example a bacterial cell, plant cell or a mammalian cell, or of plant origin, mammalian origin, yeast origin, Drosophila origin, or archebacterial origin.

By “reporter molecule” is meant a molecule, such as a nucleic acid sequence (e.g., RNA or DNA or analogs thereof) or peptides and/or other chemical moieties, able to stably interact with the nucleic acid sensor molecule and function as a substrate for the nucleic acid sensor molecule. The reporter molecule can be covalently linked to the nucleic acid sensor molecule or a portion of one of the components of a halfzyme. The reporter molecule can also contain chemical moieties capable of generating a detectable response, including but not limited to, fluorescent, chromogenic, radioactive, enzymatic and/or chemiluminescent or other detectable labels that can then be detected using standard assays known in the art. The reporter molecule can also act as an intermediate in a chain of events, for example, by acting as an amplicon, inducer, promoter, or inhibitor of other events that can act as second messengers in a system.

In one embodiment, the reporter molecule of the invention is an oligonucleotide primer, template, or probe, which can be used to modulate the amplification of additional nucleic acid sequences, for example, sequences comprising reporter molecules, target signaling molecules, effector molecules, inhibitor molecules, and/or additional nucleic acid sensor molecules of the instant invention.

By “sensor component” or “sensor domain” of the nucleic acid sensor molecule is meant, a molecule such as a nucleic acid sequence (e.g., RNA or DNA or analogs thereof), peptide, or other chemical moiety which can interact with one or more regions of a target signaling agent or more than one target signaling agents, and which interaction causes the enzymatic nucleic acid component of the nucleic acid sensor molecule to modulate, such as inhibit or activate, the catalytic activity of the nucleic acid sensor molecule. In the presence of a signaling agent, the ability of the sensor component, for example, to modulate the catalytic activity of the enzymatic nucleic acid component is inhibited or diminished. The sensor component can comprise recognition properties relating to chemical or physical signals capable of modulating the enzymatic nucleic acid component via chemical or physical changes to the structure of the nucleic acid sensor molecule. The sensor component can be derived from a naturally occurring nucleic acid protein binding sequence, for example RNAs that bind viral proteins such as HIV trans-activation response (TAR), HIV nucleocapsid, TFIIA, rev, rex, Ebola VP35, HCV core proteins, HBV core proteins; RNAs that bind eukaryotic proteins such as protein kinase R (PKR), ribosomal proteins, RNA polymerases, and ribonucleoproteins. The sensor component can also be derived from a nucleic acid sequence that is obtained through in vitro or in vivo selection techniques as are know in the art. Alternately, the sensor component can be derived from a nucleic acid molecule (aptamer) which is evolved to bind to a nucleic acid sequence within a target nucleic acid molecule. Such sequences or “aptamers” can be designed to bind a specific protein, peptide, nucleic acid, co-factor, metabolite, drug, or other small molecule with varying affinity. The sensor component can be covalently linked to the nucleic acid sensor molecule, or can be non-covalently associated. A person skilled in the art will recognize that all that is required is that the sensor component is able to selectively inhibit the activity of the nucleic acid sensor molecule.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, ligation, isomerization, phosphorylation, or dephosphorylation. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “alkyl” group is meant a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) are preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) can be preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH. Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalentlyjoined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1 -methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyunridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “unmodified nucleotide” is meant a nucleotide with one of the bases adenine, cytosine, guanine, thymine, uracil joined to the 1′ carbon of beta-D-ribo-furanose.

By “modified nucleotide” is meant a nucleotide that contains a modification in the chemical structure of an unmodified nucleotide base, sugar and/or phosphate.

By “unmodified nucleoside” is meant a nucleoside with one of the bases adenine, cytosine, guanine, thymine, uracil joined to the 1′ carbon of beta-D-ribo-furanose.

By “modified nucleoside” is meant a nucleotide that contains a modification in the chemical structure of an unmodified nucleoside base or sugar.

By “class I ligase” as used herein in meant an enzymatic nucleic acid molecule as generally described in Ekland et al., 1995, Science, 269, 364-370.

By “sufficient length” is meant an oligonucleotide of length sufficient to provide the intended function (such as binding) under the expected condition. For example, a binding arm of the enzymatic nucleic acid component of the nucleic acid sensor molecule should be of “sufficient length” to provide stable binding to the reporter molecule under the expected reaction conditions and environment to catalyze a reaction. In a further example, the sensor domain of the nucleic acid sensor molecule should be of sufficient length to interact with a target nucleic acid molecule in a manner that would cause the nucleic acid sensor to be active.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “nucleic acid molecule” as used herein is meant a molecule comprising nucleotides. The nucleic acid can be single, double, or multiple stranded and can comprise modified or unmodified nucleotides or non-nucleotides or various mixtures and combinations thereof. Nucleic acid molecules shall include oligonucleotides, ribozymes, DNAzymes, templates, and primers.

By “oligonucleotide” or “polynucleotide” is meant a nucleic acid molecule comprising a stretch of three or more nucleotides.

In a preferred embodiment the linker region, when present in the nucleic acid sensor molecule and/or reporter molecule is further comprised of nucleotide, non-nucleotide chemical moieties or combinations thereof. Non-limiting examples of non-nucleotide chemical moieties can include ester, anhydride, amide, nitrile, and/or phosphate groups.

In another embodiment, the non-nucleotide linker is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. W089/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. Thus, in a preferred embodiment, the invention features an nucleic acid sensor molecule of the invention having one or more non-nucleotide moieties, and having enzymatic activity to perform a chemical reaction, for example to cleave an RNA or DNA molecule.

By “cap structure” is meant chemical modifications which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples: the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein). In yet another preferred embodiment the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By “abasic” or “abasic nucleotide” is meant sugar moieties lacking a base or having other chemical groups in place of a base at the 1′ position, (for more details see Wincott et al., International PCT publication No. WO 97/26270).

The term “non-nucleotide” refers to any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenine, guanine, cytosine, uracil or thymine. The terms “abasic” or “abasic nucleotide” are meant to include sugar moieties lacking a base or having other chemical groups in place of a base at the 1′ position, (for more details see Wincott et al., International PCT publication No. WO 97/26270).

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “subject” is meant an organism, which is a donor or recipient of explanted cells or the cells themselves. “Subject” also refers to an organism to which the nucleic acid molecules of the invention can be administered. Preferably, a subject is a mammal or mammalian cells. More preferably, a subject is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the individual catalytic activity or stability of the nucleic acid molecule can be decreased (i.e., less than ten-fold), but the overall activity of the nucleic acid molecule is enhanced, in vivo.

By “nucleic acid circuit” or “nucleic acid-based circuit” is meant an electronic circuit comprising one or more nucleic acids or oligonucleotides.

By “nucleic acid computer” or “nucleic acid-based computer” is meant a computing device or system comprising one or more nucleic acids or oligonucleotides. The nucleic acid computer can be used to interface biological systems, control other devices, or can be utilized to solve problems and/or manipulate data. Furthermore, the nucleic acid computer may comprise nucleic acid circuits.

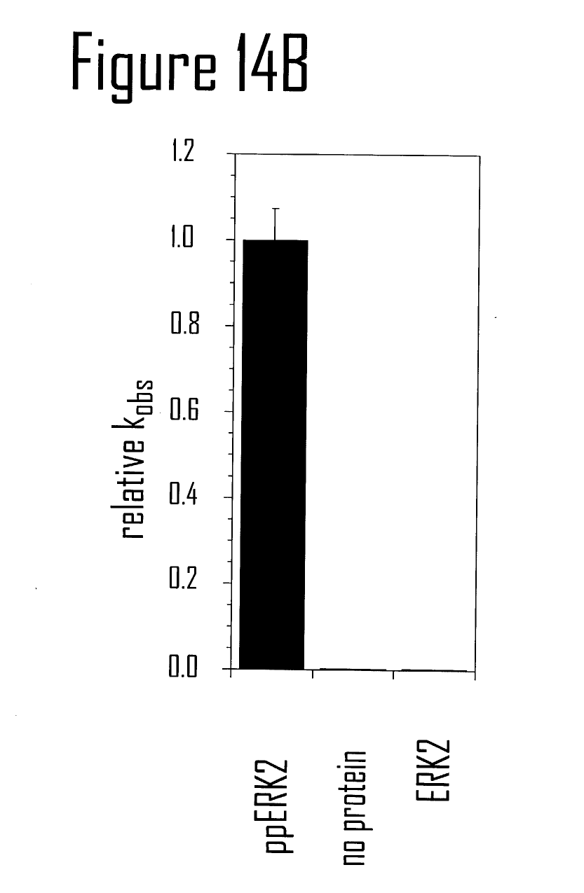

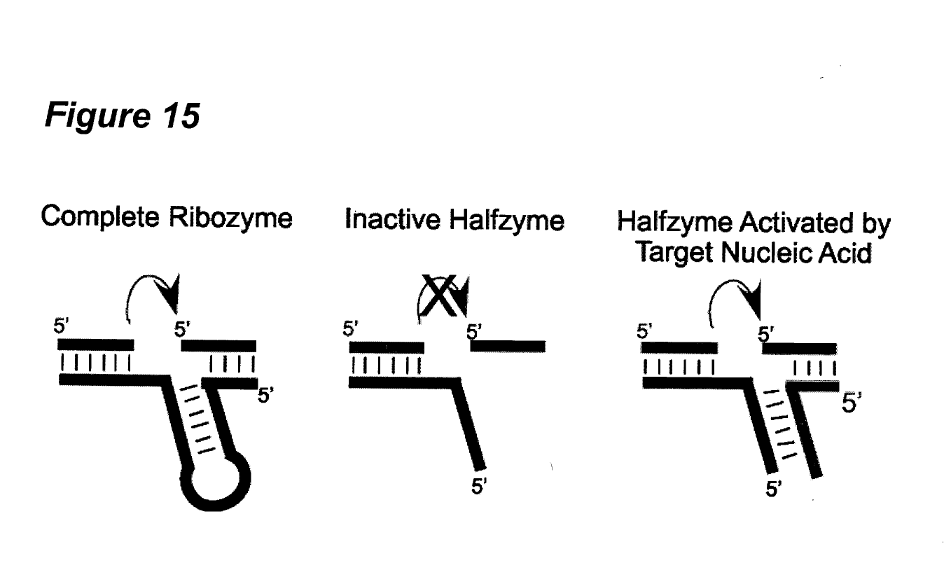

By “halfzyme” is meant an enzymatic nucleic acid molecule assembled from two or more nucleic acid components. The enzymatic nucleic acid in the halfzyme configuration is active to catalyze a reaction involving a reporter molecule, when all the necessary components that make up the enzymatic nucleic acid interact with each other. The reporter molecule may optionally be covalently attached to a portion of one of the halfzyme components. The halfzyme construct can be engineered such that an essential nucleic acid component of the enzymatic nucleic acid is provided by a target signaling agent of interest, i.e., in the absence of an appropriate target signaling agent the halfzyme construct is unable to catalyze a reaction involving a reporter molecule and in the presence of the target signaling agent, the halfzyme construct is able to assemble into an active enzymatic nucleic acid molecule (see for example FIGS. 1 and 15).

By “predetermined RNA molecule” is meant a particular RNA molecule of known sequence, such as a viral RNA, messenger RNA, transfer RNA, ribosomal RNA etc.

By “system” is meant a group of substances or components that can be collectively combined or identified. A system can comprise a biological system, for example an organism, cell, or components, extracts, and samples thereof. A system can further comprise an experimental or artificial system, where various substances or components are intentionally combined together.

By “detectable response” is meant a chemical or physical property that can be measured, including, but not limited to changes in temperature, pH, frequency, charge, capacitance, or changes in fluorescent, chromogenic, radioactive, enzymatic and/or chemiluminescent levels or properties that can then be detected using standard methods known in the art.

By “single stranded RNA” (ssRNA) is meant a naturally occurring or synthetic ribonucleic acid molecule comprising a linear single strand, for example a ssRNA can be a messenger RNA (mRNA), transfer RNA (tRNA), ribosomal RNA (rRNA) etc. of a gene.

By “single stranded DNA” (ssDNA) is meant a naturally occurring or synthetic deoxyribonucleic acid molecule comprising a linear single strand, for example, a ssDNA can be a sense or antisense gene sequence or EST (Expressed Sequence Tag).

By “predetermined target” is meant a signaling agent or target signaling agent that is chosen to interact with a nucleic acid sensor molecule to generate a detectable response.

By “validate a predetermined gene target” is meant to confirm that a particular gene is associated with a specific phenotype, disease, or biological function in a system. Once the relationship between a gene and its function or resulting phenotype is determined, the gene can be targeted to modulate the activity of the gene.

By “validate a predetermined RNA target” is meant to confirm that a particular RNA transcript of a gene or other RNA is associated with a specific phenotype, disease, or biological function in a system. Once the relationship between the RNA and its function or resulting phenotype is determined, the RNA can be targeted to modulate the activity of the RNA or the gene encoding the RNA.

By “validate a predetermined peptide target” is meant to confirm that a particular peptide is associated with a specific phenotype, disease, or biological function in a system. Once the relationship between the peptide and its function or resulting phenotype is determined, the peptide or RNA encoding the peptide can be targeted to modulate the activity of the peptide or the gene encoding the peptide.

By “validate a predetermined protein target” is meant to confirm that a particular protein is associated with a specific phenotype, disease, or biological function in a system. Once the relationship between the protein and its function or resulting phenotype is determined, the protein or RNA encoding the protein can be targeted to modulate the activity of the protein or the gene encoding the protein.

By “SNP” is meant a single nucleotide polymorphism as is known in the art to include single nucleotide substitutions or mismatches in a genome (see Brookes, 1999, Gene, 234, 177-186; Stephens, 1999, Molecular Diagnosis, 4, 309-317). SNPs can be used to identify genes and gene functions as well as to characterize a genotype.

By “validate a predetermined SNP target” is meant to confirm that a particular SNP of a gene is associated with a specific phenotype, disease, or biological function in a system. Once the relationship between the SNP and its function, associated gene function, or resulting phenotype is determined, the SNP can be targeted to modulate the activity of the SNP or the gene associated with the SNP.

By “SNP scoring” is meant a process of identifying and measuring the presence of SNPs in a genome. SNP scoring can also refer to a system of ranking single nucleotide polymorphisms in terms of the relationship between a particular SNP and a certain disease state or drug response in an organism, for example a human. SNP scoring can be used in determining the genotype of an organism.

By “proteome” is meant the complete set of proteins found in a particular system, such as a cell or organism, for example a human cell or human.

By “proteome map” is meant the functional relationship between different protein constituents of a proteome.

By “proteome scoring” is meant a process of identifying and measuring the presence of proteins in a proteome. Proteome scoring can also refer to a system of ranking protiens in terms of the relationship between a particular protein and a certain disease state or drug response in an organism, for example a human. Proteome scoring can be used in determining the phenotype of an organism.

By “disease specific proteome” is meant a proteome associated with a particular disease or condition.

By “treatment specific proteome” is meant a proteome associated with a particular treatment or therapy.

By “molecular profiling” is meant the use of nucleic acid sensor molecules for determining the prognosis and monitoring of human disease (e.g., cancer or human infectious disease) outcome in a subject and the use of nucleic acid sensor molecules for monitoring of subjects as a function of an approved drug or a drug under development, and/or subject surveillance and screening for drug and/or drug treatment. Molecular profiling as contemplated by the instant invention can comprise the use of profiling chip technology.

By “profiling chip” is meant a substrate to the surface of which one or more nucleic acid sensor molecules or components thereof are immobilized in a spatially defined and physically addressable manner (also referred to as arrays), for example wherein each nucleic acid sensor molecule immobilized on the substrate is designed to be dependent on a specific target protein co-factor. Each such Target Protein co-factor can be a marker for a specific human disease or condition (e.g., cancer or infectious disease). The profiling chip may be designed for a specific cancer or infectious disease or a group of related or unrelated cancers or infectious diseases. The nucleic acid sensor molecules can be immobilized to the surface of the substrate by means of in situ synthesis or via linkers. In certain embodiments, the term “profiling chip” shall include an individual chip array or a wafer containing more than one chip array, where the arrays are distinctly separated from each other.

In certain embodiment, the term “target protein” refers to a protein which can act as the co-factor for nucleic acid sensor molecule activity.

Other features and advantages of the invention will be apparent from the following description of the preferred embodiments thereof, and from the claims.

### Detection of Target Signaling Molecules

In one embodiment, the invention features several approaches to detecting signaling agents, ligands and/or target signaling molecules in a system using nucleic acid molecules. In all cases, activity of the nucleic acid is modulated via interaction of the nucleic acid with the target signaling agent, ligand and/or target signaling molecule.

In one embodiment, the present invention utilizes at least three oligonucleotide sequences for proper function: nucleic acid sensor molecule, reporter molecule, and target signaling molecule. In a non-limiting example, the nucleic acid sensor molecule is comprised of a sensor component and an enzymatic nucleic acid component. The nucleic acid sensor molecule can be further comprised of a linker between the sensor component and the enzymatic nucleic acid component. The nucleic acid sensor molecule is in its inactive state when the sensor component binds to the nucleic acid sensor molecule in the enzymatic nucleic acid component. The sensor component can bind to the substrate binding regions or nucleotides that contribute to the secondary or tertiary structure of the enzymatic nucleic acid component. For example, the sensor component can bind to nucleotides located within the nucleic acid sensor molecule, which can disrupt catalytic activity. The reporter molecule may be able to bind to the nucleic acid sensor molecule, but a catalytic activity would be inhibited since the molecule is structurally inactive. Alternatively, the sensor component can bind to the substrate binding region(s) of the enzymatic nucleic acid component, which can prevent the reporter molecule from binding to the nucleic acid sensor molecule. The sensor component cannot be cleaved because the cleavage site would contain either a chemical modification which prevents cleavage or an inappropriate sequence. For example, hammerhead ribozymes need to have a NUH motif in the molecule to be cleaved (H is adenosine, cytidine, or uridine) for proper cleavage. By adding a guanosine at the H position in the RNA to be cleaved, cleavage can be inhibited.

In the presence of the target signaling molecule, the sensor component can disassociate from the enzymatic nucleic acid component and bind to the target signaling molecule preferentially. The sensor component can preferentially bind to the target signaling molecule which results in the formation of a more stable complex. For example, the sensor component can bind to more nucleotides on the target signaling molecule than on the nucleic acid sensor molecule. Binding to a larger number of nucleotides can have increased chemical stability and therefore is preferred over binding to a smaller number of nucleotides.

When the sensor component is bound to the target signaling molecule and the reporter molecule binds to the nucleic acid sensor molecule, a reaction can be catalyzed on the reporter molecule by the enzymatic nucleic acid component. For example, the reporter molecule can be cleaved. The cleavage event can then be detected by using a number of assays. For example, electrophoresis on a polyacrylamide gel would detect not only the full length reporter oligonucleotide but also any cleavage products that were created by the functional nucleic acid sensor molecule. The detection of these cleavage products indicate the presence of the target signaling molecule. In addition, the reporter molecule can contain a fluorescent molecule at one end which fluorescence signal is quenched by another molecule attached at the other end of the reporter molecule. Cleavage of the reporter molecule in this case results in the disassociation of the florescent molecule and the quench molecule, resulting in a signal. This signal can be detected and/or quantified by methods known in the art (for example see Nathan et al., U.S. Pat. No. 5,871,914, Birkenmeyer, U.S. Pat. No. 5,427,930, and Lizardi et al., U.S. Pat. No. 5,652,107, George et al., U.S. Pat. Nos. 5,834,186 and 5,741,679, and Shih et al., U.S. Pat. No. 5,589,332).

Alternatively, the sensor of the signaling molecule can comprise a separate oligonucleotide sequence.

Target Sites

Targets for useful nucleic acid sensor molecules can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468 and hereby incorporated by reference herein in totality. Rather than repeat the guidance provided in those documents here, below are provided specific examples of such methods, not limiting to those in the art. Nucleic acid sensor molecules to such targets are designed as described in those applications and synthesized to be tested in vitro and in vivo, as also described. Such nucleic acid sensor molecules can also be optimized and delivered as described therein.

Hammerhead, hairpin, Inozyme, Zinzyme, Amberzyme and DNAzyme-based nucleic acid sensor molecules are designed that can bind and are individually analyzed by computer folding (Jaeger et al., 1989 Proc. Natl. Acad. Sci. USA, 86, 7706; Denman, 1993, Biotechniques, 15, 1090) to assess whether the nucleic acid sensor molecule sequences fold into the appropriate secondary structure. Those nucleic acid sensor molecules with unfavorable intramolecular interactions between the binding arms and the catalytic core are eliminated from consideration. Varying binding arm lengths can be chosen to optimize activity. Generally, at least 5 bases on each arm are able to bind to, or otherwise interact with, the target RNA. Nucleic acid molecules of the differing motifs are designed to anneal to various sites in the mRNA message. The binding arms are complementary to the target site sequences described above.

Hammerhead, DNAzyme, NCH, amberzyme, zinzyme or G-Cleaver-based nucleic acid sensor molecule cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; Wincott et al., 1995 Nucleic Acids Res. 23, 2677-2684; and Caruthers et al., 1992, Methods in Enzymology 211,3-19.

Nucleic Acid Molecule Synthesis

The nucleic acid molecules of the invention, including certain nucleic acid sensor molecules, can be synthesized using the methods described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59. Such methods make use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table I outlines the amounts and the contact times of the reagents used in the synthesis cycle. Alternatively, syntheses at the 0.2 μmol scale can be done on a 96-well plate synthesizer, such as the PG2100 instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Cleavage from the solid support and deprotection of the oligonucleotide is typically performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA●3HF to provide a 1.4 M HF concentration) and heated to 65° C. After 1.5 h, the oligomer is quenched with 1.5 M NH₄HCO₃. Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA●3HF (0.1 mL) is added and the vial is heated at 65° C. for 15 min. The sample is cooled at −20° C. and then quenched with 1.5 M NH₄HCO₃. An alternative deprotection cocktail for use in the one pot protocol comprises the use of aqueous methylamine (0.5 ml) at 65° C. for 15 min followed by DMSO (0.8 ml) and TEA●3HF (0.3 ml) at 65° C. for 15 min. A similar methodology can be employed with 96-well plate synthesis formats by using a Robbins Scientific Flex Chem block, in which the reagents are added for cleavage and deprotection of the oligonucleotide.

For anion exchange desalting of the deprotected oligomer, the TEAB solution is loaded onto a Qiagen 500® anion exchange cartridge (Qiagen Inc.) that is prewashed with 50 mM TEAB (10 mL). After washing the loaded cartridge with 50 mM TEAB (10 mL), the RNA is eluted with 2 M TEAB (10 mL) and dried down to a white powder.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile. Alternatively, for oligonucleotides synthesized in a 96-well format, the crude trityl-on oligonucleotide is purified using a 96-well solid phase extraction block packed with C18 material, on a Bahdan Automation workstation.

The average stepwise coupling yields are typically >98% (Wincott et al., 1995 Nucleic Acids Res. 23, 2677-2684). Those of ordinary skill in the art will recognize that the scale of synthesis can be adapted as larger or smaller than the example described above including but not limited to 96 well format, all that is important is the ratio of chemicals used in the reaction.

To ensure the quality of synthesis of nucleic acid molecules of the invention, quality control measures are utilized for the analysis of nucleic acid material. Capillary Gel Electrophoresis, for example using a Beckman MDQ CGE instrument, can be ulitized for rapid analysis of nucleic acid molecules, by introducing sample on the short end of the capillary. In addition, mass spectrometry, for example using a PE Biosystems Voyager-DE MALDI instrument, in combination with the Bohdan workstation, can be utilized in the analysis of oligonucleotides, including oligonucleotides synthesized in the 96-well format.

The nucleic acids of the invention can also be synthesized in two parts and annealed to reconstruct the nucleic acid sensor molecules (Chowrira and Burke, 1992 Nucleic Acids Res., 20, 2835-2840). The nucleic acids are also synthesized enzymatically using a variety of methods known in the art, for example as described in Havlina, International PCT publication No. WO 9967413, or from DNA templates using bacteriophage T7 RNA polymerase (Milligan and Uhlenbeck, 1989, Methods Enzymol. 180, 51). Other methods of enzymatic synthesis of the nucleic acid molecules of the invention are generally described in Kim et al., 1995, Biotechniques, 18, 992; Hoffman et al., 1994, Biotechniques, 17, 372; Cazenare et al., 1994, PNAS USA, 91, 6972; Hyman, U.S. Pat. No. 5,436,143; and Karpeisky et al., International PCT publication No. WO 98/28317).

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are preferably modified to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Nucleic acid sensor molecules are purified by gel electrophoresis using known methods or are purified by high pressure liquid chromatography (HPLC; See Wincott et al., Supra, the totality of which is hereby incorporated herein by reference) and are re-suspended in water.

Optimizing Nucleic Acid Molecule Activity

Synthesizing nucleic acid molecules with modifications (base, sugar and/or phosphate) that prevent their degradation by serum ribonucleases can increase their potency (see e.g., Eckstein et al., International Publication No. W092/07065; Perrault et al., 1990 Nature 344, 565; Pieken et al., 1991, Science 253, 314; Usman and Cedergren, 1992, Trends in Biochem. Sci. 17, 334; Usman et al., International Publication No. W093/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules described herein. All these references are incorporated by reference herein. Modifications which enhance their efficacy in cells, and removal of bases from nucleic acid molecules to shorten oligonucleotide synthesis times and reduce chemical requirements are preferably desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modifications of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated by reference herein in their totalities). Such publications describe general methods and strategies to determine the location of incorporation of sugar, base and/or phosphate modifications and the like into nucleic acid sensor molecule molecules without inhibiting catalysis. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in the presence of biological fluids, or in cells, the activity can not be significantly lowered. Clearly, nucleic acid molecules must be resistant to nucleases in order to function as effective diagnostic agents, whether utilized in vitro and/or in vivo. Improvements in the synthesis of RNA and DNA (Wincott et al., 1995 Nucleic Acids Res. 23, 2677; Caruthers et al., 1992, Methods in Enzymology 211,3-19; Karpeisky et al., International PCT publication No. WO 98/28317) (incorporated by reference herein) have expanded the ability to modify nucleic acid molecules by introducing nucleotide modifications to enhance their nuclease stability as described above.

In another aspect the nucleic acid molecules comprise a 5′ and/or a 3′-cap structure.

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Karpeisky et al., WO 98/28317, respectively, which are both incorporated by reference herein in their entireties.

Various modifications to nucleic acid (e.g., nucleic acid sensor molecule) structure can be made to enhance the utility of these molecules. Such modifications enhance shelf-life, half-life in vitro, stability, and ease of introduction of such oligonucleotides to the target site, e.g., to enhance penetration of cellular membranes, and confer the ability to recognize and bind to targeted cells.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995, which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819, all of which are incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a subject.

The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for exaple the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A.R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; Skillern et al., International PCT Publication No. WO 00/22113; Conrad, International PCT Publication No. WO 00/22114; and Conrad, U.S. Pat. No. 6,054,299; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, nucleic acid molecules of the present invention are preferably expressed from transcription units (see for example Couture et al., 1996, TIG., 12, 510, Skillern et al., International PCT Publication No. WO 00/22113, Conrad, International PCT Publication No. WO 00/22114, and Conrad, U.S. Pat. No. 6,054,299) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intra-muscular administration, by administration to target cells ex-planted from the subject followed by reintroduction into the subject, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase H (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res.., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20,4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566; all of these references are incorporated by reference herein). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing techniques useful in isolating nucleic acid molecules of the instant invention.

### Example 1

Half-Zinzyme Nucleic Acid Sensor Molecule (Halfzyme)

Applicant has developed a generalizable methodology for the production of nucleic acid sensor molecules that are activated by target nucleic acids. This technology is based on enzymatic nucleic acids that, in the absence of a target nucleic acid, are catalytically inactive because they lack portions of the catalytic core and substrate recognition elements. In this ‘half-ribozyme' or ‘halfzyme' system, catalysis can occur if a specific target nucleic acid supplies the sequences required for catalysis in trans.

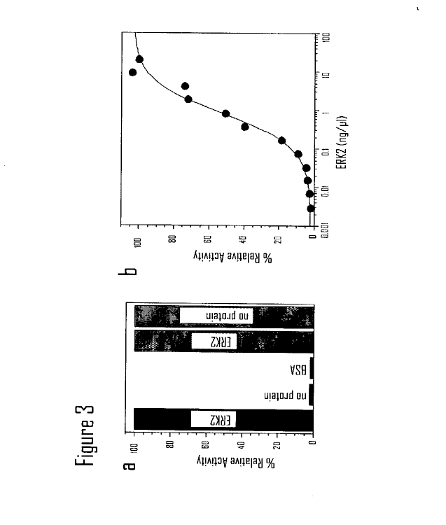

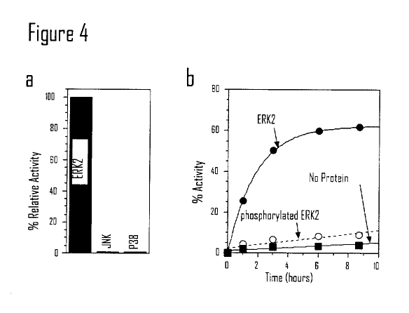

Although many enzymatic nucleic acid motifs can be used for the halfzyme strategy, one system uses the Zinzyme motif (FIG. 3) in which the substrate nucleic acid is attached to the enzymatic nucleic acid. This motif is small (about 32 nucleotides), carries modifications that confer a half-life in serum of greater than 100 hours, and has minimal target sequence requirements (5′-N3-RG-N3-3′, where N=any nucleotide and R=A or G). Thus, this motif is readily synthesized, has the ability to detect different sequences, and can be used directly in serum or other biological fluids.

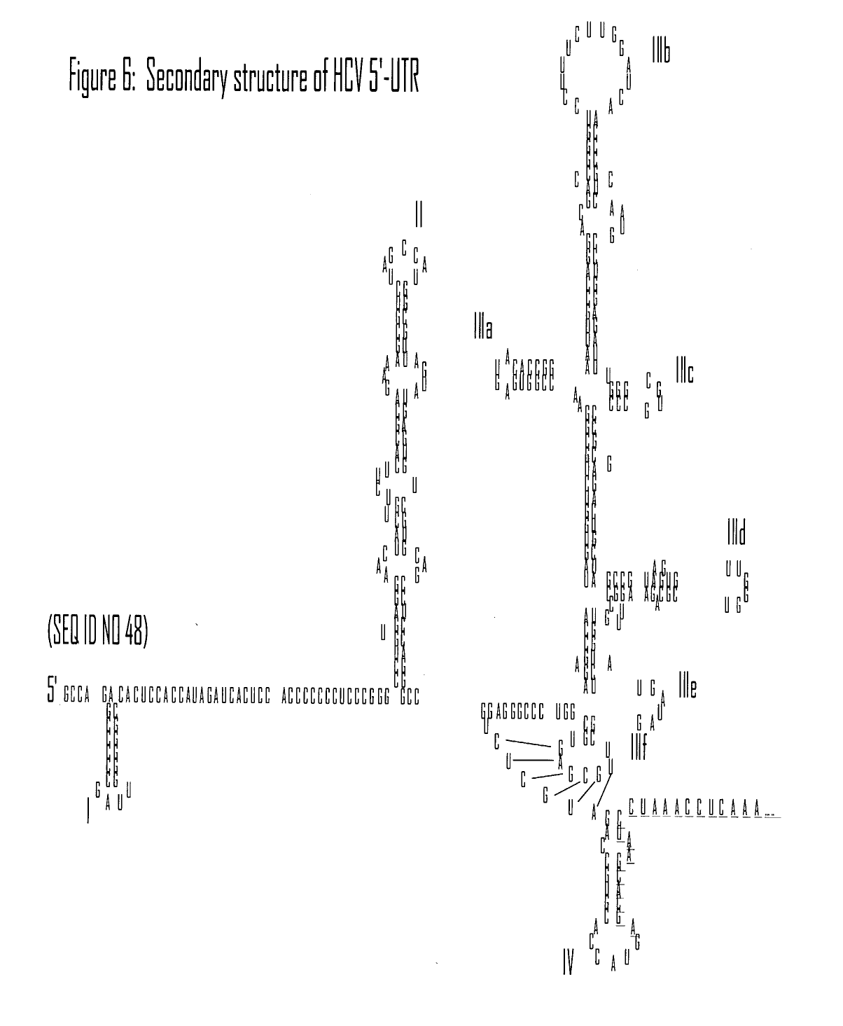

Applicant has tested the feasibility of the halfzyme approach using the Zinzyme motif and the Hepatitis C Virus genome as a model target. A synthetic oligoribonucleotide representing loop IIIB of the 5′ untranslated region (UTR), a universally conserved region of the HCV genome, activates catalysis of a rationally designed, sequence matched halfzyme. In the absence of oligoribonucleotide target no nucleic acid sensor molecule activity is detected. Other regions of the HCV 5′-UTR (see FIG. 6) can be similarly used in the design of other halfzymes contemplated by the invention.

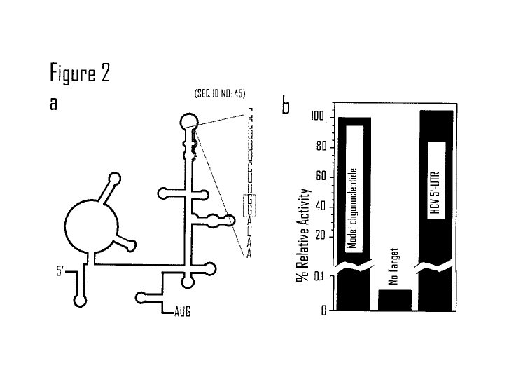

In this example, the halfzyme is activated by a target sequence derived from intact HCV genome. The 5′-UTR of HCV folds into a compact three-dimensional structure independent of the remaining portion of the HCV genome. To disrupt this structure so that UTR-derived loop IIIB sequences are accessible for activation of the halfzyme, a simple 20 minute pre-treatment step was inserted into the assay. Pre-treatment of the HCV 5′-UTR with a DNA oligonucleotide complementary to stem III and RNase H (FIG. 2a) is sufficient to activate halfzyme catalysis to the same extent as that observed with a short synthetic oligoribonucleotide (FIG. 2b). Thus, the halfzyme used in these studies can efficiently detect the presence of a conserved sequence element derived from the HCV genome. Target capture by a halfzyme is determined by the affinity of the halfzyme for its target and can be described in molarity by a dissociation constant. The value of this dissociation constant can be rationally engineered into the halfzyme, allowing 100% target capture when halfzyme used in the assay is in excess of this concentration.

A primary concern of any technology aimed at detecting low concentrations of nucleic acids is its sensitivity. The halfzyme approach is unique because catalysis is only promoted in the presence of a sequence-matched target and because 100% target capture can be achieved by manipulating halfzyme concentration. Therefore, single molecule detection is theoretically possible by this approach provided that an adequate signal amplification system is in place. Given the enormous flexibility of possible signal amplification and detection systems accommodated by the technology, signal detection should not define the limit of sensitivity of this technology. In practice, the limit of sensitivity of this approach is dictated by the uncatalyzed rate of substrate cleavage promoted under the assay conditions used. Therefore, the salient issue in terms of sensitivity becomes the relative rate of catalyzed versus uncatalyzed substrate cleavage. A virtue of the system is that both the assay conditions and halfzyme activity can be manipulated to maximize this rate differential.

FIG. 1 shows a non-limiting example of a “half-zinzyme” nucleic acid sensor molecule with a PEG linker that is modulated by the 5′-UTR of the Hepatitis C virus (HCV 5′-UTR). The figure shows both inactive and active forms of the zinzyme sensor molecule (SEQ ID NO. 43). In the presence of the target signaling oligonucleotide (SEQ ID NO. 26) which represents the stem loop IIIB of the HCV 5′-UTR, the zinzyme sensor demonstrates an activity increase of three logs in cleaving the reporter molecule component of the sensor molecule as shown in the graph (+ oligo target) as compared to the sensor molecule in the absence of the target. In the presence of the full length 350 nt. HCV 5′-UTR, the zinzyme sensor molecule demonstrates an almost one log increase in activity in cleaving the reporter molecule component of the sensor molecule. Reaction conditions: 140 mM KCl, 10 mM NaCl, 20 mM HEPES pH 7.4, 1 mM MgCl2, 1 mM CaCl2, 400 nM Nucleic acid sensor, 400 nM Target, Trace of labeled reporter (˜10 nM), 25 μl reaction volume, Nucleic acid sensor, target and reporter were heated at 75° C. for 3 min, cooled to 37° C. and cleavage initiated by the addition of MgCl2 and CaCl2.

### Example 2

Nucleic Acid Sensor Ligase

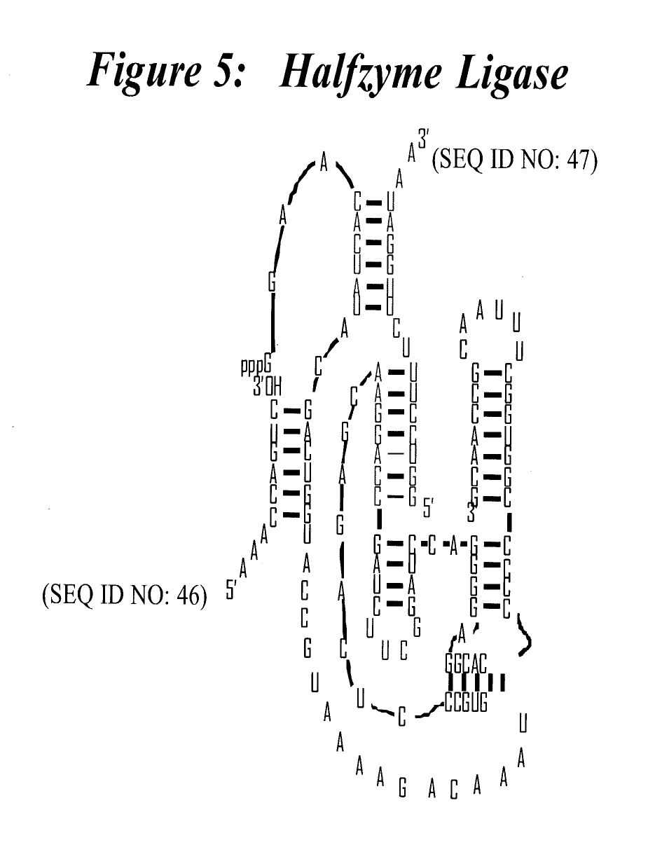

A ligase derived from the Bartel class I ligase (Ekland et al., 1995, Science, 269, 364-370) was prepared. Three different constructs carried various 3′ truncations. These segments were supplied in trans as oligonulceotide HCV sequence. One ligase, termed HZBART-2 showed ligation rate 107 fold above background ligation (FIG. 5).

Ligation reactions were performed at room temperature in 30 mM Tris, pH 7.5, 200 mM KCl, 60 mM MgCl2 and 0.6 mM EDTA. Halfzyme ligases (1 μM) with corresponding effector oligonulceotide (1 μM) were heated in water at 90° C. for 2 min and cooled at room temperature for 10 min followed by the addition of salt, buffer and 32P-labeled substrate oligonucleotide (0.1 mM final concentration). Reactions were carried out for 60 min at room temperature and stopped by the addition of 1 volume of gel loading buffer (7M urea, 100 mM EDTA) and snap cooling on ice. Products were separated on 20% denaturing polyacrylamide gel electrophoresis.

### Example 11

Halfzyme SNP Discrimination

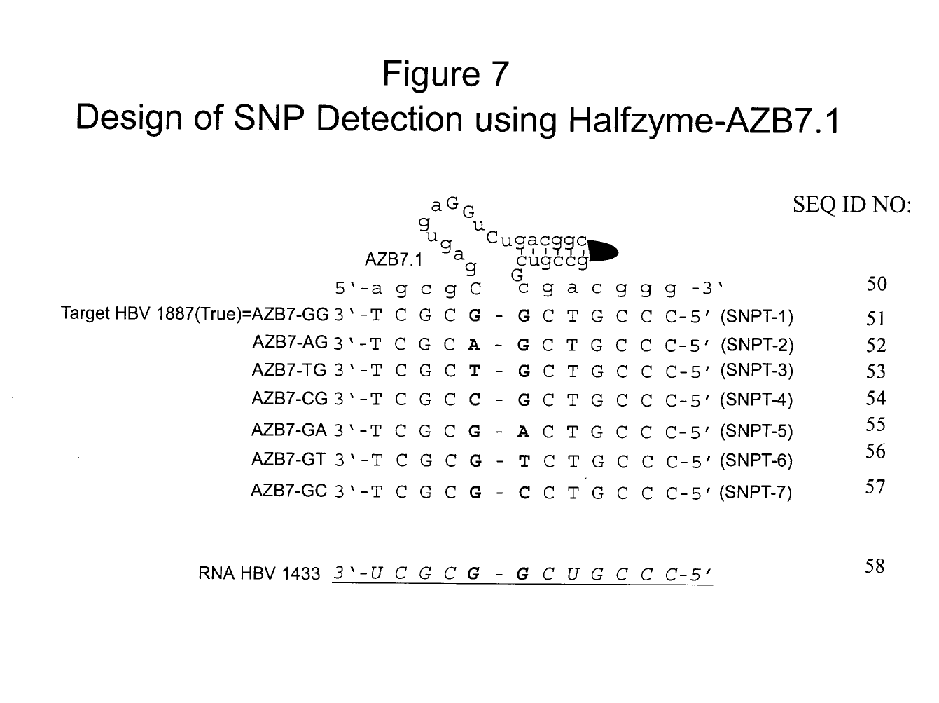

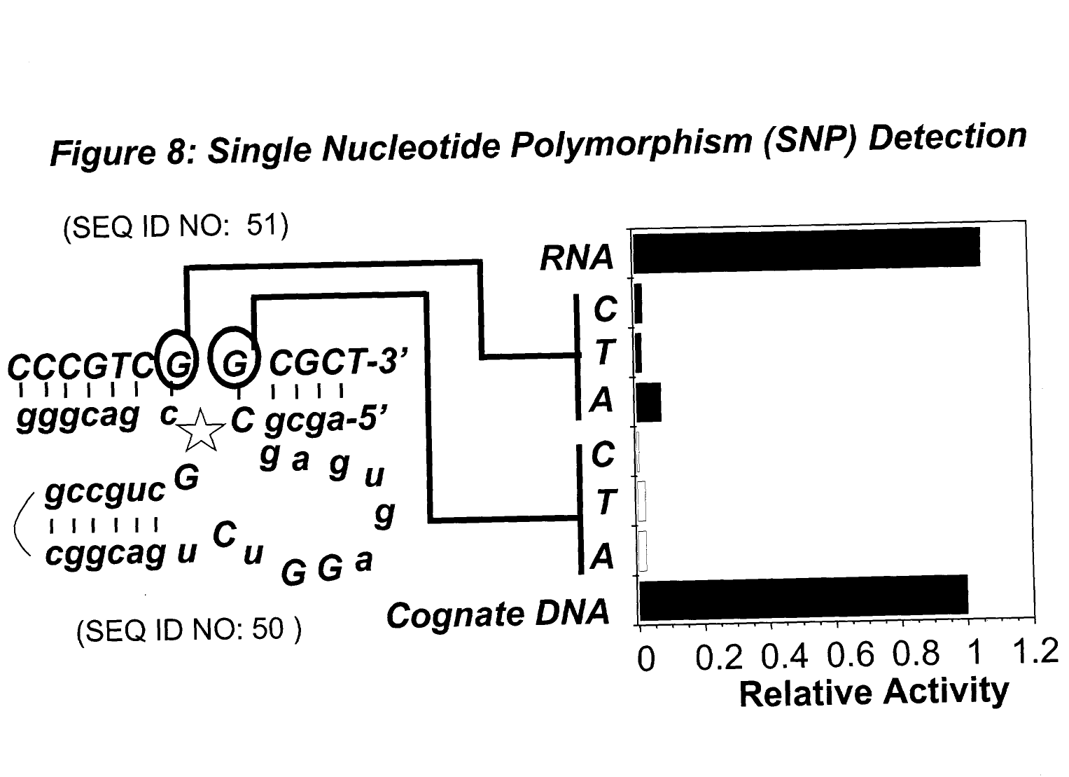

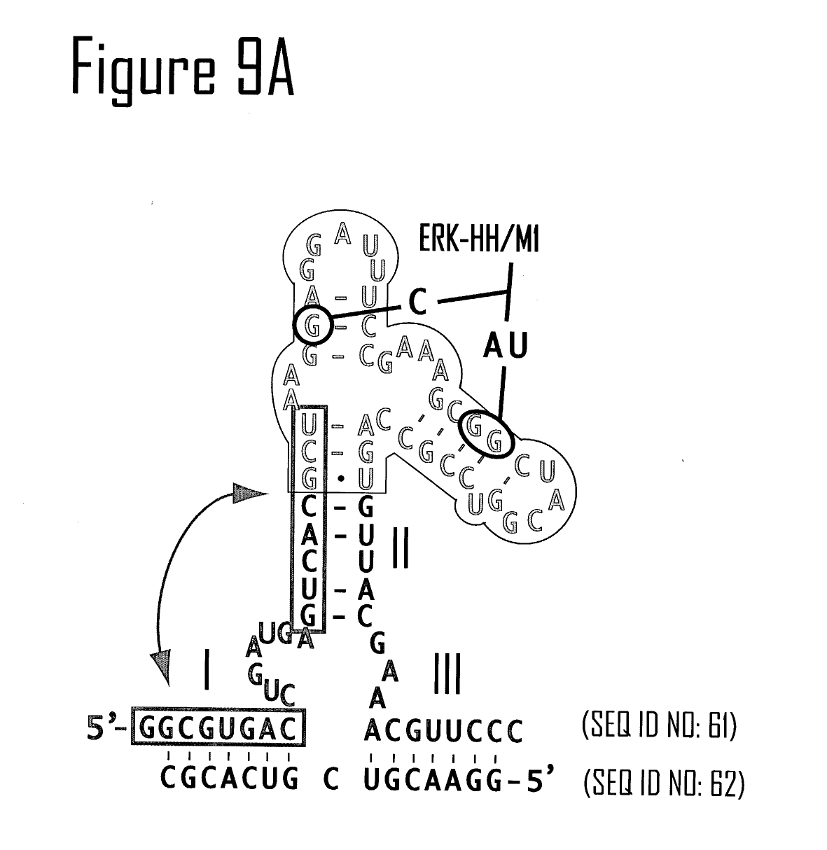

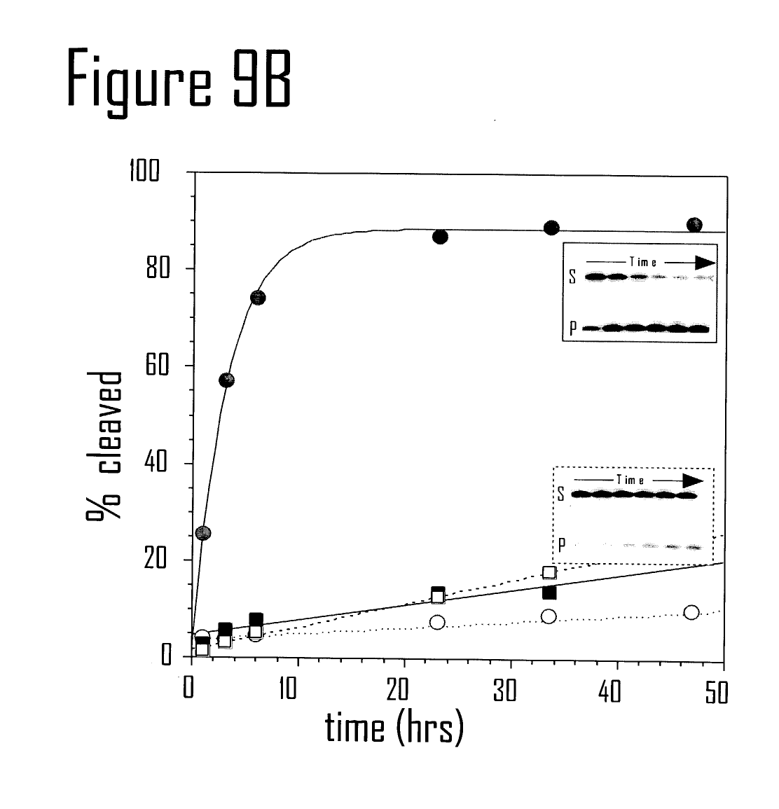

A halfzyme, based on a zinzyme enzymatic nucleic acid motif, (AZB7.1) was used to discriminate single nucleotide polymorphisms in a nucleic acid sequence derived from HBV (for example GenBank Accession No. AF100308.1). The design of the halfzyme and the sequences used for detecting single nucleotide substitutions within a target sequence are shown in FIG. 7. The cognate HBV DNA sequence used contains the sequence 3′-TCGCGGCTGCCC-5′ (SEQ ID NO: 51). Two deoxy-guanosine nucleotides within the cognate sequence were each systematically replaced with alternate deoxy nucleotides (c, t, or a) and cleavage activity of the halfzyme (SEQ ID NO: 50) assessed for each single nucleotide substitution in the target sequence. As shown in FIG. 8, efficient halfzyme cleavage takes place in the presence of the cognate DNA sequence (SEQ ID NO: 51) and a corresponding all RNA sequence (HBV 1433, SEQ ID NO: 58). However, the introduction of single nucleotide changes within the target sequence (SEQ ID NOS: 52-57) results in loss of cleavage activity at both positions tested within the sequence. This study demonstrates that nucleic acid sensor molecules of the invention, specifically halfzymes, can be used to detect single nucleotide polymorphisms in a target nucleic acid sequence.

Each reaction of the study contained a certain amount of DNA target to be analyzed, 10 uM of ³²P labeled halfzyme AZB7.1 in 10 ul of 1× Buffer (20 mM MES pH6.0, 14 mM KCl and 10 mM NaCl) with 10 ng/ul Monkey Genomic DNA and 1 mM CaCl₂ and 1 mM MgCl₂. Reactions were assembled with all components except the CaCl₂ and MgCl₂, heated to 80° C. for 5 mins, then cooled to 32° C. slowly. The reactions were initiated with the addition of the CaCl₂ and MgCl₂, and incubated overnight. The reactions were terminated by the addition of 10 ul of XC/BPB loading dye. The products were resolved by electrophoresis through a 15% denaturing polyacrylamide gel (19:1 cross link) with 7M urea in 1× TBE buffer. The gel was visualized by phosphoimager analysis.

### Example 12

Monitoring Post-Translational Modification of Proteins in Solution with Nucleic Acid Sensor Molecules

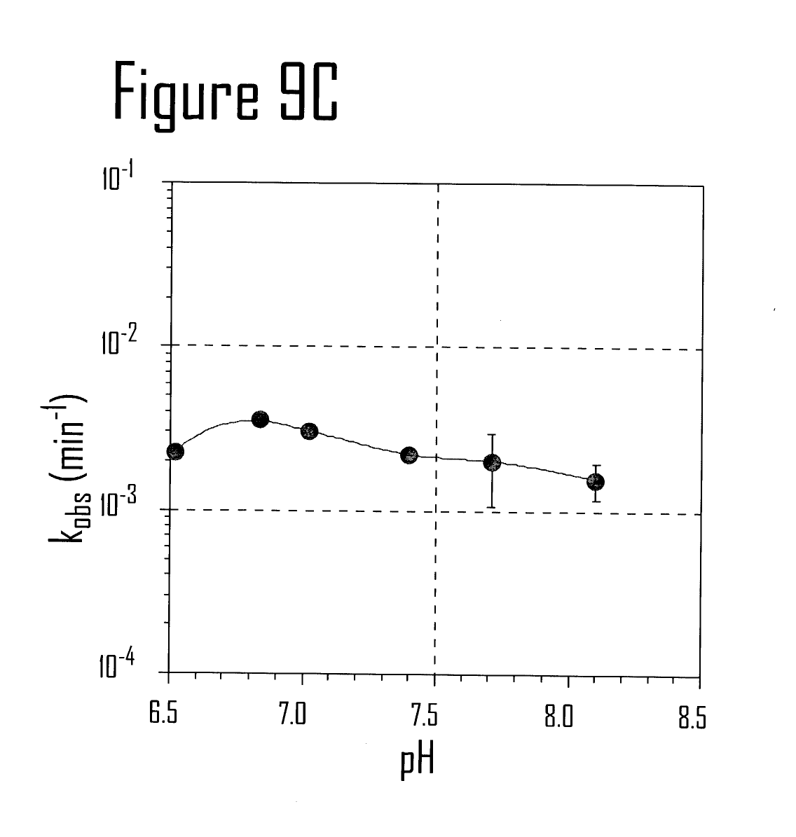

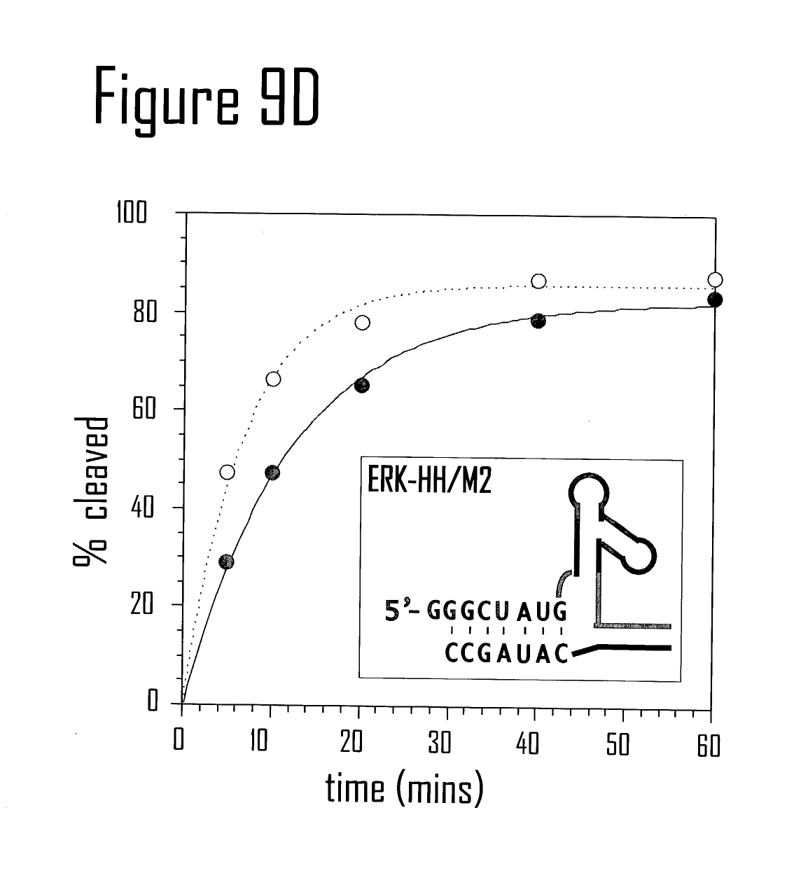

A pre-existing RNA ligand specific for the unphosphorylated form of ERK2 was linked to a variant of the hammerhead ribozyme through a destabilized stem II structure (ERK-HH, FIG. 9A). Biochemical and structural studies have demonstrated that activity of the hammerhead ribozyme motif requires formation of stem II. Consequently, a reasonable strategy is to induce formation of stem II through molecule binding to an appended RNA ligand. Protein binding can serve to induce ribozyme activity by stabilizing stem II since association of ERK2 with the RNA ligand requires at least partial formation of stem II in the fusion construct. To further disfavor stem II formation in the absence of ERK2, a substrate RNA binding arm in ERK-HH was made complementary to sequences in the destabilized stem II structure in order to form an alternate ERK-HH conformer incapable of cleaving substrate RNA (boxed regions, FIG. 9A). Upon ERK2 association, this alternate pairing arrangement should be prohibited and substrate RNA, such as a reporter molecule, can therefore associate with, and consequently be cleaved by, ERK-HH.

Nucleic acid sensor molecule activity assays were performed in the presence or absence of ERK2 to assess protein-dependent nucleic acid sensor molecule activation. Cleavage reactions contained 10 mM Tris-HCl, pH 7.5, 10 mM MgCl₂, 0.05 μg/μl tRNA, 100 nM nucleic acid sensor molecule, 500 nM protein (or the concentration indicated), and trace 5′-P³²-labeled substrate RNA. Recombinant rat ERK2 was produced and purified as described in Golden et al., 2000, J. Biotechnol., 81, 167. In all reactions, the level of substrate RNA cleavage in the absence of nucleic acid sensor molecule was subtracted from the level of substrate RNA cleavage in the presence of nucleic acid sensor molecule. Data represent the average values from two or more experiments. ERK-HH displayed little activity in the absence of the ERK2 protein (k_{obs}=4.2×10⁻⁵ min⁻¹) (FIG. 9B). However, unphosphorylated ERK2 stimulated the observed rate by approximately 50 fold (k_{obs+ERK2}=2.1×10⁻³ min⁻¹). The observed rate of substrate RNA cleavage by ERK-HH in the presence of ERK2 did not display a log-linear relationship with pH but rather was independent of pH (FIG. 9C), suggesting that a conformational rearrangement of the enzymatic nucleic acid domain is the rate-limiting step in product formation. Importantly, catalysis promoted by a nucleic acid sensor molecule containing a mutated RNA ligand that does not associate with ERK2 was unaffected by the presence of ERK2, and was equivalent to the activity of ERK-HH in the absence of ERK2 (ERK-HH/M1, FIG. 9B). Thus, the catalytic activation of ERK-HH in the presence of ERK2 results from its capacity to recognize ERK2.

The rational design strategy used to create ERK-HH differs from previous allosteric ribozyme design strategies in that it employs ERK2-modulated sequestration of a substrate RNA binding element (boxed region, FIG. 9A). To determine the importance of this novel design element, a version of ERK-HH was constructed such that the sequences in stem I are unable to interact with stem II sequences (ERK-HH/M2; inset in FIG. 9D). The appropriate substrate (reporter molecule) for this nucleic acid sensor molecule was cleaved at nearly the same rate and to nearly the same extent in the presence or absence of ERK2 (FIG. 9D), suggesting that this design element plays a dominant role in protein-mediated activation of ERK-HH. Interestingly, the observed rate of substrate RNA cleavage promoted by ERK-HH/M2 was approximately twenty-fold greater than the ERK2-stimulated rate of ERK-HH. Thus, the rate-limiting conformational rearrangement of ERK-HH evidenced by the pH independence of substrate RNA cleavage (FIG. 9C) may involve the alternate pairing of stem regions I and II. The production of nucleic acid sensor molecules with an even greater rate induction by ERK2 may be accomplished by further engineering of ERK-HH to tune the protein dependence of this conformational rearrangement.

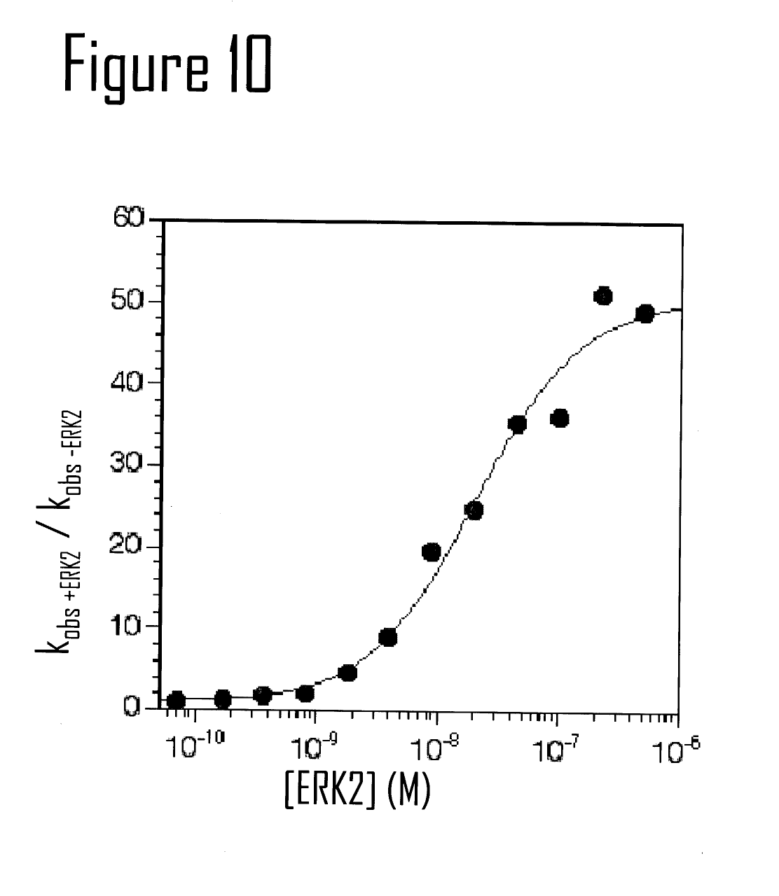

Importantly, ERK-HH activity was responsive to the concentration of ERK2 (FIG. 10). Maximal activation occurred in the presence of 500 nM ERK2, and activation was observed with as little as 5 nM ERK2 (FIG. 10). The ability of ERK-HH activity to monitor low nanomolar concentrations of ERK2 sets this nucleic acid sensor molecule apart from previously reported allosteric ribozymes, which respond to micromolar through millimolar concentrations of their cognate targets. This enhanced sensitivity reflects the use of an RNA ligand domain (sensor domain) in ERK-HH that displays nanomolar affinity for ERK2. Detection of even lower levels of protein target is possible through ‘affinity maturation’, a technique that has been used to increase the sensitivity of small molecule detection by allosteric ribozymes by over one hundred fold (Soukup et al., 2001, RNA, 7, 524). Alternatively, increased sensitivity of detection is possible by further increasing the rate differential between ERK2-stimulated and ERK2-indepdendent ERK-HH catalysis. Given that the proven detection limit of RNA reagents is equivalent to antibodies (Golden et al., 2000, J. Biotechnol., 81, 167), protein-activated nucleic acid sensor molecules therefore should ultimately prove to be useful alternatives to antibodies in certain applications.

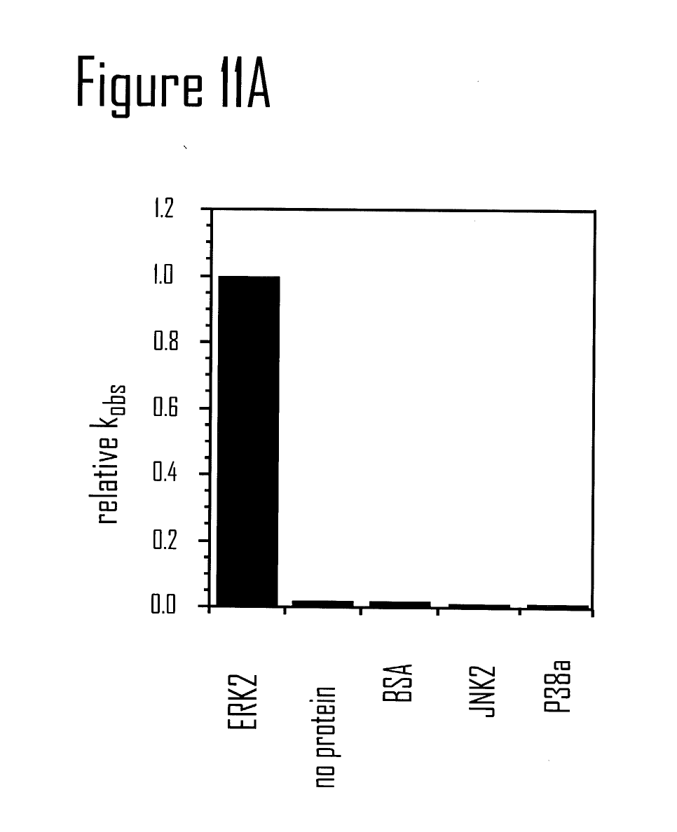

Since nucleic acid ligands developed through combinatorial methods can discriminate between protein isoforms and activation states, the specificity of protein-dependent ERK-HH activation was examined. As expected, bovine serum albumin (BSA) failed to activate ERK-HH above the level seen in the absence of any protein (FIG. 11A). More importantly, p38α and JNK2, MAPKs that are 45% similar to ERK2, failed to stimulate ERK-HH activity (FIGS. 3, 39A), demonstrating selectivity of this nucleic acid sensor molecule. Because RNA ligands can recognize conformational epitopes and because phosphorylation of ERK2 leads to kinase activation by promoting a conformational change, applicant examined whether ERK-HH was selectively activated by a specific phosphorylation state of ERK2. In contrast to unphosphorylated ERK2, phosphorylated ERK2 (T₁₈₃, Y₁₈₅-doubly phosphorylated ERK2; ppERK2) afforded minimal nucleic acid sensor molecule activation as judged by the low plateau level of cleavage in the presence of a molar excess of ppERK2 (FIG. 11B). Such phosphorylation-state specificity indicates that ERK-HH activation through a nonspecific RNA chaperone effect is unlikely. Analysis of ppERK2 by polyacrylamide gel electrophoresis demonstrates that approximately 10% of the ppERK2 preparation comprises unphosphorylated protein (inset, FIG. 11B); a percentage that correlated well with the relative plateau level of cleavage observed with ppERK2 (8.1%). Therefore, the low level of ERK-HH activity seen with the preparation of phosphorylated ERK2 most likely reports the small amount of contaminating unphosphorylated protein present in the ppERK2 preparation. Consequently, this protein-activated nucleic acid sensor molecule not only differentiates between ERK2 and MAPKs involved in other cellular processes, it also successfully monitors the post-translational activation state of ERK2.

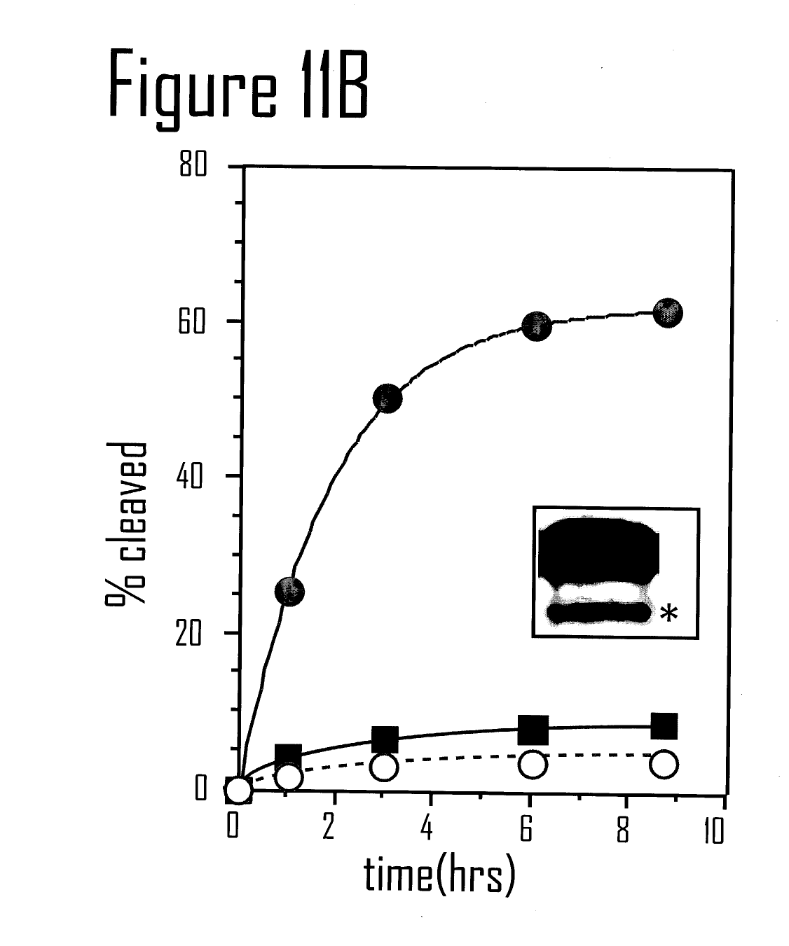

To serve as useful protein detection reagents, protein-activated nucleic acid sensor molecules should be able to detect their targets in complex mixtures of proteins. To examine this, ERK-HH was tested for its ability to monitor ERK2 in mammalian cell lysates. Exogenous ERK2 was added to aliquots of lysate to levels between 1% (500 nM) and 0.1% (50 nM) of the total protein by weight (FIG. 12A), concentrations of recombinant ERK2 that can be detected if purified (FIG. 10). ERK-HH faithfully reported the concentration of exogenous ERK2 in these samples with an activity that was reduced only two fold relative to its activation with purified ERK2 (FIG. 12B). Thus, these data demonstrate that a protein-activated nucleic acid sensor molecule can quantitatively detect its target in a complex mixture of cellular proteins and other macromolecules with only a slightly reduced capacity.

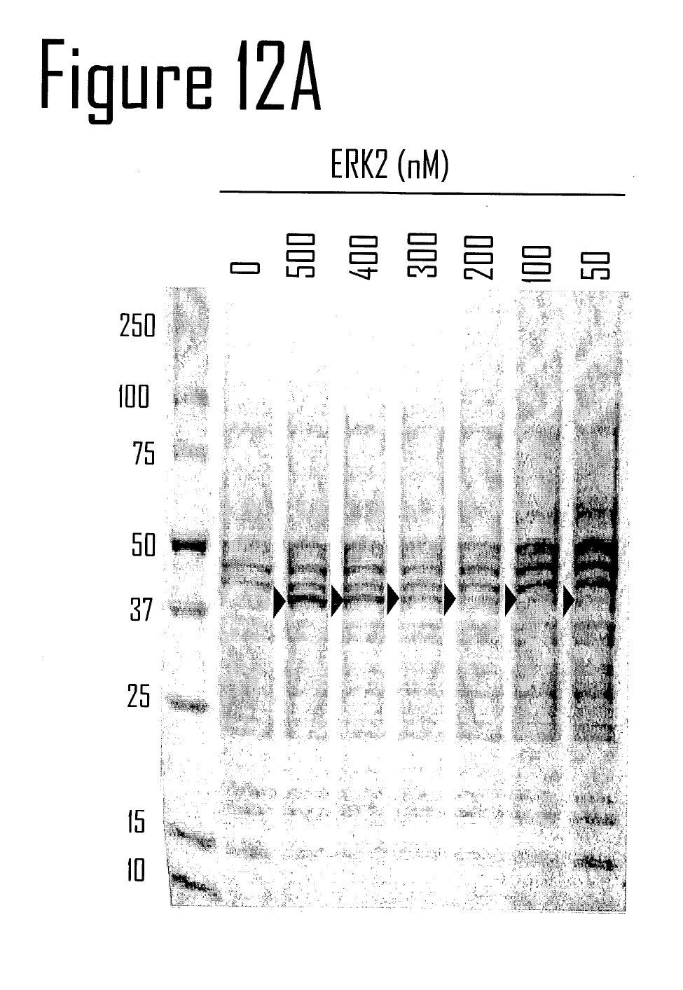

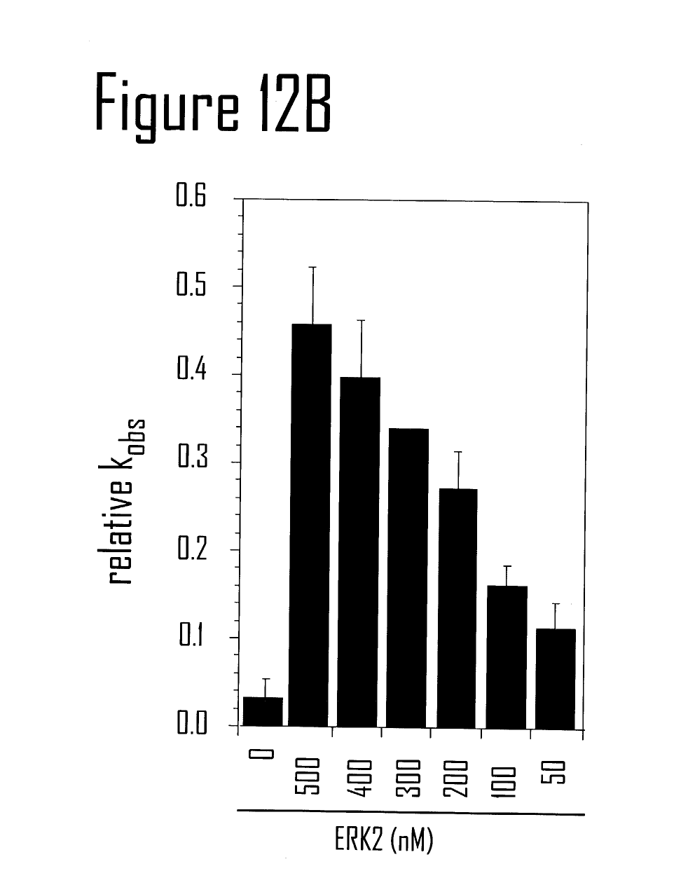

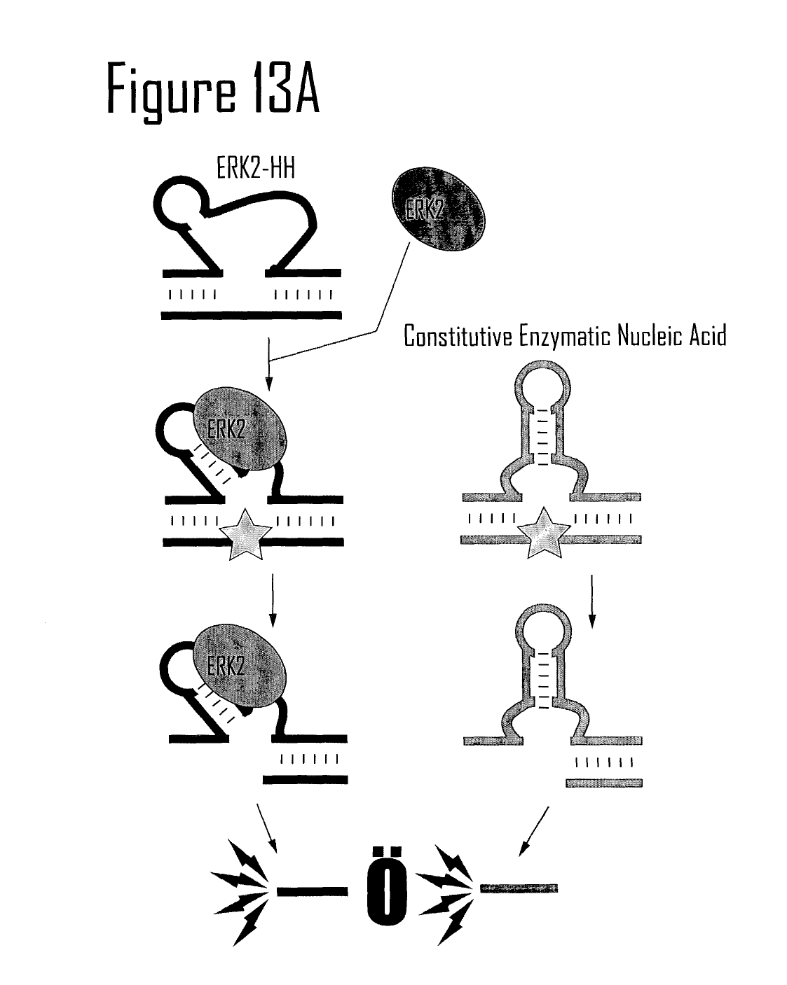

Because allosteric ribozymes couple analyte recognition and signaling in a single molecular event, we examined whether a protein-activated nucleic acid sensor molecule could monitor its target in a solution phase assay. To investigate whether a Fluorescence Resonance Energy Transfer (FRET)-based method could detect the activity of ERK-HH, an assay was developed in which ERK-HH separated a fluorescein dye from a fluorescein dye quencher that were coupled to opposite ends of a substrate RNA. The reactions were performed in 450 μl assays containing 100 nM substrate RNA for ERK-HH (5′-fluorescein-ggaacgUCGucacgc-BHQ-3′, SEQ ID NO: 59) and 100 nM substrate RNA for a constitutive ribozyme (5′-Cy3-ugagcUGcacugc-BHQ-3′, SEQ ID NO: 60) obtained from Integrated DNA Technologies, U.S.A. (lower case=2′-O-methyl ribonucleotide, BHQ=Black Hole Quencher™). Reaction conditions were identical to standard conditions described previously, except that sodium and potassium salts at final concentrations of 10 mM and 14 mM, respectively, were included; a requirement for activity of the constitutive ribozyme motif. Emission at 517 nm and 568 nm was measured during the initial rate phase of reactions (5.5 hours). A constitutive ribozyme that cleaved a substrate RNA carrying a similarly quenched cyanine 3 (Cy3) fluorophore was used as a normalization control in the reactions (FIG. 13A). Emission at 517 nm due to catalysis by ERK-HH increased as the ERK2 concentration increased (FIG. 13B), while the activity of the constitutive ribozyme was unaffected by the presence of ERK2 as judged by emission at 568 nm (signal varied less than 3.2% in all measurements). The ratio of fluorescein emission to Cy3 emission provides a normalized index of ERK-HH activation (right ordinate, FIG. 13B); this profile correlated well with that observed in reactions employing radiolabeled substrate RNA and gel electrophoresis to detect ERK-HH activation (FIG. 10). These results show that nucleic acid sensor molecules can be used to quantitatively detect a target protein in a simple solution phase assay.

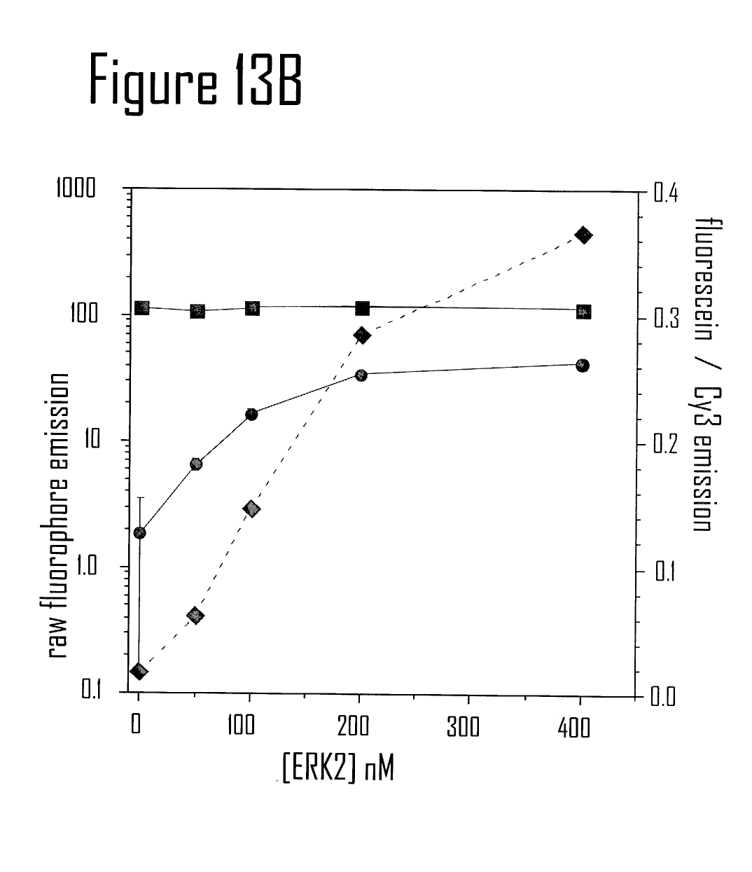

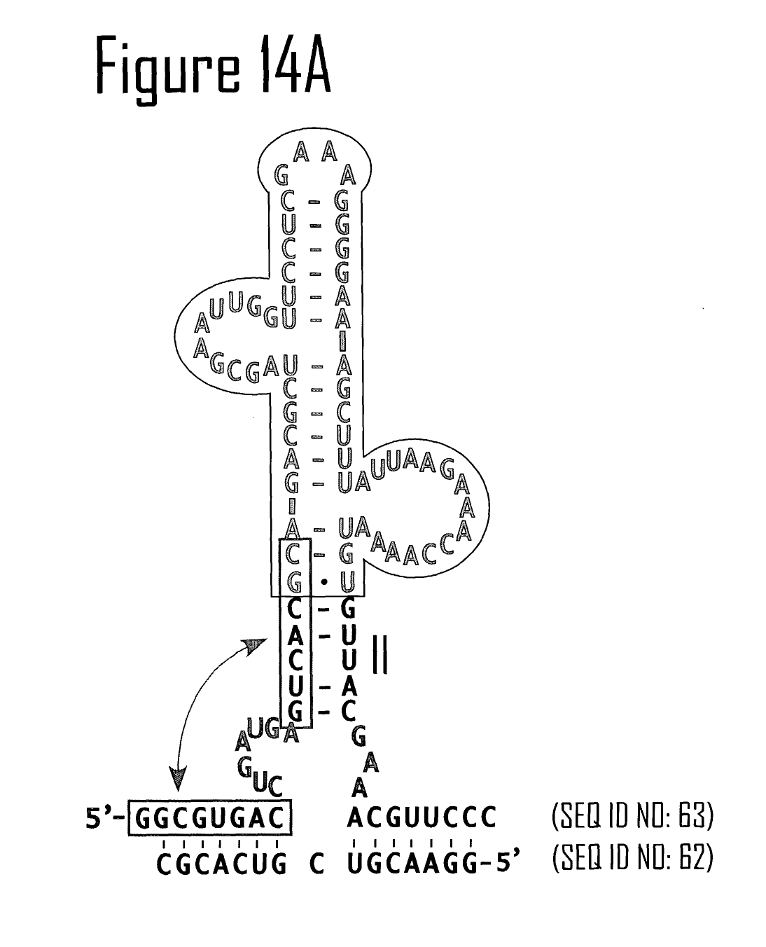

To test the generality of the design principles used to construct ERK-HH, a second protein-activated nucleic acid sensor molecule was constructed (ppERK-HH, FIG. 14A). In ppERK-HH, the high affinity ligand specific for unphosphorylated ERK2 was replaced with a high affinity RNA ligand specific for phosphorylated and activated ERK2 (Seiwert et al., 2001, Chem. Biol., 7, 833). Otherwise, ERK-HH and ppERK-HH are identical. The rate of substrate RNA (reporter molecule) cleavage promoted by ppERK-HH in the absence of protein was comparable to the uncatalyzed rate of phosphodiester bond hydrolysis of RNA under similar conditions (5.2×10⁻⁷ min⁻¹ at pH 7.5 versus 1.9×10⁻⁶ min⁻¹ at pH 8.0, respectively). However, phosphorylated ERK2 stimulated the observed rate of cleavage by ppERK-HH by ˜230-fold (FIG. 14B). Importantly, unphosphorylated ERK2 failed to activate catalysis by ppERK-HH to a level any greater than that observed in the absence of protein (FIG. 14B). Phosphorylated forms of related MAPKs (e.g., p38α and JNK2) which do not bind to the RNA ligand in ppERK-HH also failed to activate catalysis by ppERK-HH. Thus, although further combinatorial selection or rational engineering of protein-activated nucleic acid sensor molecules may be required to enhance catalytic rates, the rational design principles introduced here were generally applicable to develop nucleic acid sensor molecules capable of monitoring protein post-translational modifications.

Allosteric ribozymes have been described that respond to a variety of compounds. Here, applicant demonstrates that nucleic acid sensor molecules have sufficient specificity to also monitor the phosphorylation state of a target protein. The particular example involving selective activation of ERK-HH and ppERK-HH by opposite phosphorylation states of ERK2 (FIGS. 39B and 42B) is noteworthy because high resolution structural studies indicate that fewer than 10% of the amino acids in ERK2 differ in relative position by more than 1.1 Å upon phosphorylation (Canagarajah et al., 1997, Cell, 90, 859). Such specificity is ultimately a manifestation of the robustness of RNA combinatorial procedures which, in contrast to the specificity displayed by antibodies, can be readily defined and controlled.

The mechanism of activation of ERK-HH and ppERK-HH by their target analytes differs from that proposed for previously reported allosteric ribozymes: namely, it relies on an alternate conformer to diminish nucleic acid sensor molecule activity in the absence of target protein (FIGS. 9A and 37D). The strategy introduced here represents a general method for the production of protein-activated nucleic acid sensor molecules (FIGS. 37 and 42). Since this approach involves the generation of an inactive conformer by the sequestration of a substrate nucleic acid binding element, it should be equally applicable to enzymatic nucleic acid ligases and to enzymatic nucleic acids that carry modifications that confer stability in biological fluids.

A unique advantage of nucleic acid sensor molecules as protein sensing reagents is that they directly couple molecular recognition to signal generation and therefore provide simple assays for quantitative protein detection. A nucleic acid sensor molecule assay can simply involve adding nucleic acid sensor molecule and reporter substrate to a solution containing the molecular target, incubating the mixture, and measuring the nucleic acid sensor molecule activity (FIG. 13). The ability of nucleic acid sensor molecules to function in parallel in complex mixtures (FIG. 12) indicates the feasibility of using several nucleic acid sensor molecules to simultaneously monitor multiple classes of protein analytes in solution (FIG. 13). Nucleic acid sensor molecules also function well on solid supports that are suitable for more global profiling of protein expression using high density arrays. Taken together with the ability to produce large numbers of different functional RNAs through automated combinatorial selection, protein-responsive nucleic acid sensor molecules therefore can represent valuable reagents to globally monitor post-translational modifications of proteins in an arrayed format. Such flexibility in assay formats forecasts valuable roles for protein-activated nucleic acid sensor molecules in biological research and molecular diagnostics.

### Example 13

Selection and Characterization of HCV-Halfzyme Nucleic Acid Sensor Molecule

This example summarizes the results of a study investigating the development and use of Halfzyme™ Technology for the sensitive detection of nucleic acids. Halfzymes in this example are a class of RNA-based enzymes that, in the presence of a nucleic acid targeted for detection, direct the ligation of two reporter substrate oligonucleotides through a catalytic reaction. These Halfzyme enzymatic nucleic acids can be multiple turnover enzymes such that a single targeted nucleic acid-activated Halfzyme enzymatic nucleic acid produces many ligation products. Thus, Halfzyme enzymatic nucleic acid Technology provides an amplification step for nucleic acid detection.

The study was directed at the detection of sequences present in the Hepatitis C Virus (HCV) genome. Sequences in the 5′ untranslated region (UTR) were chosen as the target nucleic acid sequence because of their high degree of sequence conservation among different HCV strains.

Activities performed in the course of this study included: 1) development of efficient Halfzymes activated by HCV sequences (HCV-Halfzymes) through a robust, in vitro combinatorial process referred to as Directed Molecular Evolution (DME), 2) biochemical characterization and optimization of HCV-Halfzymes that function as multiple turnover enzymes, 3) determination of the limit of detection (L.O.D.) afforded by HCV-Halfzymes in an unformatted assay.

Applicant performed L.O.D. determinations in an unformatted assay in which HCV-Halfzyme ligation products were directly visualized and quantified. The L.O.D. assay utilized partially labeled substrate (therefore, less than 1.5% of products could be detected) and an instrument to quantify ligation products.

The L.O.D. of HCV sequence detection in this unformatted assay was 6000 molecules when a positive signal was judged as two standard deviations above noise (2 SD L.O.D.). This is a very conservative estimate of the unformatted assay L.O.D. since some experiments yielded detection of a little as 60 molecules and 2 SD L.O.D. values as low as 600 molecules. The kinetic characteristics of the HCV-Halfzymes developed in the study, however, indicate that when used in a properly formatted assay the true HCV-Halfzyme L.O.D will meet or exceed detection of 100 molecules.

Halfzyme Technology

Enzymatic nucleic acid molecules are nucleic acid-based enzymes that, like protein enzymes, accelerate biochemical reactions. Halfzymes, are a particular class of enzymatic nucleic acid molecule in which a portion of the nucleic acid sequence of the enzymatic nucleic acid molecule has been deleted and is supplied in trans as an effector nucleic acid. In an non-limiting example, Halfzymes are therefore devoid of catalytic activity in the absence of this effector sequence, but their activity can be induced through interaction with the trans-acting oligonucleotide effector. In the Halfzyme system an oligonucleotide target RNA acts as an effector or activator, inducing enzymatic nucleic acid molecule catalysis (FIG. 15). Consequently, Halfzyme catalytic activity acts as the readout for the presence of a particular target nucleic acid. Halfzymes function as multiple turnover catalysts, providing an intrinsic signal amplification step. Unlike PCR-based assays that require time consuming and difficult to automate thermal cycling, Halfzymes function in an isothermal manner. Moreover, the use of Halfzymes for nucleic acid detection does not require amplification of the target sequence, such that contamination problems associated with PCR-based assays are eliminated. Halfzyme enzymatic nucleic acid molecules are assembled from two or more separate nucleic acid molecules, preferably two or three separate nucleic acid molecules.

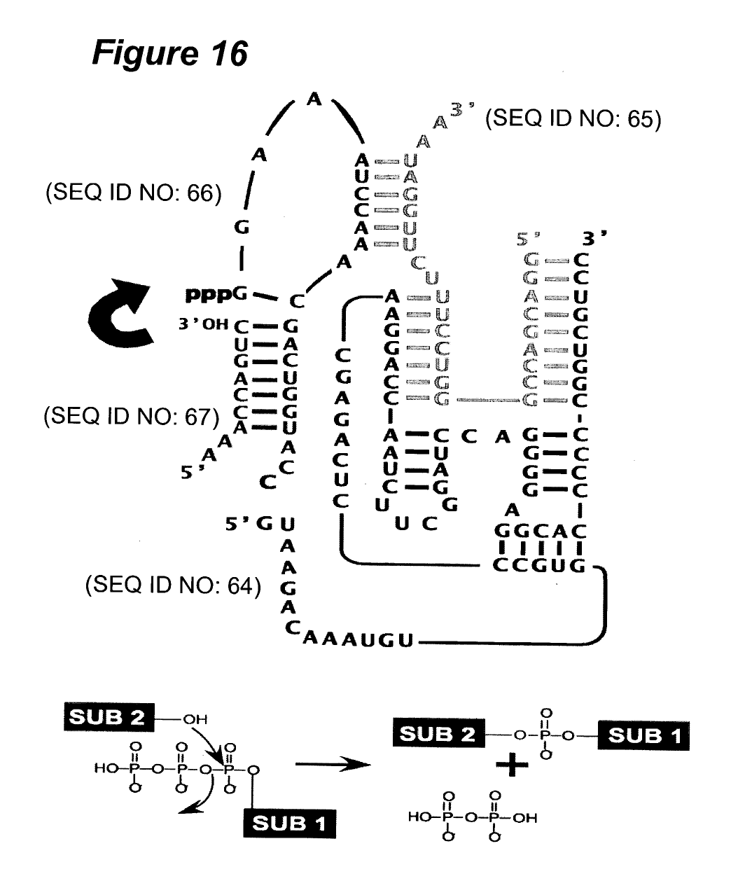

Halfzyme technology is based on, for example, an enzymatic nucleic acid that catalyzes ligation of separate molecule, for example a class I ligase enzymatic nucleic acid molecule (FIG. 16). This enzymatic nucleic acid molecule motif was chosen as the catalytic ‘platform’ for Halfzyme ligase development because it displays one of the fastest catalytic rates described for a enzymatic nucleic acid molecule (up to 300 turnovers per minute, and has been extensively characterized. In contrast to conventional hybridization-based nucleic acid detection strategies that require stretches of twenty or more highly conserved nucleotides, Halfzyme technology is designed so that target detection requires recognition of no more than nine contiguous nucleotides of conserved sequence (see for example FIG. 16).

The Halfzymes in this example covalently ligate two substrate (reporter) nucleic acids (FIG. 16). In the nomenclature used by applicant, the 5′ substrate RNA that supplies the nucleophilic hydroxyl in the ligation reaction is referred to as substrate 2. The 3′ substrate RNA that carries the 5′-triphosphate and hence the pyrophosphate leaving group is referred to as substrate 1. Each of these two substrates can be made by solid-phase oligonucleotide synthesis. Therefore, each substrate RNA can be independently and site specifically labeled with one or several haptens or capture reagents. Consequently, ligated products that are amplified from Halfzyme reactions can be detected in a number of different assay formats.

Hepatitis C Virus (HCV) Target Site

The use of Halfzyme technology as a method for the sensitive detection of viral sequences was tested. Halfzyme reagents were developed that are RNA sequences present in the Hepatitis C Virus (HCV) genome as effector molecules. Such Halfzymes are referred to as HCV-activated Halfzymes or HCV-Halfzymes.

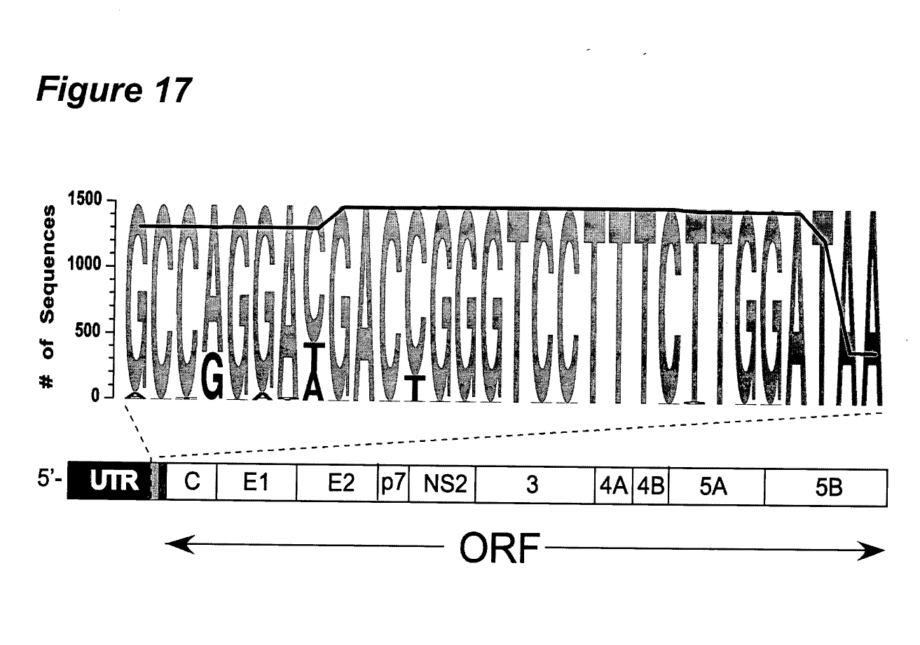

Consistent with nucleic acid testing strategies, it is preferable to select for detection of a viral nucleic acid sequence that is highly conserved among different viral strains. Hepatitis C Virus (HCV) is a positive strand RNA virus of approximately nine kilobases (9 kB) that contains one large open reading frame (ORF) encoding both structural and nonstructural proteins and a large 5′ untranslated region (5′-UTR), that acts as a functional RNA to direct cap-independent translation within cells. While the ORF is highly variable in nucleotide sequence, the 5′-UTR is conserved to a much greater degree. Thus, the 5′-UTR represents an attractive target for nucleic acid-based detection strategies. Within the 5′-UTR, the most highly conserved feature is a structural element referred to as stem-loop IIIB. A multiple sequence alignment of the approximately 1500 GENBANK entries of HCV sequences containing stem-loop IIIB indicates that the 3′ half of this sequence is almost universally conserved among HCV isolates while the 5′ portion varies only slightly (FIG. 17). Consequently, the sequence of stem-loop IIIB was chosen as the non-limiting example of a HCV-Halfzyme effector oligonucleotide (positions 175 to 205 by conventional numbering).

Directed Molecular Evolution (DME)

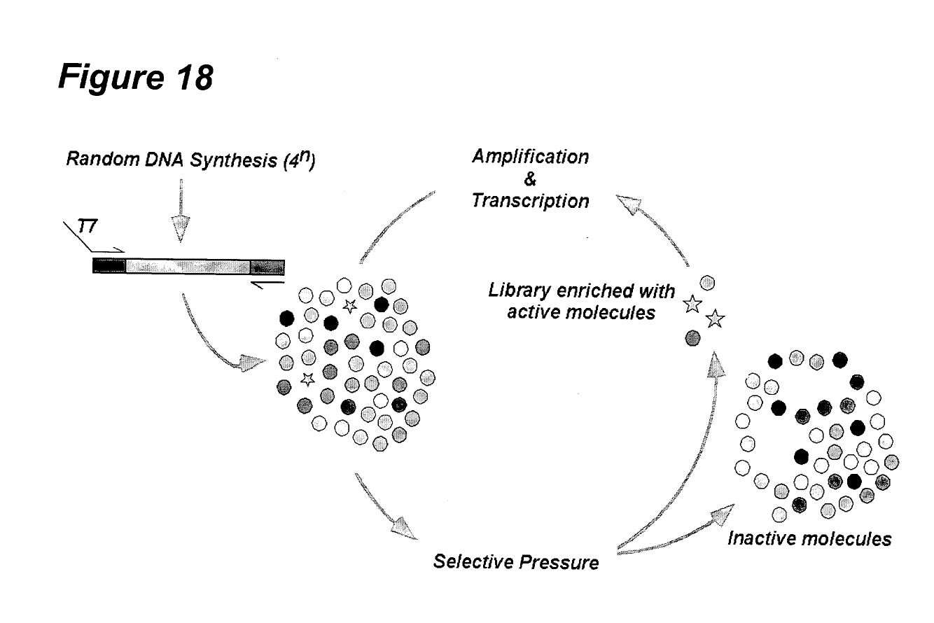

Nucleic acids possess the unique property that genotype (replicatable information) and phenotype (molecular function) reside in one molecule. Thus, molecules with specific functions can be directly replicated and amplified by RT-PCR. Directed Molecular Evolution, or DME, is a process for the combinatorial selection of nucleic acids in which a large collection of random sequence variants of a functional RNA are produced and subjected to selective pressure that allows rare sequence variants with enhanced performance characteristics to be the separated from the bulk of the sequence variants with less robust performance (FIG. 18). Formally, DME is similar to Darwinian evolution in that it entails ‘survival of the fittest’: many individuals (sequences) displaying different characteristics are forced to ‘compete’ by subjecting them to a selective pressure. Those enzymatic nucleic acid molecule sequences with the best performance are allowed to reproduce (DME reproduction is through RT-PCR rather than organismal reproduction as in true evolution). After amplification, the library of sequences that is enriched for functional molecules is used in subsequent cycles of DME in which the stringency of the selective pressure is progressively increased. When the properties of the library of sequences as a whole display the desired functional characteristics, individual members are cloned through bacterial transformation and individually characterized. In this way, enzymatic nucleic acid molecules with properties substantially better than the starting sequence can be identified.

Applicant has used DME to develop catalytically efficient Halfzymes that are activated by a predetermined effector nucleic acid: sequences of stem-loop IIIB of the HCV 5′-UTR. This work serves as a model for the production of Halfzymes that are activated by other target nucleic acids. All work performed in this example utilized the class I ligase enzymatic nucleic acid molecule as the catalytic ‘platform’ for the production of Halfzyme ligase catalysts. A person skilled in the art will recognize that other enzymatic nucleic acid molecules can similarly be utilized for the production of Halfzyme or other multicomponent nucleic acid constructs using the teaching of this application, including nucleic acid sensor constructs capable of catalyzing different chemical reactions.

Applicant first defined the Limit of Detection (L.O.D.) of a single turnover version of an HCV-activated Halfzyme. This HCV-Halfzyme promotes the ligation of a substrate RNA oligonucleotide to its own 5′ terminus. Thus, it is able to perform only a single catalytic cycle; it does not provide amplification through multiple turnover catalysis.

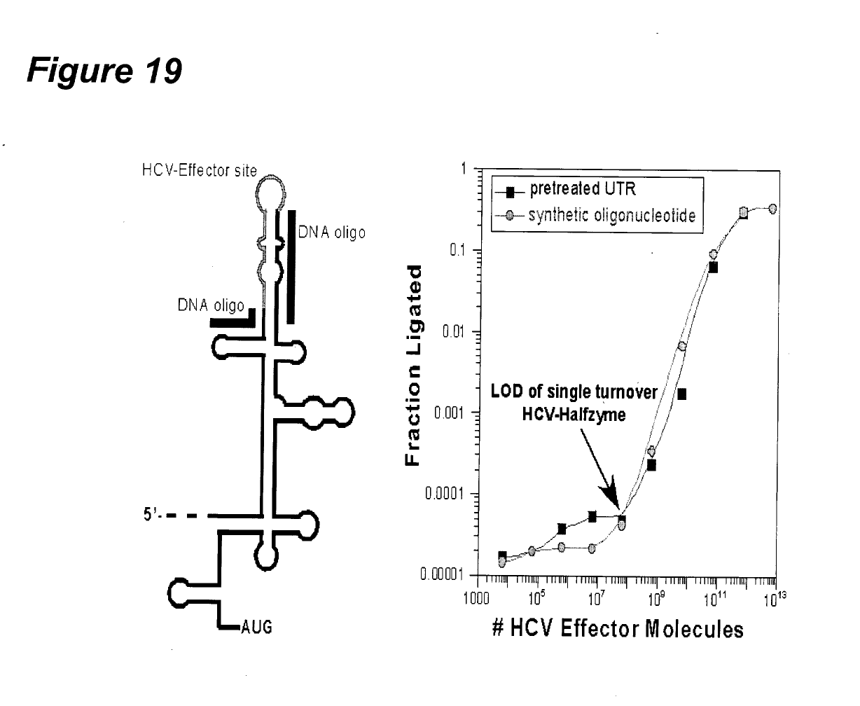

The sequence of the HCV-Halfzyme used in these initial studies contained sequence changes to accommodate the sequence of the HCV target oligonucleotide (FIG. 19). The L.O.D. was established by determination of HCV-Halfzyme activity as a function of the copy number of the HCV effector sequence. Using a synthetic oligoribonucleotide representing stem-loop IIIB of the HCV 5′ UTR target site, the limit of detection of this system was 6×10⁷ molecules. Importantly, Halfzymes that were activated by the complete HCV 5′-UTR displayed the same L.O.D. as the synthetic oligonucleotide if the 5′-UTR was first pre-treated with RNase H and specific DNA oligonucleotides to liberate the HCV-Halfzyme effector nucleic acid (FIG. 19). Since the 5′-UTR folds into an independent structural domain within the intact the HCV genome, the ability to activation of the HCV-Halfzyme with an effector nucleic acid derived from the 5′-UTR indicates that it can be activated with sequences derived by the intact HCV genome when isolated from clinical samples.

The above work demonstrated the efficacy of using Halfzyme technology to detect viral nucleic acid sequences. However, the L.O.D. of this single turnover HCV-Halfzyme may not be sufficient for use in a number of blood screening applications. Multiple turnover versions of such an HCV-Halfzyme, therefore, were needed to carry out multiple catalytic cycles for signal amplification. However, the catalytic rate of the HCV-Halfzyme used in the above studies was extremely low (k_{obs}=˜5×10⁻⁴ min⁻¹). Consequently, the production of an HCV-Halfzyme with better performance characteristics was required to decrease the L.O.D. to levels appropriate for viral screening. To develop a more efficient HCV-Halfzyme, Directed Molecular Evolution (DME) was performed to identify sequence variants of the HCV-Halfzyme described above with the desired performance characteristics.

Initial HCV-Halfzyme Development in DME-1

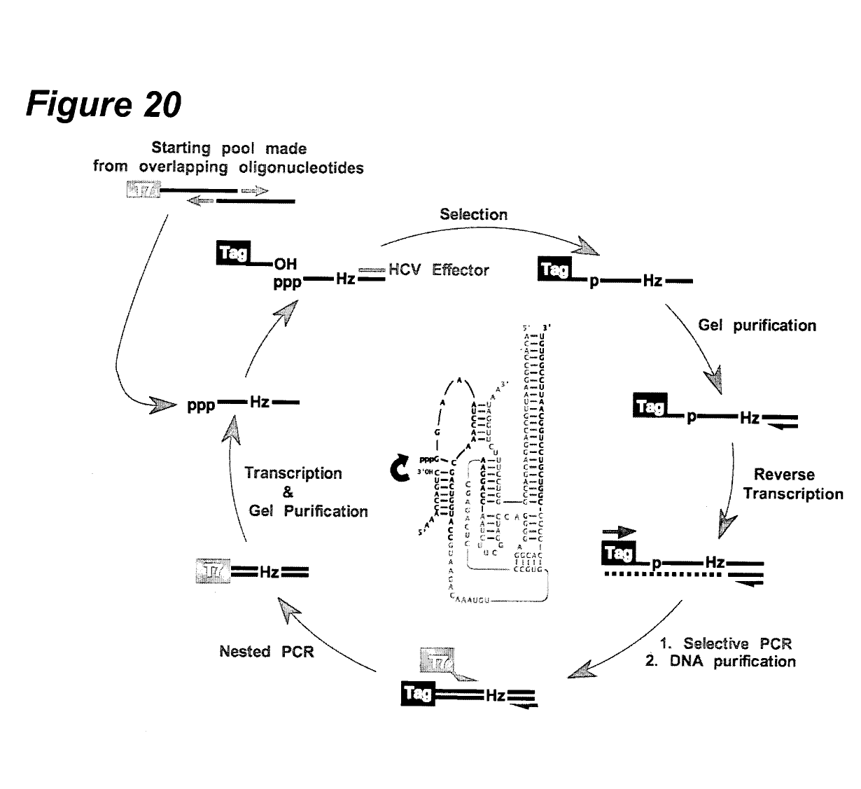

The HCV-Halfzyme sequence library used for DME-1 was produced through ‘doped’ solid phase oligonucleotide synthesis of DNA by standard procedures. A total of 62 nucleotide positions in regions of the HCV-Halfzyme not directly involved in interaction with substrate RNA or hybridization to the HCV effector were mutagenized such that each consisted of non-parental sequence 30% of the time (FIG. 20). As a result, each of the ˜10¹⁰ HCV-Halfzyme sequence variants carried ˜18 changes relative to the parental HCV-Halfzyme. In this HCV-Halfzyme library, HCV effector hybridized to stretches of 6, 7, and 24 nucleotides.

Each cycle of DME-1 involved Halfzyme transcription, incubation with HCV effector oligonucleotide and substrate 2, fractionation and collection of substrate 2/HCV-Halfzyme ligated product from non-reacted HCV-Halfzyme by gel electrophoresis, amplification of substrate 2/HCV-Halfzyme ligated product by RT-PCR, and a second PCR to regenerate the transcriptional promoter and appropriate 5′ end of the Halfzyme pool (FIG. 20). Each cycle of this selection required ˜5 days. In successive cycles of DME-1 the selective pressure for faster catalytic rates was applied by decreasing the time allowed for ligation of substrate 2 to the HCV-Halfzyme sequence library. Initially, this reaction time was 16 h, but by the seventh and final round the incubation time was reduced to 15 seconds.

At each cycle of DME, HCV-Halfzymes with progressively faster catalytic rates were enriched (data not shown). DME-1 was terminated after seven cycles of DME-1, at which point the HCV-Halfzyme library displayed a single turnover rate of 0.03 min⁻¹. This rate is similar to that obtained in the initial selection of this ligase enzymatic nucleic acid molecule. DNA representing individual sequences contained in this enriched HCV-Halfzyme library were ligated into bacterial plasmids and isolated by transformation into E. coli. Individual HCV-Halfzymes were then characterized as described below.

Characterization of HCV-Halfzymes Developed in DME-1

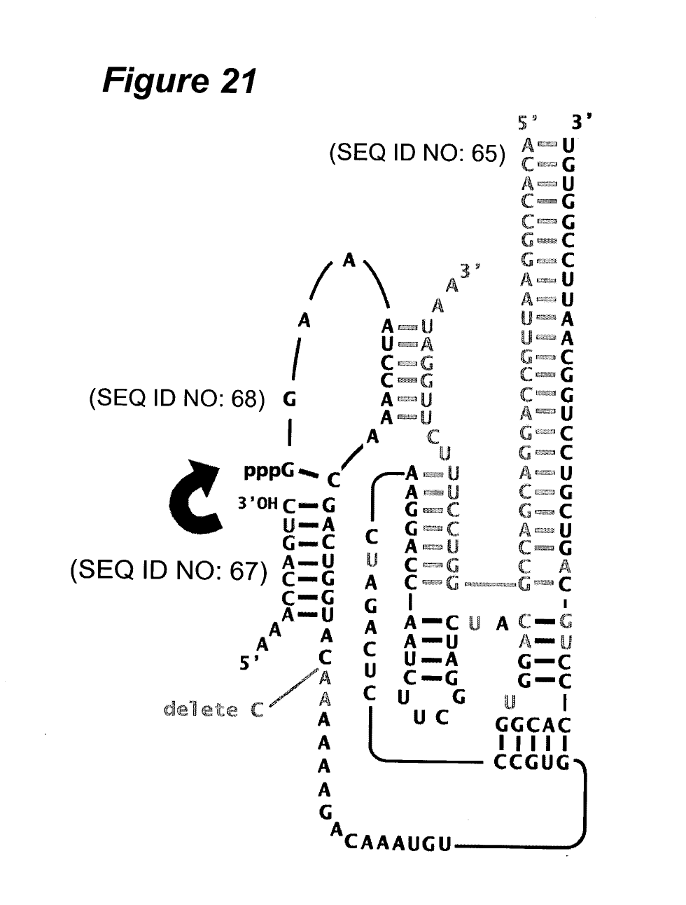

The kinetic properties (Table II) and sequences of clones representing thirty-three HCV-Halfzymes developed in DME-1 were characterized. Significantly, several sequence variants display an observed rate of ˜1.5 to 2 min⁻¹. These HCV-Halfzymes comprised one sequence family. The optimal HCV-Halfzyme from DME-1, clone 8/7, contains 10 nucleotide changes relative to the input sequence and one nucleotide deletion (FIG. 21). These changes are responsible for increasing its activity in auto-ligation (single turnover) reactions by ˜400-fold. The catalytic rate of 8/7 HCV-Halfzyme was characterized in terms of its dependence on pH and Mg²⁺. The assembly of 8/7 HCV-Halfzyme into active complexes and its ability to function as a multiple turnover enzymatic nucleic acid molecule were also investigated.

TABLE II

| Clone # | Rate (min⁻¹) |
|---|---|
| 8/7 | 2.100 |
| 8/24 | 1.650 |
| 8/26 | 1.610 |
| 8/3 | 1.340 |
| 8/6 | 1.260 |
| 8/5 | 1.190 |
| 8/8 | 1.060 |
| 8/16 | 0.994 |
| 8/15 | 0.850 |
| 8/10 | 0.810 |
| 8/21 | 0.730 |
| 8/4 | 0.670 |
| 8/19 | 0.630 |
| 8/20 | 0.600 |
| 8/27 | 0.530 |
| 8/13 | 0.500 |
| 8/11 | 0.400 |
| 8/25 | 0.380 |
| 8/12 | 0.330 |
| 8/14 | 0.320 |
| 8/1 | 0.310 |
| 8/23 | 0.300 |
| 8/18 | 0.290 |
| 8/2 | 0.230 |
| 8/32 | 0.030 |
| 8/29 | 0.023 |
| 8/22 | 0.016 |
| 8/17 | 0.010 |
| 8/28 | 0.004 |
| 8/30 | 0.004 |
| 8/33 | 0.002 |
| 8/9 | 0.001 |

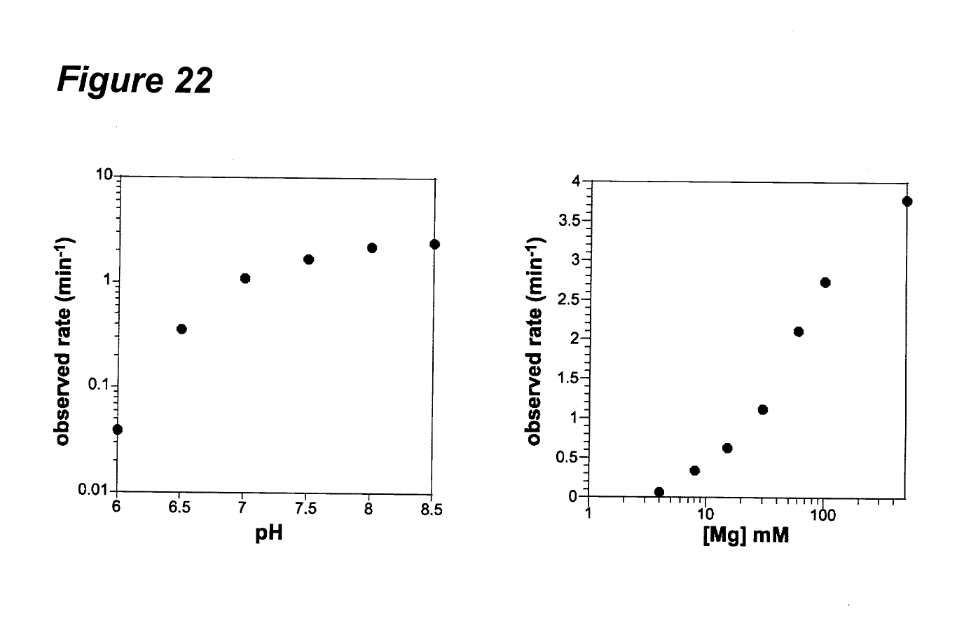

A log-linear relationship between pH and observed rate of 8/7 HCV-Halfzyme would mean that the chemical step is limiting the observed rate since the chemical step of ligation promoted by the class I ligase enzymatic nucleic acid molecule is dependent on the concentration of hydroxide ion in solution. Increasing pH, therefore, could be used to increase its catalytic activity. The observed single turnover rate of clone 8/7 HCV-Halfzyme displayed a log-linear relationship with pH until pH 7.0, at which point the rates reached a plateau at ˜2 min⁻¹ (FIG. 22). Increasing pH further did not result in an increase in observed rate. Such a plateau indicates that the observed rate of this HCV-Halfzyme is limited by a conformational rearrangement proceeding at ˜2 min⁻¹.

The dependence of the observed rate on Mg²⁺ was examined since Mg²⁺ is believed to affect the folding of enzymatic nucleic acid molecules, and therefore could affect the rate-limiting conformational rearrangement of the 8/7 HCV-Halfzyme that is evidenced in its pH profile. Indeed, the catalytic rate of the 8/7 HCV-Halfzyme increased as a function of Mg²⁺ at all concentrations tested, indicating that the folding of 8/7 HCV-Halfzyme was incomplete at even the highest Mg²⁺ concentration tested (500 mM, FIG. 22). Thus, the optimal HCV-Halfzyme obtained from DME-1 is limited in rate by its incomplete formation of an active three-dimensional structure in a way that could not be compensated by high concentrations of Mg²⁺.

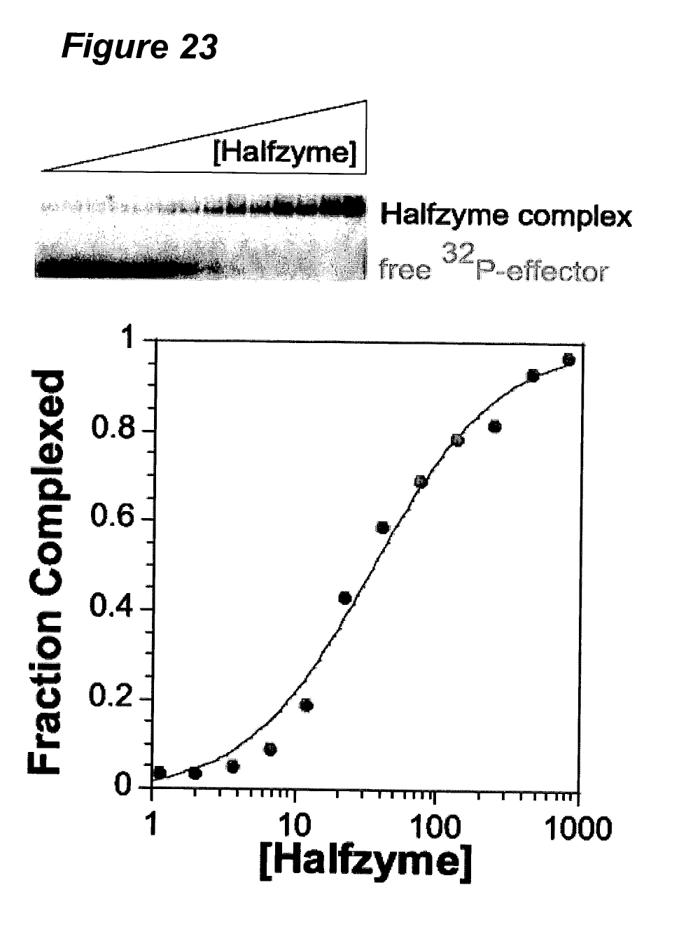

To examine whether the gross assembly of the 8/7 HCV-Halfzyme was the cause of its folding deficiency, a native gel electrophoresis assay was developed to monitor RNA-RNA interactions (FIG. 23). This assay was used to determine the affinity of all relevant RNA-RNA interactions required for assembly and function of active HCV-Halfzyme complexes. These include the affinity of substrate 2 for the HCV-Halfzyme and the affinity of the HCV-Halfzyme for the HCV effector oligonucleotide. These data showed that all relevant assembly events proceeded to 100% completion at the concentrations of substrate 2 RNA, HCV-Halfzyme and HCV effector oligonucleotide used in assays (data not shown). Thus, the deficiency in folding displayed by the 8/7 HCV-Halfzyme could not be accounted for by inefficient or incomplete assembly events.

Conversion of the single turnover 8/7 HCV-Halfzyme obtained from DME-1 to a multiple turnover version involves dissection into two parts: a 5′ portion becomes a trans acting substrate (substrate 1), and a 3′ portion that becomes the multiple turnover HCV-Halfzyme. Substrate 1 is ligated to the same substrate 2 RNA that is used in single turnover reactions. By dissecting the HCV-Halfzyme at various internucleotide positions several multiple turnover configurations of the clone 8/7 HCV-Halfzyme were generated (Table III). Each directs the ligation of distinct substrate 1 RNA oligonucleotide to the substrate 2 oligonucleotide.

Multiple turnover configurations analogous to that described for the parental class I ligase enzymatic nucleic acid molecule had low activity (multiple turnover rates less than 10⁻⁵ min⁻¹, Table III). However, a configuration that utilizes pppGGA as substrate 1 showed a turnover rate of 0.004 min⁻¹. This value is approximately 200-fold lower than the observed rate displayed by the single turnover clone 8/7 HCV-Halfzyme. This particular configuration for a multiple turnover HCV-Halfzyme is attractive because the uncatalyzed rate of substrate RNA ligation is minimized since the reactive groups (5′triphosphate and 3′ hydroxyl of the two substrate RNAs) are not held in close proximity as they are in the unimolecular substrate RNA-substrate RNA complex used in other configurations.

TABLE III

| | | | |
|---|---|---|---|
| | | Rate | |
| Configuration | Substrate 1 Sequence (5′-3′) | (min-1) | SEQ ID NO |

| | | | |
|---|---|---|---|
| 1 | ppp-GGA | 0.004 | 76 |
| 2 | PPP-GGAAAUCCAAACGACUGGUAC | <10-5 | 77 |
| 3 | PPP-GGAAAUCCAAACGACUGGUACAAAA | <10-5 | 78 |
| 4 | ppp- | <10-5 | 79 |
| | GGAAAUCCAAACGACUGGUACAAAAAAGACAAU | | |
| 5 | ppp- | <10-5 | 80 |
| | GGAAAUCCAAACGACUGGUACAAAAAAGACAAAU | | |
| | GUGUGCCCUCA | | |
| 7 | ppp-GGAAAUCCAAACGACUG | <10-5 | 81 |

Secondary HCV-Halfzyme Development in DME-2

A secondary DME (DME-2) was initiated to optimize folding and further increase catalytic activity of the Halfzyme constructs. DME-2 was carried out to optimize folding and further increase activity of 8/7 HCV-Halfzyme obtained from DME-1. Since the activity of 8/7 HCV-Halfzyme was believed to be limited by the binding of Mg²⁺, in DME-2 HCV-Halfzymes were demanded to display increased catalytic function at reduced concentrations of Mg²⁺.

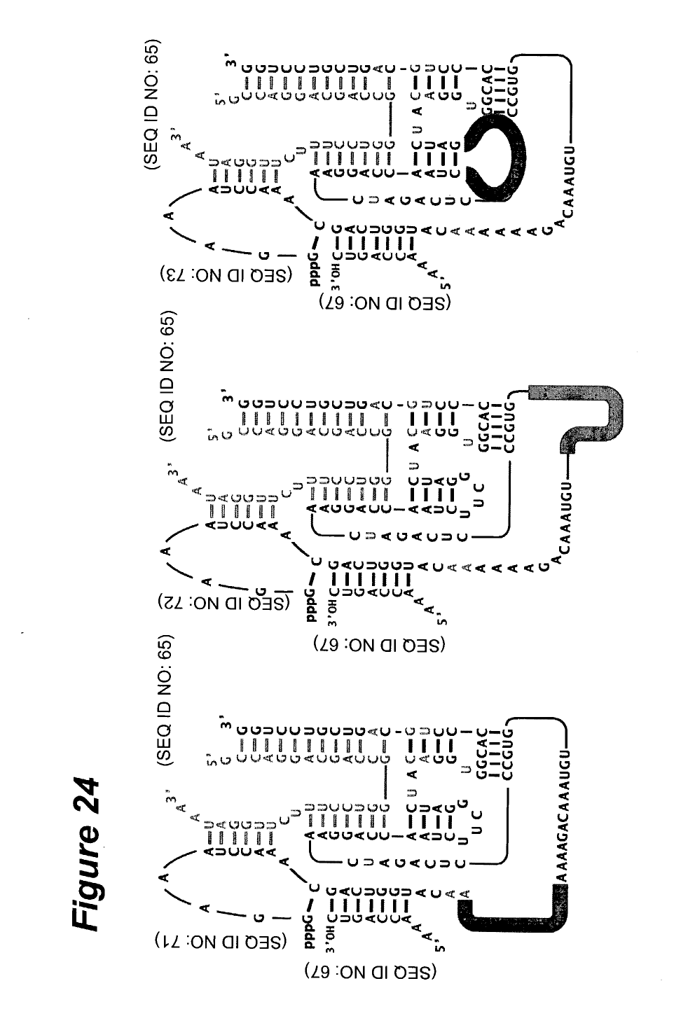

In DME-2 a new library of HCV-Halfzyme sequence variants was produced based on the clone 8/7 HCV-Halfzyme obtained from DME-1. In DME-2, rather than simply re-randomizing the positions already present in the clone 8/7 HCV-Halfzyme, additional regions of random sequence were added to the existing clone 8/7 HCV-Halfzyme (FIG. 24). Three different HCV-Halfzyme libraries were produced for three DME-2 processes that were performed in parallel. In two of these libraries, 30 nucleotides of random sequence were inserted. In the third HCV-Halfzyme library, 26 random nucleotides replaced a four nucleotide loop within the clone 8/7 HCV-Halfzyme. Each of these three libraries consisted of ˜1×10¹⁵ HCV-Halfzyme sequence variants. Importantly, all three of the HCV-Halfzyme sequence libraries used in DME-2 was 3′-truncated relative to the 8/7 HCV-Halfzyme obtained from DME-1. Thus, HCV-Halfzymes produced in DME-2 can contiguously hybridize to nine nucleotides of HCV sequence.

DME-2 was carried out similar to DME-1, except that: 1) the concentration of Mg²⁺ was progressively decreased in successive rounds, and 2) each of the three libraries was subjected separately to DME-2. After each cycle of DME-2, Halfzymes with faster catalytic rates were obtained even as the concentration of Mg²⁺ was reduced (data not shown). To obtain the best performing HCV-Halfzymes from these three sequence libraries, all three libraries were mixed together and subject to the final cycle of DME-2 in which they were made to compete against one another. DME-2 was terminated after this cycle at which point the pooled HCV-Halfzyme library displayed a single turnover rate of 0.02 min⁻¹ at 1 mM Mg²⁺.

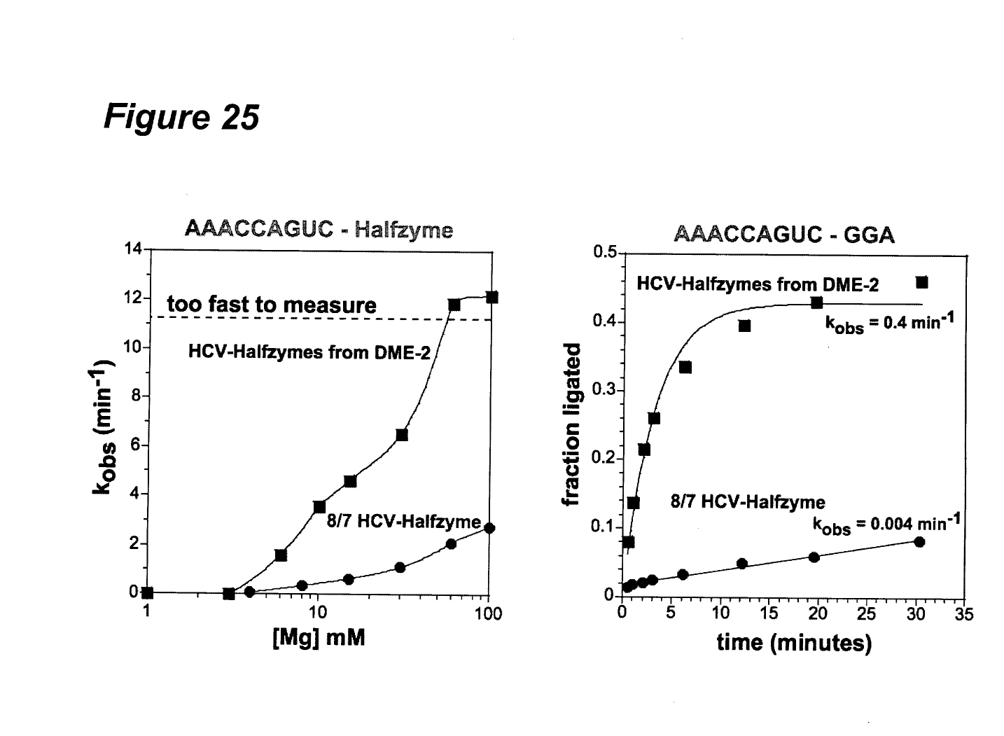

Single turnover ligation rates of the enriched library of HCV-Halfzyme sequences obtained in DME-2 were determined as a function of Mg²⁺ concentration and compared to the same data set obtained for the 8/7 HCV-Halfzyme obtained from DME-1. At all Mg²⁺ concentrations tested, the rates of ligation promoted by the HCV-Halfzyme library obtained from DME-2 were dramatically higher than those displayed by the 8/7 HCV-Halfzyme from DME-1 (FIG. 25). Indeed, observed rates of the HCV-Halfzyme library obtained from DME-2 were too fast to measure at Mg²⁺ concentrations above 30 mM (>12 min⁻¹, FIG. 25.

To directly assess ability of the HCV-Halfzymes from DME-2 to function in multiple turnover format, the rate of ligation of substrate 2 to pppGGA was tested using an appropriately 5′-truncated HCV-Halfzyme library. An improvement of at least 200-fold in this reaction was observed relative to the activity of 8/7 HCV-Halfzyme (FIG. 25), suggesting that this sequence library contained HCV-Halfzymes that could efficiently function in multiple turnover format. Thus, characterization of individual HCV-Halfzymes from this library was undertaken. As in DME-1, DNA representing individual sequences obtained from DME-2 were ligated into bacterial plasmids, isolated by transformation into E. coli and individual HCV-Halfzymes characterized as described below.

Characterization of HCV-Halfzymes from DME-2

Eighty clones from the final DME-2 library were transcribed and their single turnover rates characterized under sub-optimal conditions (so that their rates would be slowed enough to quantify relative to the rate displayed by the complete library of HCV-Halfzymes). Twenty-one of these clones displayed a rate greater than the final selected pool. Nineteen of these twenty-one clones were sequenced and shown to comprise one family of related sequences. The seven displaying the best performance in this initial kinetic screen were characterized in more detail at pH 7.5, 3 mM Mg²⁺ (Table IV). While the catalytic rates of these seven clones were rather tightly clustered, clones 21 and 38 displayed the fastest rates and proceeded to 86% and 74% completion, respectively, under these conditions. Note that the plateau value in single turnover conditions can be used to judge the fraction of the HCV-Halfzyme that is active for ligation. These criteria were used to establish clone 38 and clone 21 HCV-Halfzymes as our lead reagents. Both HCV-Halfzymes display a rate of ligation that is equal to that reported for the class I constitutive ligase from which they were derived, even though the latter was measured at an even higher Mg²⁺ concentration (Table, IV). Under optimal conditions (pH 7.5, 60 mM Mg²⁺) both of these clones displayed single turnover rates that were >15 min⁻¹ (too fast to measure by manual pipetting methods, data not shown).

TABLE IV

| Clone # | k_{obs} (min⁻¹), Plateau |
|---|---|
| 38* | 4.1, 86% |
| 21* | 3.6, 74% |
| 26* | 3.3, 85% |
| 30* | 3.1, 85% |
| 17* | 3.0, 84% |
| 35* | 2.5, 88% |
| 24* | 2.1, 79% |
| constitutive | 3.9, 70% |
| ligase¹ | |

*pH 7.5, 3.0 mM Mg²⁺
¹pH 7.5, 10.0 mM Mg²⁺

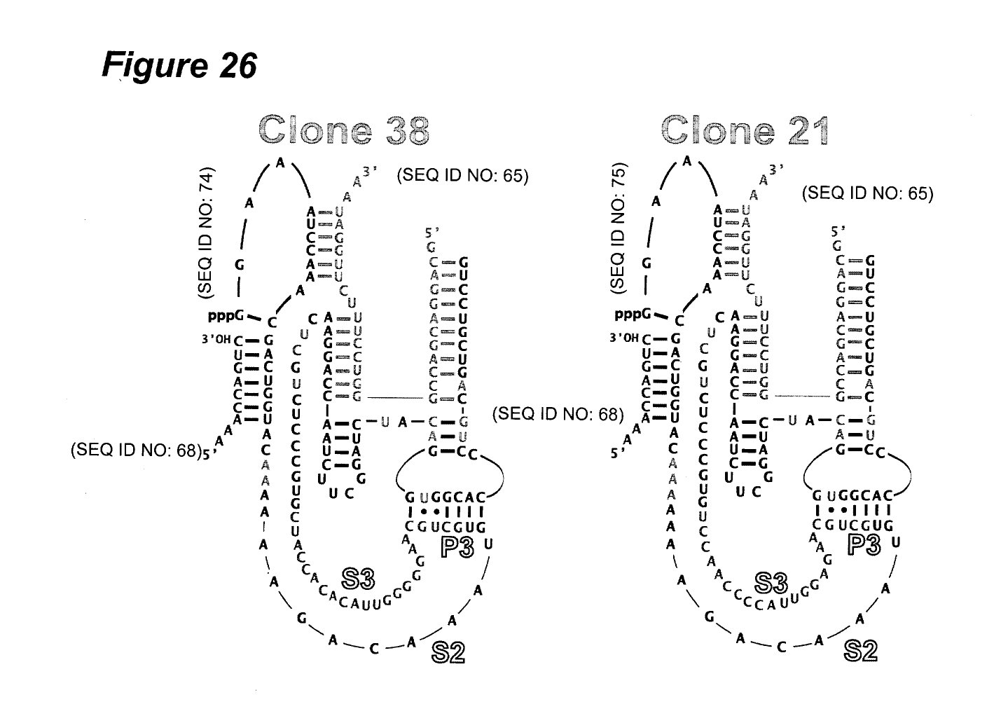

Clones 21 and 38 are closely related in sequence and predicted secondary structure (FIG. 26). In both HCV-Halfzymes, the sequence selected from the random sequence library served to shift, or slide, the base paired region (referred to as P3) 3′ of its original location, dramatically changing the orientation of the highly conserved unpaired nucleotides (designated S2 and S3) thought to comprise the catalytic core of the class I enzymatic nucleic acid molecule motif.

Multiple Turnover HCV-Halfzymes

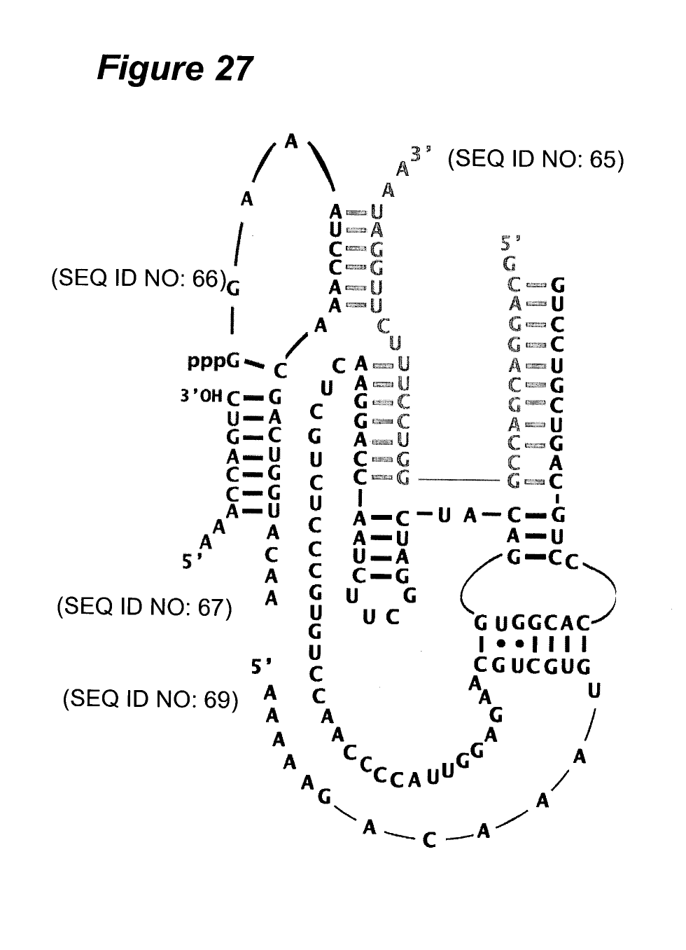

Several multiple turnover configurations of clone 38 and clone 21 HCV-Halfzymes were developed by 5′ truncation at various positions. However, work focused on two (referred to as configuration 1 and configuration 3, FIG. 27). Each multiple turnover configuration requires an HCV-Halfzyme that is uniquely 5′- truncated and a substrate 1 of a different length. The properties of each of these configurations are quite distinct and each is described separately below.

Characterization of Configuration 3

This configuration of multiple turnover HCV-Halfzyme was produced by 5′-truncating 23 nucleotides from the 5′ end of the single turnover version of clone 21 HCV-Halfzyme. This same sequence functions in trans as substrate 1 (FIG. 27). In configuration 3, the two substrates base pair with one another to form a unimolecular complex independent of the HCV-Halfzyme. This substrate RNA complex associates with effector-bound HCV-Halfzyme through Watson-Crick base interaction with effector nucleic acid sequence. Thus, the substrate RNA complex will not associate with HCV-Halfzyme in the absence of the HCV effector nucleic acid.

Configuration 3 is very similar to the multiple turnover version of the class I ligase enzymatic nucleic acid molecule. To date, the maximal turnover rate of clone 38 or clone 21 HCV-Halfzyme in this configuration is 0.75 min⁻¹ (assayed at pH 7.5, 60 mM Mg²⁺ and 12.5 uM substrate RNAs). This rate is approximately 20-fold lower than the rate of the single turnover reaction promoted by clone 21 HCV-Halfzyme under identical conditions (>15 min⁻¹). Additional optimizations are carried so that the multiple turnover rate matches the single turnover rate, which will proportionally decrease the L.O.D. afforded by this configuration of clone 21 HCV-Halfzyme.

The native gel electrophoresis system previously described (see Characterization of HCV-Halfzymes obtained through DME-1 and Materials and Methods) was used to investigate the substrate RNA concentrations required for efficient association, and the concentration of HCV-Halfzyme required for complete capture of the HCV effector oligonucleotide. This analysis showed that substrate 1 and substrate 2 have an affinity for one another of 5 nM (data not shown). The affinity of the product of ligation (produced synthetically) for HCV-Halfzyme bound to HCV effector was 1.3 uM (data not shown). As expected, the simulated product did not show any interaction with HCV-Halfzyme in the absence of effector. The affinity of the HCV-Halfzyme for the HCV-effector nucleic acid was 48 nM. Complete capture of effector by HCV-Halfzyme occurred at 500 nM and set the concentration of HCV-Halfzyme used in L.O.D. determinations. Complete saturation of substrate RNA complex to the effector bound HCV-Halfzyme was achieved at concentrations of ˜13 uM.

Optimization of Configuration 3 for 32P L.O.D. Determinations

Conditions were optimized for determining the L.O.D. of clone 21 HCV-Halfzyme in configuration 3. The assay for L.O.D. determination utilizes substrate RNA that is partially labeled with ³²P, gel electrophoresis to resolve ligated product from substrate, and phosphoimage analysis for the direct visualization and quantification of ligated product. Because the L.O.D. determinations were performed with partially labeled substrate RNA, maximization of detectable signal required substrate RNA concentrations that did not support fully catalytic activity of the HCV-Halfzyme. Thus, in the assay performed not every cycle of HCV-Halfzyme catalysis can be monitored in the L.O.D. determinations.

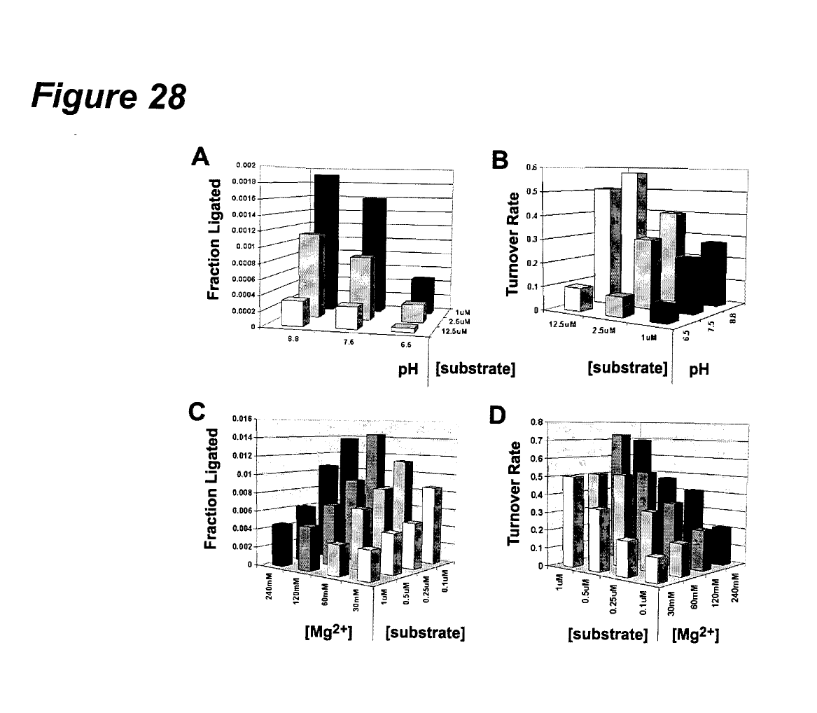

To optimize signal in the L.O.D. determinations using the ³²P assay, HCV-Halfzyme signal in the presence of 1×10⁷ effector molecules was investigated as a function of pH, substrate RNA concentration and Mg²⁺ concentration (FIG. 28).

Examination of the dependence of signal and turnover rate on pH and substrate concentration shows that clone 21 HCV-Halfzyme turnover rate and detectable signal increases 5-fold from pH 6.5 to 7.5 (5-fold increase), but begins to plateau at higher pH values (FIGS. 28A,B). As expected, signal increased at every pH as substrate RNA was decreased (FIG. 28A). However, turnover rate decreased (FIG. 28B). To more closely examine the effect of lowering substrate RNA concentration on signal and turnover rate a second optimization was conducted at lower substrate RNA concentrations (FIGS. 28C,D). This optimization was performed as a matrix with varying Mg²⁺ concentration since the affinity of the substrate RNA complex for clone 21 HCV-Halfzyme could be influenced by Mg²⁺ concentration. As expected, these data showed that signal increased as the substrate RNA concentration decreased (FIGS. 28C,D). Maximal signal was obtained at 60 to 120 mM Mg²⁺. This analysis was used to establish the following conditions for L.O.D. determinations using partially radiolabeled substrate RNA: pH 7.5, 60 mM Mg²⁺. L.O.D. determinations were performed at several substrate RNA concentrations. Additional trials to more finely define the optimal conditions for L.O.D. determinations are carried out to fully optimize assays using the ³²P-based assay.

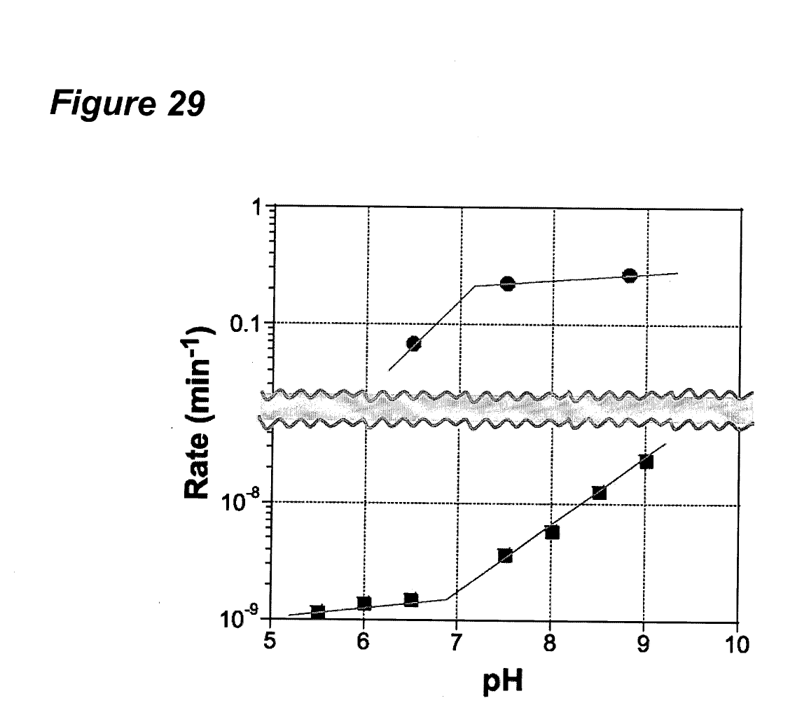

Since pH effects HCV-Halfzyme activity (and consequently L.O.D.), the dependence of the ligation rate on pH was analyzed in the presence and absence of HCV effector. The resultant curves show very different profiles (FIG. 29). The maximal rate difference between + effector and − effector ligation rates occurs between pH 7.5 and 6.5. The L.O.D. determinations reported below were carried out at pH 7.5; at this pH the rate differential between in the presence and absence of effector is 6.2×10⁷.

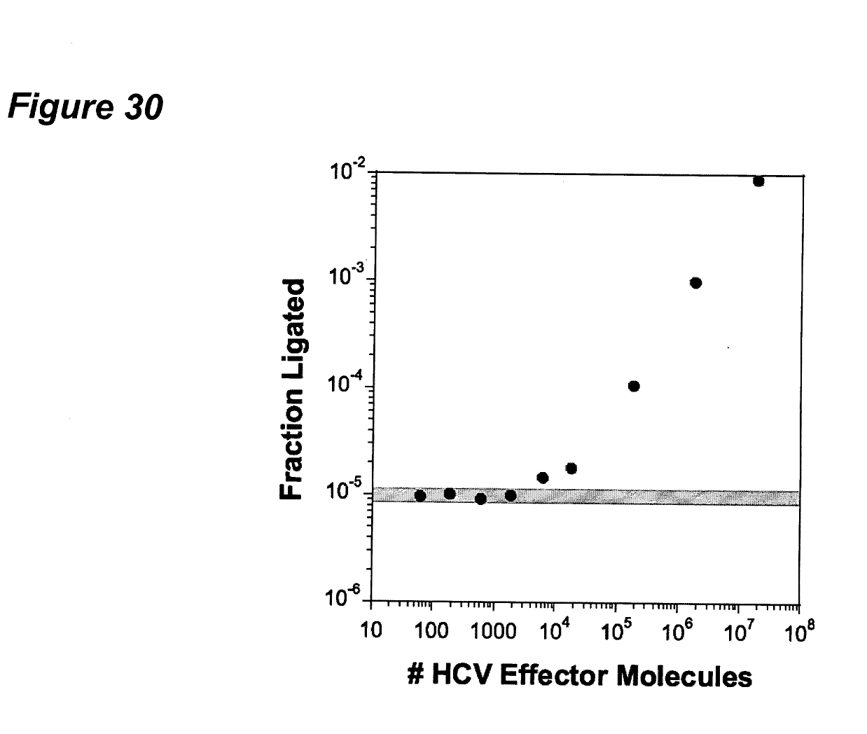

All L.O.D. determinations were carried out at n=5. Positive signal was judged as an average signal from the 5 trials that was greater than 2 standard deviations above the signal seen in the absence of HCV effector (2SD L.O.D.). Trials were conducted at pH 7.5, 60 mM Mg2+, 0.5 to 0.1 uM clone 21 HCV-Halfzyme and 0.5 uM substrates 1 and 2. Under these conditions, the average 2SD L.O.D. of the clone 21 HCV-Halfzyme in configuration 3 is 1,800 molecules (FIG. 30). In all of the HCV-Halfzyme L.O.D. determinations performed, fluctuations in system background, the level of uncatalyzed ligation of the substrate RNAs, and assay operator variability from experiment to experiment allowed the 2SD L.O.D. to range from 600 molecules to 18,000 molecules. Detectable signal above background was observed as low as 60 molecules. Coefficient of Variance (CV) values in L.O.D. determinations typically ranged from 10 to 20%.

Several characteristics of the assay performed reduce or limit assay sensitivity and increase CV. Due to the signal detection capabilities of the phosphoimager instrument used to quantify signal and the use of partially labeled substrate RNA, Halfzyme reactions were carried out for long incubation times (usually 18 hours or longer). During such long incubation times, a loss of Halfzyme activity is expected. Uncatalyzed, ‘background’ ligation due to the intrinsic chemical reactivity of the two substrates is constant during this incubation period. Consequently the ratio of signal (+effector Halfzyme catalysis) to noise (uncatalyzed background ligation) increases when incubation times are increased. Further, to assess the amount of ligated product RNA, it was separated from substrate RNA based on its mobility in gel electrophoresis. Due to the nature of gel electrophoresis, a small amount of the substrate RNA always “bleeds” into the position where the ligated product migrates. This “bleeding” creates a background of radioactivity and impacts our ability to visualize ligated product. Detection strategies that utilize completely labeled substrate RNA and that are more sensitive than phosphoimage analysis are likely to result in a decrease in the L.O.D. afforded by Halfzyme technology.

Configuration 1

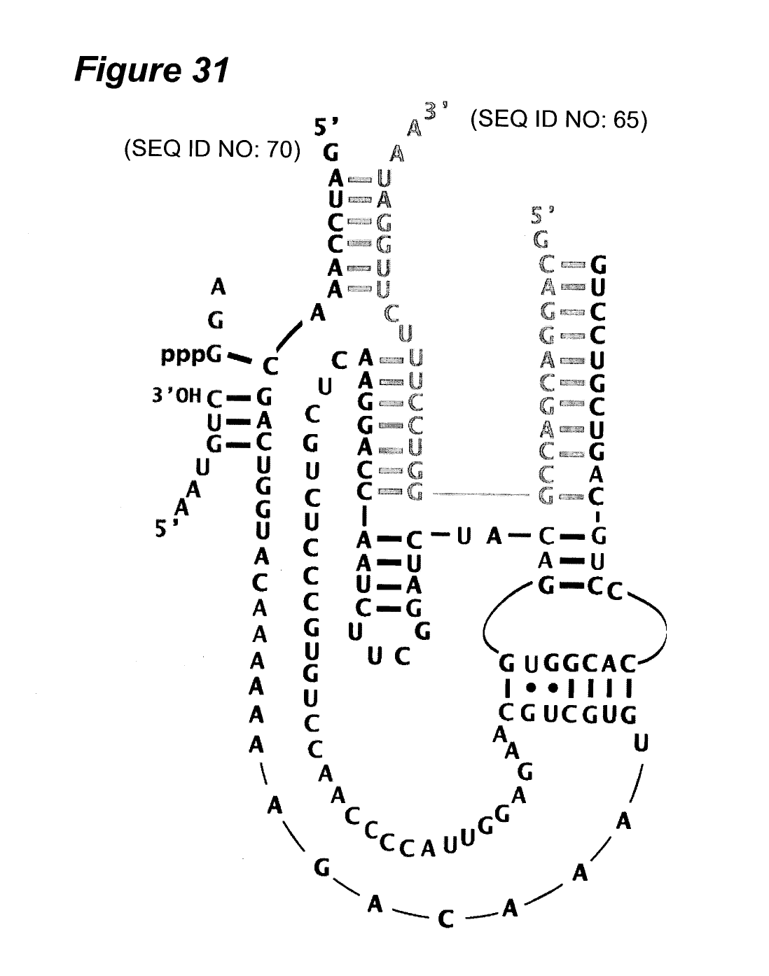

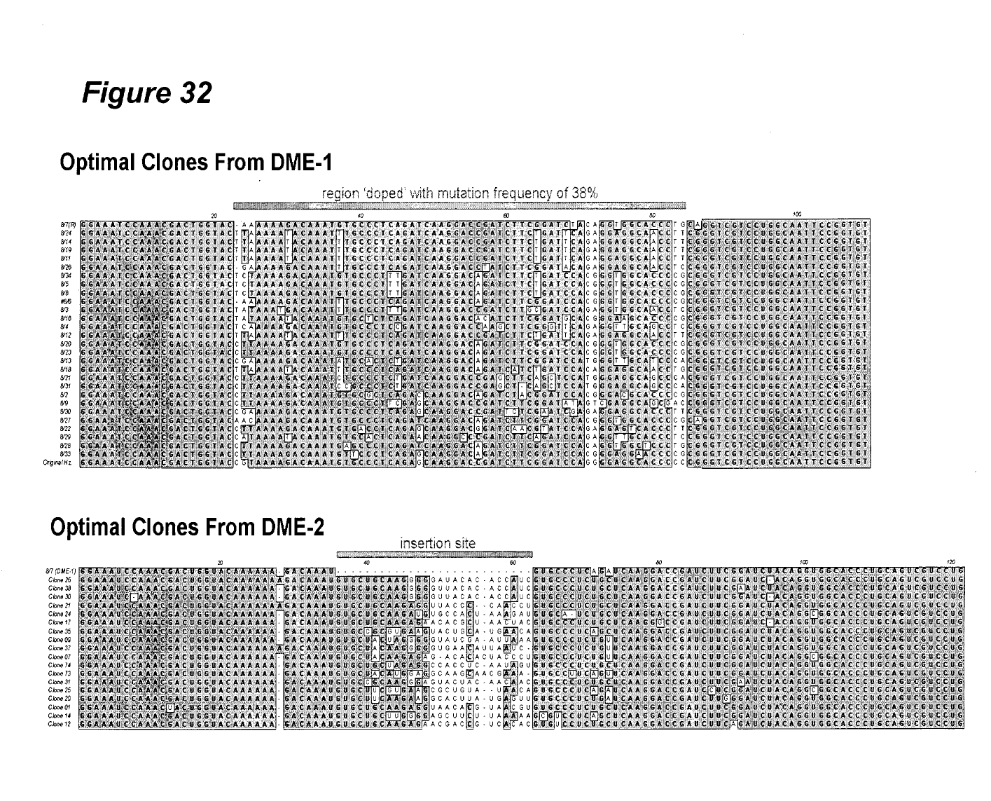

In contrast to configuration 3, HCV-Halfzyme in configuration 1 utilizes a tri-nucleotide substrate 1 that does not base pair to substrate 2, and interacts with the HCV-Halfzyme largely through non-Watson-Crick interactions. In addition, because substrate 1 and substrate 2 do not interact with each other in the absence of HCV-Halfzyme in configuration 1, uncatalyzed “background” ligation is minimized (see below). This configuration of multiple turnover HCV-Halfzyme was produced by deleting 4 nucleotides from the 5′ end of the single turnover version of clone 38 HCV-Halfzyme. 5′-pppGGA was supplied in trans as substrate 1 (FIG. 31).

Product release is the rate-limiting step for isothermal, multiple turnover, HCV-Halfzyme configuration 1 catalysis (when the standard substrate 2 is used). To increase the rate of product dissociation, a series of 5′ truncated substrate 2 RNAs were assayed for their ability to promote multiple turnover catalysis (Table V). When the interaction between substrate 2 and clone 38 HCV-Halfzyme was reduced to three Watson-Crick base pairs, the multiple turnover rate at room temperature nearly matched the rate measured for the first turnover (0. 113 min⁻¹ vs 0.4 min⁻¹, respectively). Michaelis-Menten analysis was used to establish the affinity of several of the different sequence versions of substrate 2 for the HCV-Halfzyme. This analysis suggests that the best performing substrate 2, substrate 2-4a, has an affinity of ˜11 uM for the clone 38 HCV-Halfzyme. The rate provided by saturating concentrations of substrate 2-4a is ˜150 fold reduced relative to the autoligation event promoted by this HCV-Halfzyme. In part, this reduction in rate is due to the use of sub-saturating concentrations of pppGGA (substrate 1). As with configuration 3, additional optimization is carried out so that the multiple turnover rate afforded by configuration 1 matches the autoligation rate. In this regard, the affinity of the pppGGA substrate, or variants thereof, is increased experimentally for the HCV-Halfzyme.

TABLE V

| Substrate 2 | Sequence¶ | kobs (min⁻¹) | KRNA (μM) |
|---|---|---|---|
| standard sub | aaACCAGUC | 0.0004¥ | |
| substrate 2-1 | CCAGUC | 0.006§ | |
| substrate 2-2 | CAGUC | 0.056§ | |
| substrate 2-3 | AGUC | 0.086 | 12 |
| substrate 2-4 | GUC | 0.059§ | |
| substrate 2-3a | aAGUC | 0.087£ | 15 |
| substrate 2-4b | UAGUC | 0.080£ | 15 |
| substrate 2-4c | uaaAGUC | 0.072£ | 15 |
| substrate 2-4a | AauGUC | 0.113£ | 11 |

¶lower case letters denote nucleotides that do not base pair with HCV-Halfzyme but act to destabilize the interaction of substrate 2 anf HCV-Halfzyme.
¥observed rate in presence of 20 μM substrate RNA
§observed rate in presence of 200 μM substrate RNA
£true multiple turnover rates derived from Michaelis-Menten analysis

A L.O.D. lower than 1×10⁶ HCV molecules was established using the following conditions: 4 mM pppGGA, 0.5 uM HCV-Halfzyme, pH 8, and <100 nM substrate 2-4a. Importantly, in reactions that lacked HCV-effector but were otherwise identical, no ligation could be detected after an incubation of 110 hours. Therefore, the rate of ligation in the absence of effector in configuration 1 must be less than 3×10⁻⁹ min⁻¹ (at pH 8). Given that the HCV-Halfzyme in the presence of effector has a rate of 0.113 min⁻¹, this gives a +effector/−effector rate differential greater than 3.8×10⁷. This rate differential is at least equal to the +effector/−effector rate differential afforded by configuration 3. Consequently, in so far as the rate differential controls L.O.D., the true L.O.D. afforded by configuration 1 is predicted to be as good or better than the L.O.D. provided by configuration 3. Thus, the very low extent of ‘background’ RNA ligation suggests that multiple turnover HCV-Halfzymes in configuration 1, or variants of configuration 1, may ultimately provide more sensitive detection of nucleic acids than Halfzymes in multiple turnover configuration 3.

HCV-Halfzyme Development: Conclusions

Applicant has developed HCV-activated Halfzymes and to determine their Limit of Detection (L.O.D.). This study entailed the production of HCV-Halfzymes derived from successive Directed Molecular Evolution processes, their biochemical characterization, the construction of multiple turnover HCV-Halfzymes, and their use in limit of detection (L.O.D.) determinations.

The HCV-Halfzymes that were ultimately obtained from DME display autoligation rates (ligation of substrate 2 to their own 5′ end) that are indistinguishable from the constitutively active class I ligase upon which they are based. The observed rate of this reaction is too fast to measure under the conditions used for L.O.D. determinations (>15 min⁻¹), and could equal class I ligase (˜120 min⁻¹ under these conditions). Thus, DME produced HCV-Halfzymes that were extremely efficient at performing autoligation reactions.

Several different configurations of multiple turnover HCV-Halfzymes were developed from the efficient HCV-Halfzymes obtained in DME. Of these, two configurations (1 and 3) were characterized in detail. The two configurations have very different properties. In configuration 3, the two substrates interact with one another through Watson-Crick interactions in the absence of HCV-Halfzyme. These two substrates associate with the effector bound HCV-Halfzyme by forming Watson-Crick base pairs with the HCV effector. In configuration 1, the two substrates do not interact with one another. Substrate 2, but not substrate 1, forms a stable complex with the HCV-Halfzyme.

The multiple turnover rates of these HCV-Halfzyme configurations are 0.75 min⁻¹ (configuration 3) and 0.1 min⁻¹ (configuration 1). Thus, the time required for single catalytic cycles in the two configurations is 1.4 minutes and 10 minutes, respectively. Optimization are to maximize these multiple turnover rates was underway. Multiple turnover HCV-Halfzyme catalysts that function with a rate identical to the autoligation reaction would produce>15 products per minute.

These two configurations of multiple turnover HCV-Halfzyme differ dramatically in the rate of background ligation that they promote. In configuration 3, this rate is 3.2×10⁻⁹ min⁻¹ at the conditions used for L.O.D. determinations. In contrast, the substrate RNAs used for HCV-Halfzymes in configuration 1 show absolutely no background ligation (indicating a rate of no more than 1×10^{-X} min⁻¹).

L.O.D. determinations conducted at RPI utilized substrate RNAs that were partially labeled with 32P, gel electrophoresis to resolve ligated product from substrate, and phosphoimage analysis for the direct visualization and quantification of ligated product. Using conditions that optimize detectable signal rather than turnover rate, the configuration 3 HCV-Halfzyme yielded an average 2SD L.O.D. of 1800 molecules.

Materials and Methods

RNA Synthesis

Substrate 2 RNAs were produced through standard oligoribonucleotide synthesis procedures. 5′ triphosphorylated substrate 1 oligoribonucleotides were made either by in vitro T7 RNA polymerase transcription of a corresponding DNA template, or by organic synthesis (procedure described below). Halfzymes were produced by in vitro T7 RNA polymerase transcription from DNA templates. DNA templates were either generated from PCR of an existing Halfzyme construct, or from two overlapping anti-parallel DNA oligonucleotides that were first extended to completion with Taq polymerase.

Preparation of 5′-Triphosphorylated RNA

Oligonucleotide 5′-triphosphates were prepared by subjecting solid support bound oligonucleotide to the conditions used for the preparation of nucleoside 5′-triphosphate (19). Modifications to this procedure (described below) were introduced in order to make it suitable for synthesis on oligoribonucleotides attached a to solid support.

Organic Synthesis:

1. Dry 2.5 uM synthesis column at 35° C. under high vacuum for 2 h.

2. Wash column with dry pyridine (10 mL) followed by dry DMF (10 mL).

3. Slowly push through the column 2 mL of freshly prepared solution of salicyl chlorophosphite (0.81 g) in dioxane-pyridine-DMF (2.5:1:0.5, 4 mL) for 8 min. Discard the solution and repeat the above procedure with 2 mL fresh of reagent. Total time: 16 min.

4. Wash column with dioxane (10 mL), followed by acetonitrile (10 mL).

5. Slowly push through the column 2 mL of well mixed 0.5 M P₂O₇⁴⁻.1.5 Bu₃N (Sigma, 0.712 g) in DMF-Bu₃N (3:1, 4 mL) for 10 min. Discard the solution and repeat the above procedure with fresh 2 mL of reagent. Total time—20 min.

6. Wash column with DMF (10 mL), followed by acetonitrile (10 mL).

7. Push through the column 2 mL of oxidation solution (3 g I₂ in H₂O-pyridine-THF 2:20:75) for 20 min.

8. Wash column with 70% pyridine-water (10 mL), acetonitrile (2×10 mL) and THF (10 mL). Dry with air or under vacuum.

Deprotection:

Base deprotection: 2 mL of conc. ammonia, 5 h (60° C.

TBDMS cleavage: 2 mL of 1M TBAF (Aldrich) (dried for 3 days over activated 4A molecular sieves), 16 h. Quench with 5 mL 1.5 M sodium acetate (pH 5.2). THF removed in vacuo, aq. layer extracted twice with ethyl acetate. Precipitation of the product with 20 mL of ethanol, followed by centrifugation at 16000×g produced a pellet.

Gel Electrophoresis

Denaturing Gel Electrophoresis

Denaturing, 20% acrylamide gels (19:1 acrylamide:bis-acrylamide) were run at room temperature at a constant power of 50 Watts for approximately 3 h in 1× TBE buffer (90 mM Tris-Borate, 4 mM EDTA). Gels were pre-run for approximately for 0.5 h before loading the samples in an equal amount of gel loading dye (95% formamide, 10 mM EDTA, 0.003% bromophenol blue and xylene cyanol). After running and disassembly, gels were dried and used to expose Molecular Dynamics Phosphoimager cassettes. The intensity of radiolabeled RNA was determined using Imagequant software (Molecular Dynamics).

Non-Denaturing Gel Electrophoresis and RNA-RNA Affinity Determinations

Ten percent non-denaturing acrylamide gels (19:1 acrylamide:bis-acrylamide) were run at a constant power of 50 Watts for approximately 5 h and used the following buffer conditions: 50 mM Tris-HCl pH 7.5, 0.6 mM EDTA, 30 or 60 mM MgCl2. The temperature of the gel was held constant at 23° C. by using an antifreeze coolant coil placed in the buffer chamber adjacent to the gel and the pH was maintained at 7.5 by constant circulation of the buffer between the upper and lower chambers of the gel apparatus using a peristaltic pump. In each experiment, one of the two RNAs was 5′-end radiolabeled and used in trace amounts while the second RNA varied in concentration. Binding reactions were allowed to equilibrate and loaded directly onto pre-assembled native gels. After running and disassembly, gels were used to expose Molecular Dynamics Phosphoimager cassettes. The intensity of complexed and uncomplexed radiolabeled RNA was determined using Imagequant software (Molecular Dynamics). The affinity of RNA-RNA interactions was determined using KaleidaGraph software and data fit to the equation: fraction bound=[non-labeled RNA]/(KD[non-labeled RNA]), where [non-labeled RNA] represents the RNA that varied in concentration.

Apparatus Set Up

Two plates of glasses, spacers and a comb were wiped by 95% EtOH. 1/150 volume of 10% APS and 1/1500 volume of TEMED were added to x% acrylamide in 7M Urea-1×TBE. After mixing those, the acrylamide solution was thrown into the glass plates as soon as possible, and the comb was put into. 30-minutes later, the comb was taken off and the gel was pre-run by 1× TBE. After pre-running, samples, which were mixed loading dye (e.g. 9 5% Formamide, 10 mM EDTA, 0.03% BPB, 0.03% XP), were applied on the gel. After running, a gel was quantitated by phosphoimage analysis. For example, positions of substrate 2 and product are as a right picture on 15% acrylamide gel.

Kinetic Assays

Kinetic assays were performed at 23° C. in 30 mM buffer at 3 mM to 120 mM MgCl2 as specified. Solutions were buffered with MES (pH 5.5, 6.0, 6.5) or Tris-HCl (pH 7.0, 7.5, 8.0, 8.5, 9.0). In all assays, the HCV-Halfzyme and effector were heated at 80° C. for two minutes, 5× buffer was immediately added, and the reaction allowed to cool to 23° C. over 5 minutes. Reactions were initiated by addition of substrate RNAs.

Single Turnover Kinetic Assays

A trace concentration of 5′-32P labeled Substrate 2 (<5 nM) was incubated with 1 uM HCV-Halfzyme. Time points were taken from 5 sec. to 30 minutes depending on the catalytic rate of the HCV-Halfzyme. Single turnover observed rates were determined by fitting the quantified data either to a linear equation (fraction ligated versus time) or to the single exponential equation: fraction ligated=Fa(1-e-kt), where t equals time, k equals rate of catalysis, and Fa equals the fraction of ligated substrate 2 at completion. Data was fit using Kalidagraph (Synergy Software).

Multiple Turnover Kinetic Assays

Turnover rates were calculated from the initial rate of the reaction (<20% substrate converted to ligated product) and fit to the following equation: ([ligated product]/[32P substrate 2]*[HCV-Halfzyme]). When required, Michaelis-Menten parameters were established by varying substrate concentration and fitting to the Michaelis-Menten equation: Data were fit to the equation: v=[E][S]kcat/(KM+[S]), where v equals rate at each [S], S represents substrate 1 concentration (the concentration of substrate 2 was always equal to substrate 1 concentration), KM equals apparent binding constant for half-maximal activation. Conditions for the different configurations of multiple turnover HCV-Halfzymes are described below.

TABLE VI

| | | | |
|---|---|---|---|
| | | No HZ, | |
| +Effector | −Effector | No Eff | Final conc. |

Solution 1

| | | | | |
|---|---|---|---|---|
| Halfzyme (5 uM) | 2 | 2 | — | 0.5 uM |
| Effector (5 uM) | 2 | — | — | 0.5 uM |
| H₂O | 1.9 | 3.9 | 5.9 | |

Heat 80 degree for 2 mins., cool to 22 degree for 5 mins.

| | | | | |
|---|---|---|---|---|
| 5 x reaction buffer | 4 | 4 | 4 | 1x |

Solution 2

| | | | | |
|---|---|---|---|---|
| substrate 1 | 5 | 5 | 5 | |
| substrate 2 | 5 | 5 | 5 | |
| ³²P-substrate 2 | 0.1 | 0.1 | 0.1 | Trace |
| Total | 20 | 20 | 20 | |
| | (ul) | (ul) | (ul) | |

Configuration 1: Unless otherwise noted, assays were carried out at 4 mM pppGGA, <5 nM to 25 uM of 5′-32P labeled substrate 2, 0.5 uM HCV-Halfzyme, 40 mM MgCl2, and 30 mM Tris-HCl (pH 8.0). Data points were taken from 1 h to 48 h.

Configuration 3: Assays were carried out with equal concentrations of substrate 1 and substrate 2 (at 0.5 uM), trace 5′-32P labeled substrate 2, 0.5 HCV-Halfzyme, 60 or 120 mM MgCl2 at pH 6.5 or 7.5. Data points were taken from 0.5 h to 20 h.

Limit of Detection (L.O.D.) Determinations

L.O.D. determinations were carried out at 23° C. in 30 mM buffer, 0.6 mM EDTA, and at different pHs and concentrations of MgCl2 depending on the particular configuration of multiple turnover HCV-Halfzyme (see below). Assays contained 0, 60, 180, 600, 1800, 6000, 1.8×104, 1.8×105, 1.8×106 or 1.8×107 HCV effector molecules, which were serially diluted into 100 ng/μL yeast tRNA from a concentrated stock solution of synthetic HCV-effector oligonucleotide (concentration determined by OD260 and the extinction coefficient of the HCV effector oligonucleotide, ε=2.8324×105 M−1 cm−1).

In all assays, HCV-Halfzyme (0.5 uM) and effector nucleic acid were heated together at 80° C. for two minutes, 5× buffer immediately added, and allowed to cool to 23° C. over 5 minutes. Reactions were initiated by addition of both substrate RNAs. When the level of HCV effector approached the L.O.D. of the HCV-Halfzyme reactions were performed at n=5. Control reactions in which HCV effector nucleic acid was omitted were also performed at n=5.

Configuration 1: Assays were carried out at 1 mM pppGGA, 12.5 uM Substrate 2-4a, 0.5 uM HCV-Halfzyme, effector, 30 mM Tris-HCl (pH=8.0), 120 mM MgCl2, and 0.6 mM EDTA. Reaction time was 65 h.

Configuration 3: Assays were carried out at 0.5 uM of Substrate 1 and Substrate 2, 0.5 uM HCV-Halfzyme, effector, 30 mM Tris-HCl (pH=7.5), 60 mM MgCl2, and 0.6 mM EDTA. Reaction time was 45 h.

TABLE VII

| | | |
|---|---|---|
| +Effector | −Effector | Final conc. |

Solution 1

| Halfzyme (5 uM) | 0.25 | 0.25 | 0.5 uM |
|---|---|---|---|
| Effector | 1 | — | |
| tRNA (100 ng/ul) | — | 1 | |
| H₂O | 1.55 | 2.55 | |
| Heat 80 degree for 2 mins., cool to 22 degree for 5 mins. | | | |
| 5 x reaction buffer | 1 | 1 | 1x |

Solution 2

| | | |
|---|---|---|
| substrate 1 | 0.5 | 0.5 |
| substrate 2 | 0.5 | 0.5 |
| ³² P-substrate 2 | 0.2 | 0.2 |
| Total | 5 | 5 |
| | (ul) | (ul) |

Directed Molecular Evolution

DME-1:

Library Construction:

The pool for DME-1 was derived from the Class-1 ligase. The pool contained a central region of 62 positions mutagenized to 30% and flanked on both sides by constant sequence region (5′-ACACCGGAATTGCCAGGACGACCGgggggtgcctcccctggatccgaagatcggtccttgctctgaggg cacatttgtcttttacgGTACCAGTCGTTTGGATTTCC-3′) (SEQ ID NO: 82). The pool was amplified in a 5 mL PCR reaction using primers that added the promoter sequence for T7 RNA polymerase (5′-GCTAATACGACTCACTATAGGAAATCCAAACGACTGGTAC-3′ (SEQ ID NO: 83) and 5′-ACACCGGAATTGCCAGG-3′, (SEQ ID NO: 84)). The final complexity of the population was 1×10¹⁰. One nmole of the pool DNA was transcribed with T7 polymerase in a 2 mL reaction and RNA purified on a 10% polyacrylamide gel.

Selection:

Selection was carried out starting with 2×1015 molecules (4 nmoles). Pool RNA and 1.1 equivalent of effector RNA (5′-ACACCGGAAUUGCCAGGACGACCGGGUCCUUUCUUGGAUAA-3′, (SEQ ID NO: 85)) was heated in water to 80° C. for 3 min. and cooled to room temperature for 10 min. 2× selection buffer was added (final buffer conditions: 30 mM Tris (pH 7.5), 200 nM KCl, 0.6 mM EDTA and 60 mM MgCl2) along with 2.2 equivalents of a substrate 2 variant that allowed ligation product-specific PCR. After a define period of incubation, the reaction was stopped by the addition of EDTA, HCV-Halfzyme ligated to substrate 2 was purified on a 10% denaturing acrylamide gel. The selected RNA was amplified by RT-PCR and the T7 promoter was restored through a nested PCR. A total of 8 rounds of DME were performed. At each, the selection stringency was increased by progressively decreasing the ligation time from 16 h to 15 sec. in round 8th. A negative selection step of 20 h incubation in the absence of effector of 20 h was introduced in round 4th to prevent the amplification of effector-independent ligases.

DME-2

Library Construction:

The pools for DME-2 were constructed based on the 8/7 HCV-Halfzyme from DME-1. Three libraries were constructed in which random sequences of either 30 or 26 nucleotides were inserted at different positions (library-1, 5′-CCAGGACGACTGCAGGGTGCCACCTGTAGATC(N30)GATCGGTCCTTGATCTG AGGGCACATTTGTCTTTTTTG-3′ (SEQ ID NO: 86); library-2, 5′-GGAAATCCAAACGACTGGTACAAAAAAGACAAAT(N26)GTGCCCTCAGATCA AGGACCGATCTTCGGATCTACAGG-3′ (SEQ ID NO: 87); library-3, 5′-GGAAATCCAAACGACTGGTACAA(N26)AAAAGACAAATGTGCCCTCAGATCA AGGACCGATCTTCGGATCTACAGG-3′, (SEQ ID NO: 88)). Each library was amplified in a 5 mL PCR reaction using primers that extended the 5′ constant region and added the promoter sequence for T7 RNA polymerase (5′-GCTAATACGACTCACTATAGGAAATCCAAACGACTGGTACAAAAAAGACAA ATGTGCC-3′, (SEQ ID NO: 89); 5′-CCAGGACGACTGCAGGGTGCCACCTGTAGATCCGAAGATCGGTCC-3′, (SEQ ID NO: 90); 5′-GCTAATACGACTCACTATAGGAAATCCAAACGACTGGTAC-3′ (SEQ ID NO: 91) and 5′-CCAGGACGACTGCAGGGTGCC-3′, (SEQ ID NO: 92)). The final complexity of the each population was ˜3×1014. 0.6 nanomole of the each pool was transcribed separately with T7 RNA polymerase in a 1 mL reaction and RNA purified on a 10% polyacrylamide gel.

Selection:

Selection was carried out starting with 1×1015 molecules for each DME. Each library was subjected to selection at pH 6.0 (MES) and pH 7.5 (Tris-HCl). Library RNA and 1.1 equivalent of effector RNA (5′-CCAGGACGACCGGGUCCUUUCUUGGAUAA-3′, (SEQ ID NO: 93)) was heated in water to 80° C. for 3 min. 4× selection buffer was immediately added along with MgCl2 to bring the buffer conditions at 30 mM Tris pH 7.5 (or MES pH 6.0), 0.6 mM EDTA and 0.1% NP40. A total of 8 rounds of DME were performed. MgCl2 was progressively decreased to increase the stringency of selection (20 mM in round 1 to 3 mM in round 8). Selection stringency was also increased by progressively decreasing the ligation time from 10 min. to 5 sec. in the round 8th. Reactions were started by the addition of substrate 2. Selected RNA was amplified as in DME-1. A negative selection step of 20 h was introduced in rounds 5, 7 and 8 to prevent the amplification of effector-independent ligases. Libraries from DME-2s conducted at pH 6 and pH 7.5 were mixed separately after round 7 and made to compete against one another.

Cloning and Sequencing

To identify individual clones, DNA from the final cycle of DME was cloned into E. coli (TOP10) using TOPO TA cloning kit according to manufacturer's instructions (Invitrogen). Cloned DNA from individual colonies was amplified by the colony PCR method using M13 forward and M13 reverse primers. Both strands of each clone were PCR sequenced using dideoxy-terminated sequencing and fluorescent dyes (ABI). Sequencing reactions were analyzed on an ABI Prism 310 Genetic Analyzer and sequence alignments performed using DS Gene software.

### Example 14

### Zeptomole Detection of HCV RNA Using an Optimized HCV-Halfzyme Nucleic Acid Sensor Molecule

Applicant further optimized the HCV halfzyme constructs described in Example 13 above. Following the sequence optimization of substrate RNAs, this HCV-activated half ribozyme displayed a maximal turnover rate of 100 min⁻¹ (pH 8.3) and was induced in rate by approximately 3.75 billion-fold relative to the uncatalyzed reaction. This half ribozyme was able to detect the HCV effector in the zeptomole range (˜6700 molecules), a sensitivity of detection roughly 2.7 million-fold greater than that previously demonstrated by oligonucleotide-activated ribozymes and one sufficient for molecular diagnostic applications.

Optimization of Substrate RNA Utilization Improves Turnover Rate

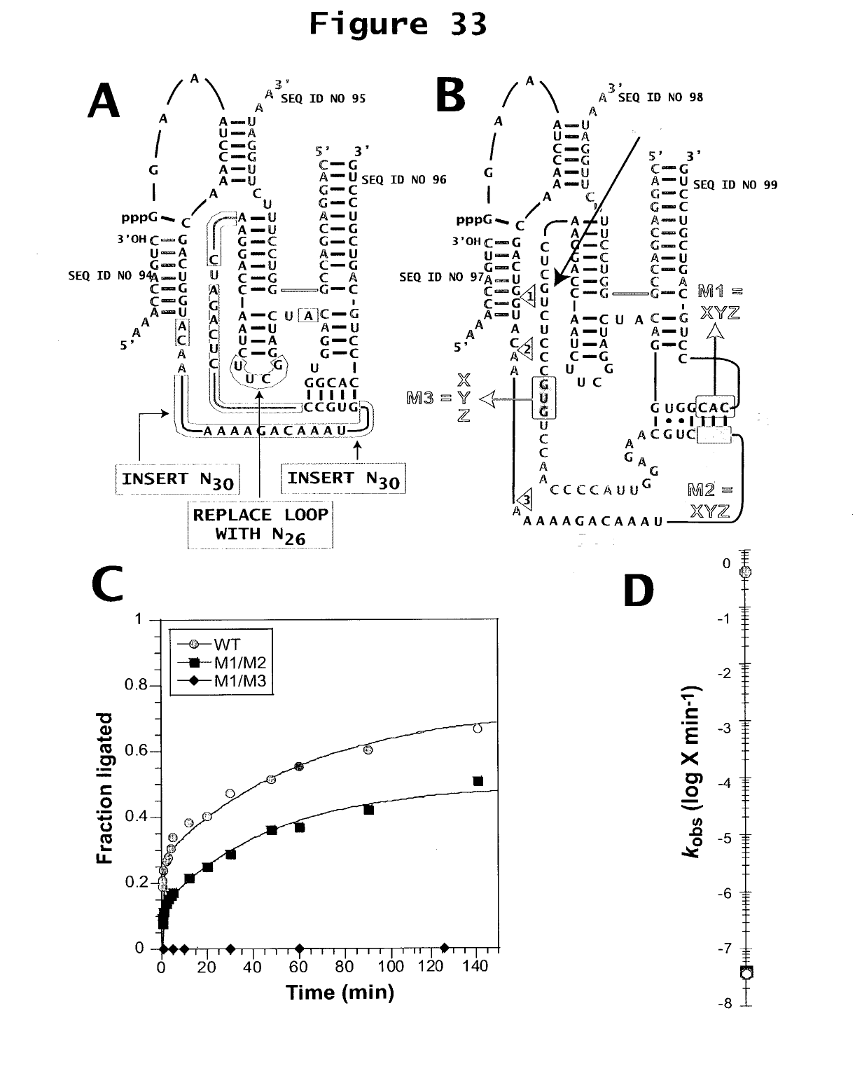

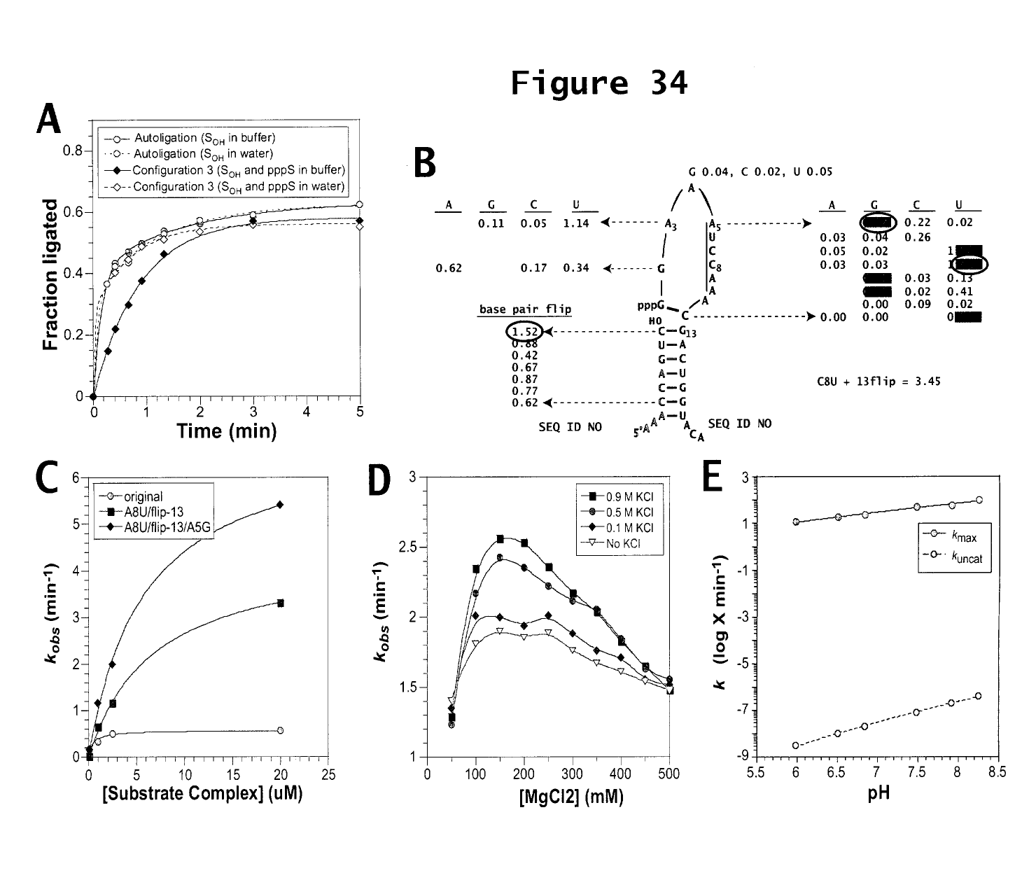

Multiple turnover rates of the clone 21 Halfzyme in the presence of a quantitatively bound, stoichiometric amount of effector (FIG. 33D) were more than 17-fold lower than its observed rate of autoligation (FIG. 33C). To examine whether this decrease reflected a rate limiting step that occurred before or after the first catalytic cycle, the observed rate of ligation was determined for the configuration 3 multiple turnover clone 21 Halfzyme in a single turnover regime when bound to effector (FIG. 34A). The first catalytic cycle of the multiple turnover Halfzyme proceeded with a rate that was too fast to accurately measure, and indistinguishable from the rate of autoligation, when the two substrates were annealed in water and added to the Halfzyme effector complex. Unexpectedly, the rate of the first turnover decreased to 1.13 min⁻¹ if the substrate RNAs were pre-equilibrated in reaction buffer. Further studies identified MgCl₂ as the buffer component responsible for this phenomenon. In contrast, the rate of autoligation was not compromised after pre-equilibration of the 5′ substrate in reaction buffer.

Interestingly, if the turnover rate is calculated from the fraction of HCV-Halfzyme active for multiple turnover catalysis (58% active, FIG. 34A)—not the total Halfzyme concentration—the resultant multiple turnover rate is essentially identical to the rate of the first catalytic cycle seen with substrate RNAs pre-equilibrated in buffer [1.2 min⁻¹ (FIG. 33D) versus 1.13 min⁻¹ (FIG. 34A), respectively]. Thus, the reduced rate of multiple turnover catalysis relative to autoligation could result from a MgCl₂-dependent phenomenon slowing utilization of the substrate RNA complex—not from issues relating to the functional characteristics of the clone 21 Halfzyme itself.

In an effort to abrogate this affect, mutant substrate RNAs were produced and the turnover rates they afford to the clone 21 Halfzyme were determined (FIG. 34B). Forty six mutant substrates were tested. Only two [C8U in P2 and a base pair “flip” in P1 that exchanges the identity of the 3′-most nucleotide of S_{OH} and its pairing partner in pppS (flip-13)] afforded turnover rates greater than the original substrate pair (an increase of 1.45-fold and 1.52-fold, respectively). Notably, a S_{OH} and pppS substrate RNA pair that contained both C8U and flip-13 mutations afford a turnover rate that was slightly greater than the sum of the two individual mutants, resulting in a turnover rate that was 3.45-fold greater than the initial substrate pair (FIG. 34B).

The mutant substrate RNA pairs also provided information concerning the recognition of the substrate complex by the Halfzyme. For example, P2 tolerated G-U wobble base pairs only in some locations. As expected, disruption of P2 base pairs greatly diminished activity and P1 base pair “flips” (besides flip-13) had only small (negative) effects on activity. Position C12, the pppS position that forms an intramolecular Watson-Crick base pair with G1 and serves to localize the reactive triphosphate next to the attacking nucleophile, could not be mutated even if it allowed wobble pairing to G1. Positions A11 and A4, both highly conserved in the constitutively active Class I ligase (Ekland and Bartel, 1995, Nucleic Acids Res., 23, 3231-3238), were intolerant to sequence change. In contrast, mutation of A3, another highly conserved position in the Class I ligase (Ekland and Bartel supra), either decreased (A3G and A3C) or slightly increased (A3U) activity. Unlike its importance in the constitutive Class I ligase (Ekland and Bartel supra), the most severe decrease in activity displayed by mutation of G2 was a 0.34-fold reduction. Thus, this analysis suggests that the HCV-Halfzyme does not recognize and bind to its substrate RNA complex in a manner identical to the constitutively active Class I ligase.

Since the optimal substrate RNA pair (C8U/flip-13) carried a change in P2 that could effect its affinity for the Halfzyme/effector complex, its activity as a function of substrate RNA concentration was compared to the original substrate RNA pair (FIG. 34C). Significantly, the multiple turnover rate of the clone 21 Halfzyme in the presence of effector did not appreciably change as the concentration of the original substrate pair was varied between 100 nM to 20 uM. In contrast, the turnover rate of the clone 21 Halfzyme-effector complex did respond to the concentration of the C8U/flip-13 substrate pair, displaying a turnover rate of 3.33 min⁻¹ at 20 uM substrate RNA. Since the concentration of the C8U/flip-13 substrate pair required to promote maximal activity was not reached, we concluded that the C8U/flip-13/A5G substrate pair had a reduced affinity for the effector-Halfzyme complex relative to the original substrate pair. In an attempt to increase substrate RNA affinity, applicant tested the activity as a function of concentration of a triple mutant (C8U/flip-13/A5G) that was predicted by an RNA folding program (Xia, et al., 1998, Biochemistry, 37, 14719-14735) to have an increased affinity (0.5 kcal/mol) for the effector relative to the C8U/flip-13 substrate pair. Indeed, the triple mutant showed an increase in activity relative to the double mutant at all substrate RNA concentrations examined. Thus, further efforts in optimizing catalytic rate were focused on the C8U/flip-13/A5G triple mutant substrate RNA pair.

The LOD provided by Halfzymes is dictated by the rate differential between the ribozyme catalysis in the presence of effector and the uncatalyzed reaction (see below). Therefore, it was important to minimize the amount of substrate RNA in reactions because the amount of product formed due to uncatalyzed ligation will scale with substrate RNA concentration. To identify conditions that increase the affinity of the C8U/flip-13/A5G substrate RNA pair and therefore increase Halfzyme activity at lower concentrations of substrate RNA (increase in k_{cat}/Kₘ), the kinetic performance of the Halfzyme was examined. The concentration of monovalent and divalent metal ions in Halfzyme reactions were varied since the ionic strength and the concentration of specific metals can influence molecular association and/or RNA folding. This screen indicated that turnover rate increased as ionic strength increased (compare k_{obs} at different KCl concentrations every MgCl₂ concentration, FIG. 34D). Optimal MgCl₂ concentration was ˜150 mM; observed rate was slower when the MgCl₂ concentration was either less or greater than this amount. Applicant interpreted these data to reflect both a magnesium ion-specific effect and an ionic strength effect on rate. At optimal salt concentrations (0.9 M KCl, 150 mM MgCl₂) the turnover rate of the Halfzyme increased to 2.56 min⁻¹ at 1 uM substrate complex—a 2.2-fold increase relative to the original buffer condition (FIG. 34C).

Using the C8U/flip-13/A5G substrate RNA pair and the optimized metal ion concentrations, k_{obs} was determined as a function of substrate RNA concentration and pH. Lineweaver-Burk analysis of these data was used to generate Kₘ and kₘₐₓ at each pH. Similar to the constitutive ligase, Kₘ of the substrate complex varied little from pH 6.0 to pH 8.25 (from 5 uM to 16 uM). When adjusted for the fraction of active Halfzyme (58%), kₘₐₓ is predicted to be 100 min⁻¹] at the highest pH tested (pH 8.3, FIG. 34E). Maximal rate did not display a log-linear relationship with pH, but instead increased roughly 1.5-fold per pH unit (FIG. 34E). Thus, in contrast to the constitutive Class I ligase, the multiple turnover rate of ligation of the C8U/flip-13/A5G substrate RNA pair by the clone 21 Halfzyme is not limited solely by a hydroxide ion-dependent chemical step. At pH 6.0 and 6.5, the maximal turnover rate (12 min⁻¹ and 19 min⁻¹, respectively) was greater than the rate of autoligation or multiple turnover catalysis promoted by the constitutive Class I ligase at these pH values (˜3 min⁻¹ and ˜10 min⁻¹, respectively, (Bergman et al., supra; Glasner et al., 2002, Biochemistry, 41, 8103-8112).

The uncatalyzed rate of ligation of the C8U/flip-13/A5G substrate RNA pair was also determined as a function of pH (FIG. 34E). As expected, the rate of the uncatalyzed reaction was independent of substrate RNA concentration and increased log-linear with pH. The difference in the rate of substrate RNA ligation in the presence versus absence of effector, therefore, was maximal at the lowest pH tested. It ranged from 3.75 billion-fold at pH 6.0 to 240 million-fold at pH 8.3.

Limit of Detection of HCV Effector Sequence

The LOD of the clone 21 HCV-Halfzyme was estimated based on the kinetic analysis performed above. Since Halfzymes are devoid of catalytic activity in the absence of target oligonucleotide, the sensitivity of detection of an effector oligonucleotide present in low amounts can be judged from the difference in the rate of product formation due to effector-activated Halfzyme catalysis and the rate of product formation through uncatalyzed ligation. Both rates can be derived from rate equations; the former is given by:

k_{cat}[effector-Halfzyme] 1

while the latter is given by:

k_{uncat}[substrate complex] 2

If the concentration of effector-Halfzyme is taken to equal the total concentration of effector (and will be if the effector is quantitatively captured) then effector increases the amount of ligation product two-fold above that seen in its absence when:

k_{cat}[effector-Halfzyme]=k_{uncat}[substrate complex] 3

Since k_{uncat} is known (FIG. 34E) and k_{cat} at any substrate RNA concentration is defined by experimentally determined kₘₐₓ and Kₘ parameters using the Michaelis-Menten relationship:

k_{cat}=[substrate complex]/([substrate complex]+Kₘ)*kₘₐₓ 4

Equation 3 can be solved for the concentration of effector-Halfzyme; which is the total concentration of effector at the limit of detection:

[effector-Halfzyme]=k_{uncat}[substrate complex]/{[substrate complex]/([substrate complex]+Kₘ)*kₘₐₓ} 5

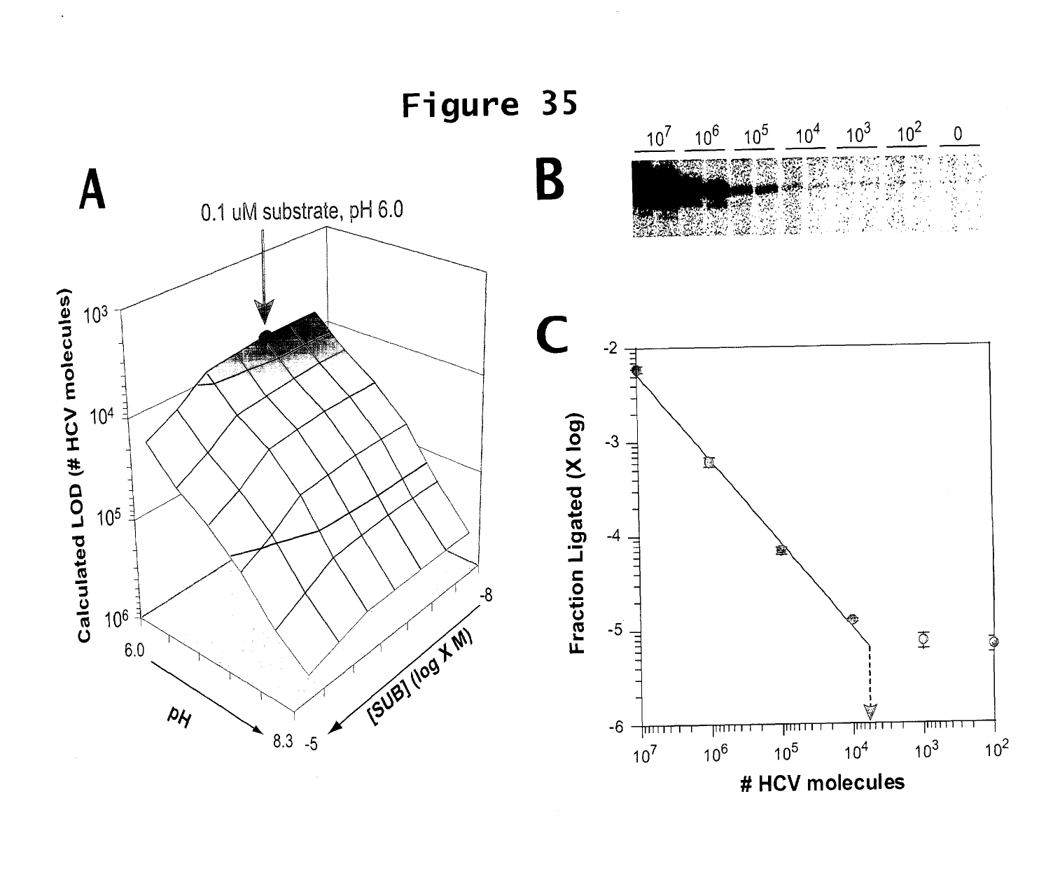

Equation 5 was used to calculate LOD as a function of substrate RNA concentration at various pH values (FIG. 35A). Here, the LOD is defined as one half of the concentration of effector-Halfzyme that supports a rate of product formation equal to that of the uncatalyzed ligation, i.e., when the amount of ligation product in the presence of effector is indistinguishable from the amount of product produced in its absence.

As expected, the calculated LOD improves as pH is lowered since the uncatalyzed reaction displays a logarithmic increase with pH but the catalyzed reaction does not (FIG. 34E). Interestingly, the calculated LOD suffers as substrate RNA concentration approaches and exceeds Km because the amount of product formed through uncatalyzed ligation increases more than the increase in product formation provided by the enhanced rate of Halfzyme catalysis. As substrate RNA concentration is lowered, the calculated LOD asymptotically approaches 6910 effector molecules. Thus, the calculated LOD is maximized at concentrations of substrate RNA that actually attenuate Halfzyme catalysis. However, since the maximal LOD is asymptotically approached as substrate RNA concentration is decreased, a substrate RNA concentration can be defined that supports significant ribozyme activity but does not appreciably compromise LOD. For example, the Halfzyme is predicted to display a turnover rate of 0.133 min⁻¹ and an LOD of 7050 HCV molecules at 100 nM substrate RNA.

To test the validity of this method of calculating the LOD, the amount of ligation product produced by the clone 21 HCV-Halfzyme was determined as a function of HCV effector concentration in 5 uL reactions carried out at pH 6.0, 100 nM substrate RNA (FIG. 35B). Ligation product above that observed in the absence of HCV oligonucleotide was clearly evident with as few as 10⁴ copies of the HCV effector oligonucleotide (1.6 fM, FIG. 35B) and Halfzyme catalysis quantitatively reported effector amounts ranging from this level to the highest amount of HCV effector tested (10⁷ molecules) (FIG. 35C). Extrapolated turnover rates in each of these reactions—values that depend on the precise number of effector molecules in each dilution—averaged 0.164±0.014 min⁻¹. The close agreement of these turnover rates to the predicted rate under these conditions obtained from Kₘ and kₘₐₓ values (0.133 min⁻¹, FIG. 34E and data not shown), together with the logarithmic decrease in product as a function of effector concentration, suggests that each serial dilution contains the indicated number of effector molecules. Data from 10⁷ to 10⁴ molecules was fit to a power (x^{y}) function (R²=0.99946) and the LOD was defined from the signal observed in the absence of effector in direct analogy to the method used to define the calculated LOD. The resultant value of 6690 HCV molecules (˜11 zeptomoles, ˜2 fM) is in remarkable agreement with the LOD calculated from the rates of Halfzyme and uncatalyzed product formation under these conditions (7050 molecules, FIG. 35A). As expected from the calculated LOD (FIG. 35A), signal observed in the presence of 1000 and 100 HCV molecules was indistinguishable from the signal observed in the absence of HCV effector oligonucleotide (FIGS. 35B,C). Thus, Halfzymes allow detection of oligonucleotide targets present in the zeptomole range in accordance with the LOD calculated from their kinetic properties.

Other Uses

The nucleic acid sensor molecules of this invention can be used as diagnostic tools to examine genetic drift and mutations within diseased cells or to detect the presence of a specific RNA in a cell. The close relationship between nucleic acid sensor molecule activity and the structure of the target RNA allows the detection of mutations in any region of the molecule which alters the base-pairing and three-dimensional structure of the target RNA. By using multiple nucleic acid sensor molecules described in this invention, one can map nucleotide changes which are important to RNA structure and function in vitro, as well as in cells and tissues. Cleavage of target RNAs with nucleic acid sensor molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple nucleic acid sensor molecules targeted to different genes, nucleic acid target molecules coupled with known small molecule inhibitors, or intermittent treatment with combinations of nucleic acid sensor molecules and/or other chemical or biological molecules). Other in vitro uses of nucleic acid sensor molecules of this invention comprise detection of the presence of mRNAs associated with a disease-related condition. Such RNA is detected by determining the presence of a cleavage product after treatment with an enzymatic nucleic acid molecule using standard methodology.

In a specific example, nucleic acid sensor molecules which cleave only wild-type or mutant forms of the target RNA are used for the assay. The first nucleic acid sensor molecule is used to identify wild-type RNA present in the sample and the second nucleic acid sensor molecule is used to identify mutant RNA in the sample. As reaction controls, synthetic substrates of both wild-type and mutant RNA are cleaved by both nucleic acid sensor molecules to demonstrate the relative nucleic acid sensor molecule efficiencies in the reactions and the absence of cleavage of the “non-targeted” RNA species. The cleavage products from the synthetic substrates also serve to generate size markers for the analysis of wild-type and mutant RNAs in the sample population. Thus, each analysis can require two nucleic acid sensor molecules, two substrates and one unknown sample, which are combined into six reactions. The presence of cleavage products is determined using an RNAse protection assay so that full-length and cleavage fragments of each RNA can be analyzed in one lane of a polyacrylamide gel. It is not required to quantify the results to gain insight into the expression of mutant RNAs and putative risk of the desired phenotypic changes in target cells. The expression of mRNA whose protein product is implicated in the development of the phenotype is sufficient to establish risk. If probes of comparable specific activity are used for both transcripts, then a qualitative comparison of RNA levels is sufficient and decreases the cost of the initial diagnosis. Higher mutant form to wild-type ratios are correlated with higher risk whether RNA levels are compared qualitatively or quantitatively.

Additional Uses

Potential usefulness of sequence-specific nucleic acid sensor molecules of the instant invention have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to detect gene expression of target genes in bacterial, microbial, fungal, viral, and eukaryotic systems including plant, or mammalian cells.

The nucleic acid sensor molecules of the invention represent a new class of therapeutic agents capable of modulating the expression of target genes, peptides, proteins, and other biologically active molecules in vivo as described herein. The therapeutic activity of nucleic acid sensor molecules of the invention can respond to both internal and external stimuli in a subject, for example the presence of a gene, pathogen, SNP, peptide, protein, RNA, metabolite, neurotransmitter, co-factor, drug, toxin, or physical stimuli such as light, gravity, temperature, and pressure.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses which are encompassed within the spirit of the invention, are defined by the scope of the claims. It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
A. 2.5 μmol Synthesis Cycle ABI 394 Instrument

| Reagent | Equivalents | Amount | Wait Time* DNA | Wait Time* 2′-O-methyl | Wait Time* RNA |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| Imidazole | | | | | |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| | | | | | |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| ICA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

C. 0.2 μmol Synthesis Cycle 96 well Instrument

| | Equivalents: DNA/ | Amount: DNA/2′-O- | | Wait Time* 2′-O- | Wait Time |
|---|---|---|---|---|---|
| Reagent | 2′-O-methyl/Ribo | methyl/Ribo | Wait Time* DNA | methyl | Ribo |
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl | 502/502/502 | 50/50/50 μL | 10 sec | 10 sec | 10 sec |
| Imidazole | | | | | |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

*Wait time does not include contact time during delivery.

1. A nucleic acid sensor molecule comprising an enzymatic nucleic acid component and a separate effector component, wherein the enzymatic nucleic acid is assembled from two or more separate nucleic acid molecules, wherein the separate effector component is one of the two or more separate nucleic acid molecules that make up the enzymatic nucleic acid component of the nucleic acid sensor molecule, such the in the presence of the separate effector component, the enzymatic nucleic acid component assembles in a form necessary to enable the nucleic acid sensor molecule to catalyze a chemical reaction involving one or more reporter molecules, and wherein the effector and the reporter molecules are separate molecules.

2. The nucleic acid sensor molecule of claim 1, wherein the chemical reaction is a ligation reaction.

3. The nucleic acid sensor molecule of claim 2, wherein the ligation reaction involves covalent attachment of a first reporter molecule to a second reporter molecule.

4. The nucleic acid sensor molecule of claim 2, wherein the ligation reaction results in the formation of a phosphodiester bond.

5. The nucleic acid sensor molecule of claim 3, wherein the first or second reporter molecule independently comprises a terminal phosphate group.

6. The nucleic acid sensor molecule of claim 1, wherein the chemical reaction is a phosphodiester cleavage reaction.

7. The nucleic acid sensor molecule of claim 1, wherein the reporter molecule comprises one or more polynucleotides.

8. The nucleic acid sensor molecule of claim 1, wherein the enzymatic nucleic acid component is assembled from two separate nucleic acid molecules.

9. The nucleic acid sensor molecule of claim 1, wherein the enzymatic nucleic acid component is assembled from three separate nucleic acid molecules.

10. A method, comprising:
(a) contacting the nucleic acid sensor molecule of claim 1 with a system under conditions suitable for the nucleic acid sensor molecule and to catalyze a chemical reaction on a reporter molecule; and
(b) assaying for the chemical reaction on the reporter molecule.

11. The method of claim 10, wherein the chemical reaction is indicative of the presence of a target nucleic acid in the system.

12. The method of claim 10, wherein the chemical reaction is indicative of the system lacking a target nucleic acid.

13. The nucleic acid sensor molecule of claim 1, wherein the effector component is an RNA or DNA derived from a bacteria, virus, fungi, plant or mammalian genome.

14. The method of claim 11, wherein the target nucleic acid is an RNA or DNA derived from a bacteria, virus, fungi, plant or mammalian genome.

15. The nucleic acid sensor molecule of claim 1, wherein the effector component comprises a sequence derived from the Hepatitis C virus (HCV).

16. The method of claim 11, wherein the target nucleic acid comprises a sequence derived from the Hepatitis C virus (HCV) 5′-UTR.

17. The nucleic acid sensor molecule of claim 15, wherein the Hepatitis C virus (HCV) sequence is derived from the 5′-UTR.

18. The method of claim 16, wherein the Hepatitis C virus (HCV) sequence is derived from the 5′-UTR.

19. A kit comprising the nucleic acid sensor molecule of claim 1.

20. A nucleic acid sensor molecule comprising an enzymatic nucleic acid component and one or more sensor components wherein, in response to an interaction of a single stranded RNA (ssRNA) having a single nucleotide polymorphism (SNP) with the nucleic acid sensor molecule in a system, the enzymatic nucleic acid component catalyzes a chemical reaction resulting in a detectable response.

21. A nucleic acid sensor molecule comprising an enzymatic nucleic acid component and one or more sensor components wherein, in response to an interaction of a single stranded DNA (ssDNA) having a single nucleotide polymorphism (SNP) with the nucleic acid sensor molecule in a system, the enzymatic nucleic acid component catalyzes a chemical reaction resulting in a detectable response.

22. A nucleic acid sensor molecule comprising an enzymatic nucleic acid component and one or more sensor components wherein, in response to an interaction of a single stranded RNA (ssRNA) with the nucleic acid sensor molecule in a system, the enzymatic nucleic acid component catalyzes a chemical reaction resulting in cleavage of a predetermined nucleic acid molecule associated with a disease.

23. A nucleic acid sensor molecule comprising an enzymatic nucleic acid component and one or more sensor components wherein, in response to an interaction of a single stranded DNA (ssDNA) with the nucleic acid sensor molecule in a system, the enzymatic nucleic acid component catalyzes a chemical reaction resulting in cleavage of a predetermined nucleic acid molecule associated with a disease.

24. A nucleic acid sensor molecule comprising an enzymatic nucleic acid component and one or more sensor components wherein, in response to an interaction of a single stranded RNA (ssRNA) having a single nucleotide polymorphism (SNP) with the nucleic acid sensor molecule in a system, the enzymatic nucleic acid component catalyzes a chemical reaction resulting in ligation of a predetermined nucleic acid molecule to another predetermined nucleic acid molecule.

25. A nucleic acid sensor molecule comprising an enzymatic nucleic acid component and one or more sensor components wherein, in response to an interaction of a single stranded DNA (ssDNA) having a single nucleotide polymorphism (SNP) with the nucleic acid sensor molecule in a system, the enzymatic nucleic acid component catalyzes a chemical reaction resulting in ligation of a predetermined RNA molecule to another predetermined RNA molecule.

26. A method comprising:
a. contacting the nucleic acid sensor molecule of claim 1 with a system comprising at least one ssRNA having a single nucleotide polymorphism (SNP) under conditions suitable for the enzymatic nucleic acid component of the nucleic acid sensor molecule to catalyze a chemical reaction resulting in a detectable response; and
b. assaying for said chemical reaction.

27. A method comprising:
a. contacting the nucleic acid sensor molecule of claim 2 with a system comprising at least one ssDNA having a single nucleotide polymorphism (SNP) under conditions suitable for the enzymatic nucleic acid component of the nucleic acid sensor molecule to catalyze a chemical reaction resulting in a detectable response; and
b. assaying for said chemical reaction resulting in a detectable response.

28. The nucleic acid sensor molecule of claim 20 or claim 21, wherein said chemical reaction is cleavage of a phosphodiester internucleotide linkage.

29. The nucleic acid sensor molecule of claim 20 or claim 21, wherein said chemical reaction is ligation of a predetermined nucleic acid molecule to the nucleic acid sensor molecule.

30. The nucleic acid sensor molecule of claim 20 or claim 21, wherein said chemical reaction is ligation of a predetermined nucleic acid molecule to another predetermined nucleic acid molecule.
